(19) United States
(12) Patent Application Publication
(10) Pub. No.: US 2009/0233907 A1
(43) Pub. Date: Sep. 17, 2009

(54) Pyridopyrimidine Derivatives and Their Use as PDE4 Inhibitors

(75) Inventors: Rupert Austin, Leicestershire (GB); Roger Victor Bonnert, Leicestershire (GB); Simon Fraser Hunt, Leicestershire (GB); Grigorios Nikitidis, Lund (SE); Hitesh Jayantilal Sanganee, Leicestershire (GB); Peter Olof Sjo, Lund (SE); Dan Warner, Leicestershire (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 12/293,480

(22) PCT Filed: Mar. 20, 2007
(86) PCT No.: PCT/SE2007/000269

§ 371 Date: Sep. 18, 2008

Related U.S. Application Data
(60) Provisional application No. 60/784,855, filed on Mar. 22, 2006.
(60) Provisional application No. 60/884,456, filed on Jan. 11, 2007.

(51) Int. Cl. A61K 31/551; A61K 31/535; A61K 31/5377; A61K 31/519; C07D 243/00; C07D 413/14; C07D 471/04; C07D 487/00

(52) U.S. Cl. 514/217.04; 514/229.2; 514/235.8; 514/259.31; 514/264.1; 540/553; 544/65; 544/120; 544/279; 544/281

(57) ABSTRACT

The present invention provides a compound of a formula (I): wherein the variables are defined herein; to a process for preparing such a compound; and to the use of such a compound in the treatment of a PDE 4 mediated disease state.

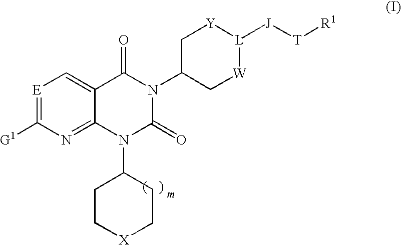

The present invention concerns pyridopyrimidine derivatives having pharmaceutical activity, to processes for preparing such derivatives, to pharmaceutical compositions comprising such derivatives and to the use of such derivatives as active therapeutic agents.

Pharmaceutically active pyridopyrimidine derivatives are disclosed in EP-A-0260817, WO 98/02162, WO 93/19068 and WO 0045800.

Phosphodiesterases (PDEs) work by converting cAMP or cGMP to AMP and GMP, or the inactive nucleotide forms incapable of activating downstream signalling pathways. The inhibition of PDEs leads to the accumulation of cAMP or cGMP, and subsequent activation of downstream pathways. PDEs comprise a large family of second messengers with 11 families and over 50 isoforms. In addition splice variants have been described for each isoform. The PDEs can be cAMP-specific (PDE4, 7, 8, 10), cGMP specific (PDE5, 6, 9) or have dual specificity (PDE1, 2, 3, 11).

cAMP is generated from ATP at the inner leaflet of the plasma membrane through the action of GPCR-regulated adenylate cyclase. Once cAMP is generated, the only way to terminate the signal is through phosphodiesterase action, degrading cAMP into 5′-AMP. Increased concentrations of cAMP are translated into cellular responses mainly by activation of cAMP-dependent protein kinase (PKA). The specific activity of PKA is in part regulated by the sub-cellular localization of PKA, which limits the phosphorylation of PKA to substrates in its near vicinity. The downstream events caused by activation of PKA appear poorly elucidated and involve many components in the initiation of signalling cascades. PDE4s have been shown to have abundant roles in regulating cell desensitisation, adaptation, signal cross-talk, cAMP compartmentalization and feedback loops, and are major regulators of cAMP homeostasis.

The physiological role implicated for elevated cAMP levels include: 1) broad suppression the activity of many immunocompetent cells; 2) induction of airway smooth muscle relaxation; 3) suppression of smooth muscle mitogenesis; and, 4) has beneficial modulatory effects on the activity of pulmonary nerves.

PDE4 has been found to be the predominant cAMP metabolising isozyme family in immune and inflammatory cells and, along with the PDE3 family, a major contributor to cAMP metabolism in airway smooth muscle.

Over the last two decades significant attention has been devoted into the development of PDE4 selective inhibitors for the treatment of inflammatory and immune disorders including asthma, rhinitis, bronchitis, COPD, arthritis and psoriasis. A number of compounds (for example rolipram, tibenelast and denbufylline) have been reported to have impressive effects in animal models of inflammation, especially pulmonary inflammation.

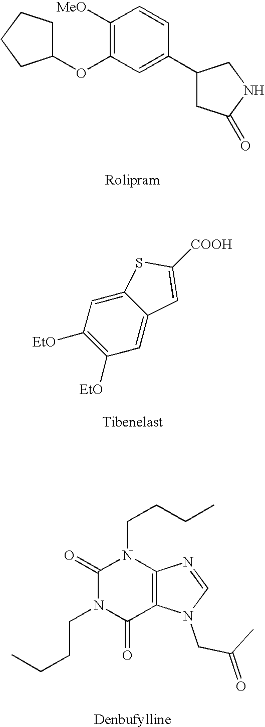

Unfortunately the clinical utility of these inhibitors has been limited by PDE4 related side-effects, including nausea, vomiting and gastric acid secretion. Recently a second generation of PDE4 inhibitors (for example cilomilast, roflumilast and AWD 12-281) has been described having significantly reduced risk of emetic side effects in animal models of emesis, thus providing the potential for an increased therapeutic ratio.

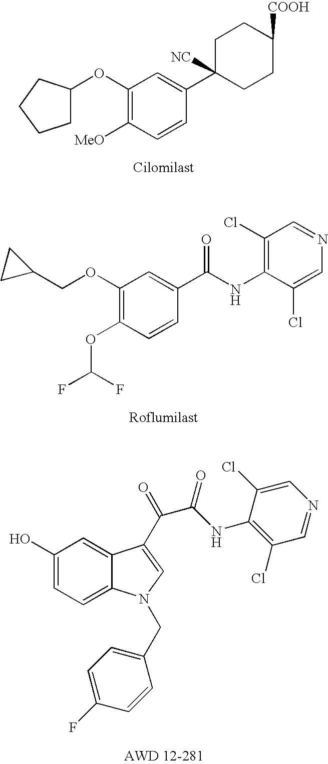

The present invention discloses novel pyridopyrimidine derivatives that are inhibitors of human PDE4 and are thereby useful in therapy.

The present invention provides a compound of formula (I):

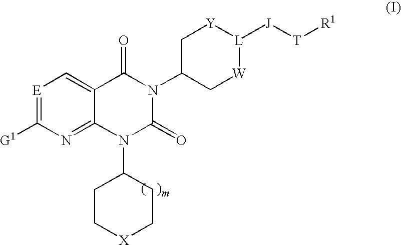

wherein:

X is S, S(O) or S(O)₂;

E is N or CE¹;

T is C(O) or S(O)₂;

W is (CH₂)ₙ;

Y is (CH₂)ₚ;

n and p are, independently 0 or 1;

m is 0 or 1;

L is CH or N;

when L is CH then J is NH; and when L is N then J is absent and T is bonded directly to L;

R¹ is C₁₋₁₀ alkyl (optionally substituted by halogen, hydroxyl, cyano, C₁₋₆ alkoxy, C₁₋₆ alkylthio, C₁₋₆ alkoxy(C₁₋₆ alkoxy), aryl, heteroaryl, aryloxy, heteroaryloxy, arylthio, heteroarylthio, aryl(C₁₋₄ alkoxy), aryl(C₁₋₄ alkylthio), C₃₋₇ cycloalkyl (optionally substituted by hydroxy, C₁₋₄ alkyl, C₁₋₄ alkoxy, or phenyl), CO₂H, CO₂(C₁₋₁₆ alkyl), NHC(O)R³, tetrahydropyranyl, C(O)NR²⁴R²⁵, S(O)₂R²⁶ or NHS(O)₂R²⁷), C₁₋₆ alkoxy, C₂₋₄ alkenyl (optionally substituted by phenyl), C₃₋₆ cycloalkyl (optionally substituted by hydroxyl, halogen, C₁₋₆ alkyl, C₁₋₆ alkoxy or phenyl), heterocyclyl (optionally substituted by oxo, hydroxy, C₁₋₆ alkyl, S(O)₂(C₁₋₄ alkyl), C(O)(C₁₋₄ alkyl), aryl, heteroaryl, aryl(C₁₋₄ alkyl), C(O)heteroaryl or heterocyclyl), aryl or heteroaryl;

R³ is C₁₋₆ alkyl or phenyl;

the foregoing phenyl, aryl and heteroaryl moieties of R¹ and R³ are, independently, optionally substituted by: halogen, cyano, nitro, hydroxy, S(O)_{q}R⁴, OC(O)NR⁵R⁶, NR⁷R⁸, NR⁹C(O)R¹⁰, NR¹¹C(O)NR¹²R¹³S(O)₂NR¹⁴R¹⁵, NR¹⁶S(O)₂R¹⁷, C(O)NR¹⁸R¹⁹, C(O)R²⁰, CO₂R²¹, NR²²CO₂R²³, C₁₋₁₀ alkyl, C₁₋₆ hydroxyalkyl, C₁₋₆ haloalkyl, C₁₋₆ alkoxy(C₁₋₆)alkyl, C₁₋₆ alkyl(S(O)₂(C₁₋₄ alkyl)), di(C₁₋₆)alkylamino(C₁₋₆)alkyl, C₁₋₆ alkoxy, C₁₋₆ haloalkoxy, C₁₋₆ alkoxy(C₁₋₆)alkoxy, C₁₋₆ alkylthio, C₂₋₆ alkenyl, C₂₋₆ alkynyl (optionally substituted by phenyl), C₃₋₁₀ cycloalkyl (itself optionally substituted by C₁₋₄ alkyl or oxo), methylenedioxy, OCH₂CH₂O, difluoromethylenedioxy, heterocyclyl (optionally substituted by hydroxy, C₁₋₄ alkyl, phenyl or heteroaryl), phenyl, phenyl(C₁₋₄)alkyl, phenoxy, phenylthio, phenyl(C₁₋₄)alkoxy, heteroaryl, heteroaryl(C₁₋₄)alkyl, heteroaryloxy or heteroaryl(C₁₋₄)alkoxy; wherein any of the immediately foregoing phenyl and heteroaryl moieties are optionally substituted with halogen, hydroxy, nitro, S(O)ᵣ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃;

E¹ and G¹ are, independently, hydrogen, halogen, cyano, hydroxy, C₁₋₄ alkyl, C₁₋₄ alkoxy, CF₃ or OCF₃;

q and r are, independently, 0, 1 or 2;

R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶ and R²⁷ are, independently, C₁₋₁₀ alkyl {optionally substituted by halogen, hydroxy or C₁₋₆ alkoxy, or phenyl or heteroaryl both optionally substituted as recited below}, CH₂(C₂₋₆ alkenyl), phenyl {itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃} or heteroaryl {itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₄ alkyl), NHS(O)₂(C₁₋₄ alkyl), C(O)(C₁₋₄ alkyl), CF₃ or OCF₃};

R⁵R⁶, R⁷, R⁸, R⁹, R¹⁰, R¹¹, R¹², R¹³, R¹⁴, R¹⁵, R¹⁶, R¹⁸, R¹⁹, R²⁰, R²¹, R²², R²³, R²⁴ and R²⁵ can also be hydrogen;

or a N-oxide thereof; or a pharmaceutically acceptable salt thereof.

Certain compounds of the present invention can exist in different isomeric forms (such as enantiomers, diastereomers, geometric isomers or tautomers). The present invention covers all such isomers and mixtures thereof in all proportions.

Suitable salts include acid addition salts such as a hydrochloride, dihydrochloride, hydrobromide, phosphate, sulfate, acetate, diacetate, fumarate, maleate, malonate, succinate, tartrate, citrate, oxalate, methanesulfonate or p-toluenesulfonate. Further examples of acid addition salts are: bisulphate, benzenesulphonate, ethanesulphonate, malonate, xinofoate, ascorbate, oleate, nicotinate, saccharinate, adipate, formate, glycolate, L-lactate, D-lactate, aspartate, malate, L-tartrate, D-tartrate, stearate, 2-furoate, 3-furoate, napadisylate (naphthalene-1,5-disulfonate or naphthalene-1-(sulfonic acid)-5-sulfonate), edisylate (ethane-1,2-disulfonate or ethane-1-(sulfonic acid)-2-sulfonate), isethionate (2-hydroxyethylsulfonate), 2-mesitylenesulphonate and 2-naphthalenesulphonate.

The compounds of the invention may exist as solvates (such as hydrates) and the present invention covers all such solvates.

Halogen includes fluorine, chlorine, bromine and iodine. Halogen is, for example, fluorine or chlorine.

Alkyl moieties are straight or branched chain and are, for example, methyl, ethyl, n-propyl, iso-propyl or tert-butyl. Haloalkyl is, for example C₂F₅, CF₃ or CHF₂. Alkoxy is, for example, methoxy or ethoxy; and haloalkoxy is, for example OCF₃ or OCHF₂.

Alkenyl is, for example, vinyl or prop-2-enyl. Alkynyl is, for example, propargyl.

Cycloalkyl is, for example, cyclopropyl, cyclobutyl, cyclopentyl or cyclohexyl; or cycloalkyl can be fused to a benzene ring to form, for example, indanyl. Cycloalkyloxy is, for example, cyclopropyloxy, cyclopentyloxy or cyclohexyloxy. Cycloalkylalkoxy is, for example, (cyclopropyl)methoxy or 2-(cyclopropyl)ethoxy.

Heterocyclyl is a non-aromatic 5- or 6-membered ring optionally fused to one or more other non-aromatic rings and optionally fused to a benzene or heteroaromatic (such as a heteroaryl ring below) ring, comprising at least one heteroatom selected from the group comprising nitrogen, oxygen and sulphur. Heterocyclyl is, for example, azetidinyl, pyrrolidinyl, piperidinyl, piperazinyl, isoindolyl, morpholinyl, 3,8-diazabicyclo[3.2.1]octyl, 8-azabicyclo[2.2.2]octyl, 2-oxa-6-azabicyclo[5.4.0]undeca-7,9,11-trienyl, 7-oxa-10-azabicyclo[4.4.0]deca-1,3,5-trienyl, 6-thia-1,4-diazabicyclo[3.3.0]octa-4,7-dienyl, tetrahydropyranyl, azabicyclo[3.2.1]octyl, 1,2,3,4-tetrahydroquinolinyl, 1,4-diazepinyl, quinuclidinyl, 9-oxa-2,8-diazaspiro[4.4]non-7-enyl, 1,2-dihydroquinazolinyl, 2,4,10-triazabicyclo[4.4.0]deca-1,3,5,8-tetraenyl, 2-oxa-5-aza-bicyclo[4.4.0]deca-7,9,11-trienyl, adamantyl, tetrahydrofuranyl or thiazinanyl (for example as an S-dioxide).

Hydroxyalkyl is, for example, CH₂OH; C₁₋₆ alkoxy(C₁₋₆)alkyl is, for example CH₃OCH₂; and, C₁₋₆ alkoxy(C₁₋₆)alkoxy is, for example, CH₃OCH₂O. Dialkylaminoalkyl is, for example (CH₃)₂NCH₂ or (CH₃)(CH₃CH₂)NCH₂.

Aryl is, for example, phenyl or naphthyl. Aryl is, for example phenyl.

Heteroaryl is, for example, an aromatic 5- or 6-membered ring, optionally fused to one or more other rings, comprising at least one heteroatom selected from the group comprising nitrogen, oxygen and sulphur; or an N-oxide thereof, or an S-oxide or S-dioxide thereof. Heteroaryl is, for example, furyl, thienyl (also known as thiophenyl), pyrrolyl, thiazolyl, isothiazolyl, pyrazolyl, oxazolyl, isoxazolyl, imidazolyl, [1,2,3]-thiadiazolyl, [1,2,4]-triazolyl, [1,2,3]-triazolyl, pyridinyl, pyrimidinyl, pyrazinyl, pyridazinyl, indolyl, benzo[b]furyl (also known as benzofuryl), benz[b]thienyl (also known as benzothienyl or benzothiophenyl), indazolyl, benzimidazolyl, 1,2,3-benztriazolyl, benzoxazolyl, 1,3-benzthiazolyl, 1,2,3-benzothiadiazolyl, an imidazopyridinyl (such as imidazo[1,2a]pyridinyl), thieno[3,2-b]pyridin-6-yl, 1,2,3-benzoxadiazolyl (also known as benzo[1,2,3]thiadiazolyl), 2,1,3-benzothiadiazolyl, benzofurazan (also known as 2,1,3-benzoxadiazolyl), quinoxalinyl, a pyrazolopyridine (for example 1H-pyrazolo[3,4-b]pyridinyl or pyrazolo[1,5-a]pyridinyl), an imidazopyridine (for example imidazo[1,2-a]pyridinyl), a dihydropyrido[2,3-d]pyrimidine (for example 1,4-dihydropyrido[2,3-d]pyrimidinyl), quinolinyl, isoquinolinyl, a naphthyridinyl (for example [1,6]naphthyridinyl, [1,7]naphthyridinyl or [1,8]naphthyridinyl), 1,2,3-thiadiazolyl, 1H-pyrrolo[2,3-b]pyridinyl, thieno[2,3-b]pyridinyl, thieno[2,3-b]pyrazinyl, [1,2,4]triazolo[1,5-a]pyrimidinyl or 6,7-dihydro-5H-[1,3]thiazolo[3,2-a]pyrimidinyl; or an N-oxide thereof, or an S-oxide or S-dioxide thereof.

In one particular aspect the present invention provides a compound of formula (I) wherein: X is S, S(O) or S(O)₂; E is N or CE¹; T is C(O) or S(O)₂; W is (CH₂)ₙ; Y is (CH₂)ₚ; n and p are, independently 0 or 1; m is 0 or 1; L is CH or N; when L is CH then J is NH; and when L is N then J is absent and T is bonded directly to L; R¹ is C₁₋₁₀ alkyl (optionally substituted by halogen, hydroxyl, cyano, C₁₋₆ alkoxy, C₁₋₆ alkylthio, C₁₋₆ alkoxy(C₁₋₆ alkoxy), aryl, heteroaryl, aryloxy, heteroaryloxy, aryl(C₁₋₄ alkoxy), aryl(C₁₋₄ alkylthio), C₃₋₇ cycloalkyl (optionally substituted by C₁₋₄ alkyl), CO₂H, CO₂(C₁₋₆ alkyl), NHC(O)R³, tetrahydropyranyl, C(O)NR²⁴R²⁵, S(O)₂R²⁶ or NHS(O)₂R²⁷), C₁₋₆ alkoxy, C₂₋₄ alkenyl (optionally substituted by phenyl), C₃₋₆ cycloalkyl (optionally substituted by hydroxyl, halogen, C₁₋₆ alkyl, C₁₋₆ alkoxy or phenyl), heterocyclyl (optionally substituted by oxo, hydroxy, C₁₋₆ alkyl, S(O)₂(C₁₋₄ alkyl), C(O)(C₁₋₄ alkyl), aryl, heteroaryl, aryl(C₁₋₄ alkyl), C(O)heteroaryl or heterocyclyl), aryl or heteroaryl; R³ is C₁₋₆ alkyl or phenyl; the foregoing phenyl, aryl and heteroaryl moieties of R¹ and R³ are, independently, optionally substituted by: halogen, cyano, nitro, hydroxy, S(O)_{q}R⁴, OC(O)NR⁵R⁶, NR⁷R⁸, NR⁹C(O)R¹⁰, NR¹¹C(O)NR¹²R¹³, S(O)₂NR¹⁴R¹⁵, NR¹⁶S(O)₂R¹⁷, C(O)NR¹⁸R¹⁹, C(O)R²⁰, CO₂R²¹, NR²²CO₂R²³, C₁₋₁₀ alkyl, C₁₋₆ hydroxyalkyl, C₁₋₆ haloalkyl, C₁₋₆ alkoxy(C₁₋₆)alkyl, C₁₋₆ alkyl(S(O)₂(C₁₋₄ alkyl)), di(C₁₋₆)alkylamino(C₁₋₆)alkyl, C₁₋₆ alkoxy, C₁₋₆ haloalkoxy, C₁₋₆ alkoxy(C₁₋₆)alkoxy, C₁₋₆ alkylthio, C₂₋₆ alkenyl, C₂₋₆ alkynyl (optionally substituted by phenyl), C₃₋₁₀ cycloalkyl (itself optionally substituted by C₁₋₄ alkyl or oxo), methylenedioxy, OCH₂CH₂O, difluoromethylenedioxy, heterocyclyl (optionally substituted by hydroxy, C₁₋₄ alkyl, phenyl or heteroaryl), phenyl, phenyl(C₁₋₄)alkyl, phenoxy, phenylthio, phenyl(C₁₋₄)alkoxy, heteroaryl, heteroaryl(C₁₋₄)alkyl, heteroaryloxy or heteroaryl(C₁₋₄)alkoxy; wherein any of the immediately foregoing phenyl and heteroaryl moieties are optionally substituted with halogen, hydroxy, nitro, S(O)ᵣ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; E¹ and G¹ are, independently, hydrogen, halogen, cyano, hydroxy, C₁₋₄ alkyl, C₁₋₄ alkoxy, CF₃ or OCF₃; q and r are, independently, 0, 1 or 2; R⁴, R⁵, R⁶, R⁷, R⁸, R⁹, R¹⁰R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶ and R²⁷ are, independently, C₁₋₁₀ alkyl {optionally substituted by halogen, hydroxy or C₁₋₆ alkoxy, or phenyl or heteroaryl both optionally substituted as recited below}, CH₂(C₂₋₆ alkenyl), phenyl {itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃} or heteroaryl {itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}; R⁵, R⁶, R⁷, R⁸, R⁹, R¹⁰, R¹¹, R¹², R¹³, R¹⁴, R¹⁵, R¹⁶, R¹⁸, R¹⁹, R²⁰, R²¹, R²², R²³, R²⁴ and R²⁵ can also be hydrogen; or a N-oxide thereof; or a pharmaceutically acceptable salt thereof.

In a further aspect the present invention provides a compound of formula (I) wherein: X is S; E is N or CE¹; T is C(O) or S(O)₂; W is (CH₂)ₙ; Y is (CH₂)ₚ; n and p are, independently 0 or 1; m is 0 or 1; L is CH or N; when L is CH then J is NH; and when L is N then J is absent and T is bonded directly to L; R¹ is C₁₋₆ alkyl (optionally substituted by hydroxyl, C₁₋₆ alkoxy, aryl, heteroaryl, C₃₋₇ cycloalkyl, CO₂H, CO₂(C₁₋₆ alkyl) or NHC(O)R³), C₁₋₆ alkoxy, C₃₋₆ cycloalkyl (optionally substituted by hydroxyl or C₁₋₆ alkyl), heterocyclyl (optionally substituted by C₁₋₆ alkyl), aryl or heteroaryl; R³ is C₁₋₆ alkyl or phenyl; the foregoing phenyl, aryl and heteroaryl moieties of R¹ and R³ are, independently, optionally substituted by: halogen, cyano, nitro, hydroxy, S(O)_{q}R⁴, OC(O)NR⁵R⁶, NR⁷R⁸, NR⁹C(O)R¹⁰, NR¹¹C(O)NR¹²R¹³, S(O)₂NR¹⁴R¹⁵, NR¹⁶S(O)₂R¹⁷, C(O)NR¹⁸R¹⁹, C(O)R²⁰, CO₂R²¹, NR²²CO₂R²³, C₁₋₆ alkyl, C₁₋₆ hydroxyalkyl, C₁₋₆ haloalkyl, C₁₋₆ alkoxy(C₁₋₆)alkyl, di(C₁₋₆)alkylamino(C₁₋₆)alkyl, C₁₋₆ alkoxy, C₁₋₆ haloalkoxy, C₁₋₆ alkoxy(C₁₋₆)alkoxy, C₁₋₆ alkylthio, C₂₋₆ alkenyl, C₂₋₆ alkynyl, C₃₋₁₀ cycloalkyl (itself optionally substituted by C₁₋₄ alkyl or oxo), methylenedioxy, difluoromethylenedioxy, phenyl, phenyl(C₁₋₄)alkyl, phenoxy, phenylthio, phenyl(C₁₋₄)alkoxy, heteroaryl, heteroaryl(C₁₋₄)alkyl, heteroaryloxy or heteroaryl(C₁₋₄)alkoxy; wherein any of the immediately foregoing phenyl and heteroaryl moieties are optionally substituted with halogen, hydroxy, nitro, S(O)ᵣ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; E¹ and G¹ are, independently, hydrogen, halogen, cyano, hydroxy, C₁₋₄ alkyl, C₁₋₄ alkoxy, CF₃ or OCF₃; q and r are, independently, 0, 1 or 2; R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ are, independently, C₁₋₆ alkyl {optionally substituted by halogen, hydroxy or C₁₋₆ alkoxy}, CH₂(C₂₋₆ alkenyl), phenyl {itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃} or heteroaryl {itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}; R⁵, R⁶, R⁷, R⁸, R⁹, R¹⁰, R¹¹, R¹², R¹³, R¹⁴, R¹⁵, R¹⁶, R¹⁸, R¹⁹, R²⁰, R²¹, R²² and R²³ can also be hydrogen; or a N-oxide thereof; or a pharmaceutically acceptable salt thereof.

In another aspect the present invention provides a compound of formula (I) wherein E is CE¹. In a further aspect the present invention provides a compound of formula (I) wherein E¹ is hydrogen or halogen (for example fluoro).

In yet another aspect the present invention provides a compound of formula (I) wherein n and p are both 1.

In a further aspect the present invention provides a compound of formula (I) wherein L is CH.

In another aspect the present invention provides a compound of formula (I) wherein L is CH, J is NH and T is C(O).

In a still further aspect the present invention provides a compound of formula (I) wherein Y and W are both CH₂, L is CH, J is NH and T is C(O).

In another aspect the present invention provides a compound of formula (I) wherein E¹ and G¹ are, independently, hydrogen or halogen (for example fluoro).

In a further aspect the present invention provides a compound of formula (I) wherein G¹ is hydrogen.

In yet another aspect the present invention provides a compound of formula (I) wherein n is 0.

In a further aspect the present invention provides a compound of formula (I) wherein n is 1.

In a still further aspect the present invention provides a compound of formula (I) wherein m is 1.

In another aspect the present invention provides a compound of formula (I) wherein X is S.

In yet another aspect the present invention provides a compound of formula (I) wherein R¹ is C₁₋₆ alkyl (optionally substituted by halogen, hydroxyl, C₁₋₆ alkoxy, C₃₋₆ cycloalkyl (optionally substituted by hydroxyl, C₁₋₄ alkyl, C₁₋₄ alkoxy or phenyl), aryl or heteroaryl), C₃₋₇ cycloalkyl (optionally substituted by hydroxyl, halogen, C₁₋₆ alkyl, C₁₋₆ alkoxy or phenyl), heterocyclyl (optionally substituted by oxo, hydroxy, C₁₋₆ alkyl, S(O)₂(C₁₋₄ alkyl), C(O)(C₁₋₄ alkyl), aryl, heteroaryl, aryl(C₁₋₄ alkyl), C(O)heteroaryl or heterocyclyl), aryl or heteroaryl; the foregoing phenyl, aryl and heteroaryl moieties of R¹ are, independently, optionally substituted by: halogen, cyano, hydroxy, S(O)₂R⁴, NR⁷R⁸, NR¹¹C(O)NR¹²R¹³, S(O)₂NR¹⁴R¹⁵, C(O)NR¹⁸R¹⁹, C₁₋₁₀ alkyl, C₁₋₆ hydroxyalkyl, C₁₋₆ haloalkyl, C₁₋₆ alkoxy(C₁₋₆)alkyl, C₁₋₆ alkyl(S(O)₂(C₁₋₄ alkyl)), di(C₁₋₆)alkylamino(C₁₋₆)alkyl, C₁₋₆ alkoxy, C₁₋₆ haloalkoxy, C₁₋₆ alkoxy(C₁₋₆)alkoxy, C₃₋₁₀ cycloalkyl (itself optionally substituted by C₁₋₄ alkyl or oxo) or heterocyclyl (optionally substituted by hydroxy, C₁₋₄ alkyl, phenyl or heteroaryl); R⁴ is C₁₋₄ alkyl; and R⁷, R⁸, R¹¹, R², R¹³, R¹⁴, R¹⁵, R¹⁸ and R¹⁹ are, independently, hydrogen or C₁₋₄ alkyl.

In a further aspect the present invention provides a compound of formula (I) wherein R¹ is C₁₋₆ alkyl (optionally substituted by hydroxy, aryl, C₃₋₇ cycloalkyl, CO₂(C₁₋₆ alkyl) or NHC(O)R³), aryl or heteroaryl (for example pyridyl, benzimidazolyl, furyl or 1,4-dihydropyrido[2,3-d]pyrimidinyl); and R³ is phenyl.

Aryl is, for example, phenyl.

In a still further aspect the present invention provides a compound of formula (I) wherein the foregoing phenyl, aryl and heteroaryl moieties of R¹ and R³ are, independently, optionally substituted by: halogen, hydroxy, CO₂(C₁₋₆ alkyl), C₁₋₆ alkyl, C₁₋₆ hydroxyalkyl or C₁₋₆ alkoxy.

In another aspect the present invention provides a compound of formula (I) wherein R¹ is heterocyclyl (optionally substituted by C₁₋₆ alkyl), for example R¹ is pyrrolyl, piperidinyl or morpholinyl, each optionally substituted by C₁₋₆ alkyl (such as methyl or ethyl).

In yet another aspect the present invention provides a compound of formula (I) wherein R¹ is C₁₋₄ alkyl {substituted by heteroaryl (such as an imidazo[1,2-a]pyridinyl)} or heteroaryl (such as an imidazo[1,2-a]pyridinyl); heteroaryl being optionally substituted by halogen, C₁₋₄ alkyl, CF₃, C₁₋₄ alkoxy, OCF₃ or heterocyclyl (such as pyrrolidinyl); heterocyclyl being optionally substituted by C₁₋₄ alkyl.

In another aspect the present invention provides a compound of formula (I) wherein R¹ is C₁₋₄ alkyl (for example methyl or ethyl) optionally substituted by OH, C₁₋₄ alkoxy, phenyl (itself optionally substituted by OH, halogen, C₁₋₄ alkoxy or C₁₋₄ alkyl) or C₃₋₇ cycloalkyl (itself optionally substituted by OH, phenyl, C₁₋₄ alkoxy or C₁₋₄ alkyl).

In yet another aspect the present invention provides a compound of formula (I) wherein R¹ is C₁₋₄ alkyl (eg methyl or ethyl) substituted by either OH or C₁₋₄ alkoxy (for example by OH), and further substituted by phenyl (itself optionally substituted by OH, halogen, C₁₋₄ alkoxy or C₁₋₄ alkyl).

In a further aspect the present invention provides a compound of formula (I) wherein R¹ is methyl substituted by OH, and further substituted by phenyl (itself optionally substituted by OH, halogen, C₁₋₄ alkoxy or C₁₋₄ alkyl) {for example further substituted by phenyl (itself substituted (for example by 1) by OH)}.

In a still further aspect the present invention provides a compound of formula (I) wherein R¹ is C₁₋₄ alkyl (eg methyl or ethyl) substituted by phenyl (itself optionally substituted by OH, halogen, C₁₋₄ alkoxy or C₁₋₄ alkyl) or C₃₋₇ cycloalkyl (itself optionally substituted by OH, phenyl, C₁₋₄ alkoxy or C₁₋₄ alkyl).

In another aspect the present invention provides a compound of formula (I) wherein R¹ is a mono-cyclic heterocyclyl optionally substituted (for example at the point of attachment to the remainder of formula (I), such as in Example 109a) by phenyl (itself optionally substituted by CF₃, OCF₃, CN, OH, C₁₋₄ alkyl, C₁₋₄ alkoxy or halogen); and the heterocyclyl contains one S, O or N atom in the ring, and is 3- to 8-membered.

In yet another aspect the present invention provides a compound of formula (I) wherein R¹ is C₃₋₇ cycloalkyl optionally substituted (for example at the point of attachment to the remainder of formula (I), such as in Example 122) by phenyl (itself optionally substituted by CF₃, OCF₃, OH, CN, C₁₋₄ alkyl, C₁₋₄ alkoxy or halogen).

In a further aspect the present invention provides a compound of formula (I) wherein R¹ is aryl (for example phenyl) optionally substituted by halogen, C₁₋₄ alkyl, C₁₋₄ alkoxy or OH).

In a still further aspect the present invention provides a compound of formula (I) wherein R¹ is heteroaryl (for example pyrimidinyl, pyridinyl, imidazo[1,2-a]pyridinyl, pyrazolo[1,5-a]pyridinyl or indazolyl) optionally substituted by halogen (such as fluoro), C₁₋₄ alkyl, C₁₋₄ alkoxy, CF₃, OCF₃, OH, CN, C₁₋₄ alkylthio, phenylthio or heterocyclyl (optionally substituted by OH, or C₁₋₄ alkyl).

In another aspect the present invention provides a compound of formula (I) wherein R¹ is pyrimidinyl, pyridinyl or imidazo[1,2-a]pyridinyl, all optionally substituted by halogen (such as fluoro), C₁₋₄ alkyl or C₁₋₄ alkoxy.

In yet another aspect the present invention provides a compound of formula (I) wherein R¹ is C₁₋₄ alkyl substituted by: phenoxy, heteroaryloxy or heteroarylthio, each optionally substituted by halogen, CF₃, OCF₃, OH, CN, C₁₋₄ alkyl, C₁₋₄ alkoxy or tetrazolyl.

In a further aspect the present invention provides a compound of formula (I) wherein, when L is CH, the substituents are cis-disposed on the ring marked * below.

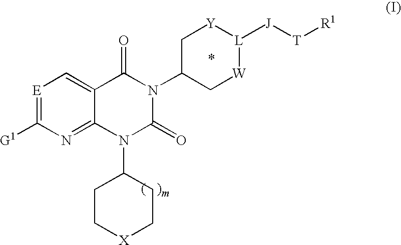

Compounds of the invention are described in the Examples. Each of the compounds of the Examples, or a pharmaceutically acceptable salt thereof, is a further aspect of the present invention. Thus the present invention provides a compound:
N-{cis-4-[6-Fluoro-2,4-dioxo-1-(tetrahydro-2H-thiopyran-4-yl)-1,4-dihydropyrido[2,3-d]pyrimidin-3(2H)-yl]cyclohexyl}cyclopropanecarboxamide;
N-{cis-4-[6-Fluoro-2,4-dioxo-1-(tetrahydro-2H-thiopyran-4-yl)-1,4-dihydropyrido[2,3-d]pyrimidin-3(2H)-yl]cyclohexyl}imidazo[1,2-a]pyridine-2-carboxamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}quinoline-8-carboxamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}pyrazolo[1,5-a]pyridine-2-carboxamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-1H-indazole-3-carboxamide;
N-[2-({cis-4-[6-fluoro-2,4-dioxo-1-(tetrahydro-2H-thiopyran-4-yl)-1,4-dihydropyrido[2,3-d]pyrimidin-3(2H)-yl]cyclohexyl}amino)-2-oxoethyl]-2-hydroxybenzamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-3-hydroxy-4-methoxybenzamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-2-(3-hydroxyphenyl)acetamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-2-hydroxy-4-methylbenzamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-3-hydroxy-4-methylbenzamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-4-hydroxy-3-methoxybenzamide;
(2R)-2-cyclohexyl-N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-2-hydroxyacetamide;
methyl 5-({cis-4-[6-fluoro-2,4-dioxo-1-(tetrahydro-2H-thiopyran-4-yl)-1,4-dihydropyrido[2,3-d]pyrimidin-3(2H)-yl]cyclohexyl}amino)-5-oxopentanoate;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-3-hydroxybenzamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-2-(4-hydroxyphenyl)acetamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-4-hydroxybenzamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-2-hydroxybenzamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}cyclohexanecarboxamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-3-(3-hydroxyphenyl)propanamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-3-hydroxypyridine-2-carboxamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-3-methylbutanamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-2-hydroxy-2-phenylacetamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}benzamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-2-furamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}imidazo[1,2-a]pyridine-8-carboxamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-2-hydroxy-5-(hydroxymethyl)benzamide;
N-{cis-4-[6-fluoro-2,4-dioxo-1-(tetrahydro-3-thienyl)-1,4-dihydropyrido[2,3-d]pyrimidin-3(2H)-yl]cyclohexyl}cyclopropanecarboxamide;
N-{cis-4-[6-fluoro-2,4-dioxo-1-(tetrahydro-3-thienyl)-1,4-dihydropyrido[2,3-d]pyrimidin-3(2H)-yl]cyclohexyl}imidazo[1,2-a]pyridine-2-carboxamide;
N-{cis-4-[6-fluoro-2,4-dioxo-1-(tetrahydro-3-thienyl)-1,4-dihydropyrido[2,3-d]pyrimidin-3(2H)-yl]cyclohexyl}-2-hydroxy-5-(hydroxymethyl)benzamide;
N-{cis-4-[6-fluoro-2,4-dioxo-1-(tetrahydro-3-thienyl)-1,4-dihydropyrido[2,3-d]pyrimidin-3(2H)-yl]cyclohexyl}-2-hydroxybenzamide;
N-{cis-4-[6-fluoro-2,4-dioxo-1-(tetrahydro-3-thienyl)-1,4-dihydropyrido[2,3-d]pyrimidin-3(2H)-yl]cyclohexyl}-2-hydroxy-4-methylbenzamide;
N-{cis-4-[2,4-dioxo-1-(tetrahydro-2H-thiopyran-4-yl)-1,4-dihydropyrido[2,3-d]pyrimidin-3(2H)-yl]cyclohexyl}imidazo[1,2-a]pyridine-2-carboxamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-5,6,7,8-tetrahydroimidazo[1,2-a]pyridine-2-carboxamide;
N-{cis-4-[6-methyl-2,4-dioxo-1-(tetrahydro-2H-thiopyran-4-yl)-1,4-dihydropyrido[2,3-d]pyrimidin-3(2H)-yl]cyclohexyl}imidazo[1,2-a]pyridine-2-carboxamide;
N-{cis-4-[6-Fluoro-1-[(1r)-1-oxidotetrahydro-2H-thiopyran-4-yl]-2,4-dioxo-1,4-dihydropyrido[2,3-d]pyrimidin-3(2H)-yl]cyclohexyl}cyclopropanecarboxamide;
N-{cis-4-[6-fluoro-1-(1-oxidotetrahydro-2H-thiopyran-4-yl)-2,4-dioxo-1,4-dihydropyrido[2,3-d]pyrimidin-3(2H)-yl]cyclohexyl}imidazo[1,2-a]pyridine-2-carboxamide;
N-{cis-4-[1-(1,1-Dioxidotetrahydro-2H-thiopyran-4-yl)-6-fluoro-2,4-dioxo-1,4-dihydropyrido[2,3-d]pyrimidin-3(2H)-yl]cyclohexyl}cyclopropanecarboxamide;
N-{cis-4-[6-Fluoro-1-[(1s)-1-oxidotetrahydro-2H-thiopyran-4-yl]-2,4-dioxo-1,4-dihydropyrido[2,3-d]pyrimidin-3(2H)-yl]cyclohexyl}cyclopropanecarboxamide;
N-{cis-4-[1-(1,1-Dioxidotetrahydro-2H-thiopyran-4-yl)-6-fluoro-2,4-dioxo-1,4-dihydropyrido[2,3-d]pyrimidin-3(2H)-yl]cyclohexyl}-2-hydroxy-5-(hydroxymethyl)benzamide;
N-{cis-4-[1-(1,1-Dioxidotetrahydro-2H-thiopyran-4-yl)-6-fluoro-2,4-dioxo-1,4-dihydropyrido[2,3-d]pyrimidin-3(2H)-yl]cyclohexyl}imidazo[1,2-a]pyridine-2-carboxamide;
6-fluoro-N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}imidazo[1,2-a]pyridine-2-carboxamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-2-hydroxy-2-(4-hydroxyphenyl)acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-nitro-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1H-indole-2-carboxamide;
4-cyano-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1H-benzotriazole-5-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]benzothiazole-6-carboxamide;
N-[4-(3-fluoro-8,10-dioxo-7-tetrahydrothiopyran-4-yl-5,7,9-triazabicyclo[4.4.0]deca-1,3,5-trien-9-yl)cyclohexyl]-9-methyl-7-thia-1,5-diazabicyclo[4.3.0]nona-5,8-diene-8-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]thiadiazole-4-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]thiophene-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-methyl-imidazole-4-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-methyl-piperidine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-vinyl-benzamide;
4-ethynyl-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]benzothiazole-2-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-9-thia-2-azabicyclo[4.3.0]nona-1,3,5,7-tetraene-8-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-9-thia-2,5-diazabicyclo[4.3.0]nona-1,3,5,7-tetraene-8-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1,5,7,9-tetrazabicyclo[4.3.0]nona-2,4,6,8-tetraene-8-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-methyl-1,7-diazabicyclo[4.3.0]nona-2,4,6,8-tetraene-8-carboxamide;
N-[4-(3-fluoro-8,10-dioxo-7-tetrahydrothiopyran-4-yl-5,7,9-triazabicyclo[4.4.0]deca-1,3,5-trien-9-yl)cyclohexyl]-3-methyl-1,7-diazabicyclo[4.3.0]nona-2,4,6,8-tetraene-8-carboxamide;
3-chloro-N-[4-(3-fluoro-8,10-dioxo-7-tetrahydrothiopyran-4-yl-5,7,9-triazabicyclo[4.4.0]deca-1,3,5-trien-9-yl)cyclohexyl]-1,7-diazabicyclo[4.3.0]nona-2,4,6,8-tetraene-8-carboxamide;
5-chloro-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(trifluoromethyl)-1,7-diazabicyclo[4.3.0]nona-2,4,6,8-tetraene-8-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1,5,7-triazabicyclo[4.3.0]nona-2,4,6,8-tetraene-8-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-5-methyl-1,7-diazabicyclo[4.3.0]nona-2,4,6,8-tetraene-8-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-1,7-diazabicyclo[4.3.0]nona-2,4,6,8-tetraene-8-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-6-imidazol-1-yl-pyridine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-[5-(trifluoromethyl)-2-pyridyl]piperidine-4-carboxamide;
4-dimethylamino-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide;
3-dimethylamino-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide;
4-diethylamino-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide;
10-ethyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-7-oxo-3-pyrrolidin-1-yl-2,4,10-triazabicyclo[4.4.0]deca-1,3,5,8-tetraene-8-carboxamide;
(E)-3-(4-dimethylaminophenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]prop-2-enamide;
2-dimethylamino-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-oxo-2-(2-pyridylmethyl)isoquinoline-4-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-pyrimidin-2-yl-piperidine-4-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-methyl-imidazole-2-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-morpholino-benzamide;
1-[(4-chlorophenyl)methyl]-N-[4-(9-fluoro-2,4-dioxo-5-tetrahydrothiopyran-4-yl-3,5,7-triazabicyclo[4.4.0]deca-6,8,10-trien-3-yl)cyclohexyl]-5-methyl-imidazole-4-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-(pyridine-3-carbonyl)piperidine-4-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-6-morpholino-pyrazine-2-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(2-methylimidazol-1-yl)propanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-imidazol-1-yl-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-(3-pyridyl)piperidine-4-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-6-(1-piperidyl)pyridine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-5-methyl-2-oxa-5-azabicyclo[4.4.0]deca-7,9,11-triene-9-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-morpholino-pyridine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1,2-dimethyl-benzoimidazole-5-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-morpholino-pyrimidine-5-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-imidazol-1-yl-butanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-6-(2-phenylpyrrolidin-1-yl)pyridine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(2-phenylpyrrolidin-1-yl)pyridine-3-carboxamide;
2-(4-dimethylaminophenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-7-methyl-8-oxo-5-phenyl-1,7-naphthyridine-6-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-5-oxo-1-(6-quinolyl)pyrrolidine-3-carboxamide;
2-chloro-N-[4-(9-fluoro-2,4-dioxo-5-tetrahydrothiopyran-4-yl-3,5,7-triazabicyclo[4.4.0]deca-6,8,10-trien-3-yl)cyclohexyl]-4-morpholino-benzamide;
1-ethyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-7-methyl-4-oxo-1,8-naphthyridine-3-carboxamide;
(2S)—N-[4-(9-fluoro-2,4-dioxo-5-tetrahydrothiopyran-4-yl-3,5,7-triazabicyclo[4.4.0]deca-6,8,10-trien-3-yl)cyclohexyl]-1-methyl-pyrrolidine-2-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-methyl-piperidine-4-carboxamide;
3-(3,4-dihydro-2H-quinolin-1-yl)-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methyl-4-oxo-1,2-dihydroquinazoline-2-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]piperidine-3-carboxamide;
(3R)—N-[4-(9-fluoro-2,4-dioxo-5-tetrahydrothiopyran-4-yl-3,5,7-triazabicyclo[4.4.0]deca-6,8,10-trien-3-yl)cyclohexyl]piperidine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]piperidine-2-carboxamide;
(2S)—N-[4-(9-fluoro-2,4-dioxo-5-tetrahydrothiopyran-4-yl-3,5,7-triazabicyclo[4.4.0]deca-6,8,10-trien-3-yl)cyclohexyl]piperidine-2-carboxamide;
3-bromo-N-[4-(9-fluoro-2,4-dioxo-5-tetrahydrothiopyran-4-yl-3,5,7-triazabicyclo[4.4.0]deca-6,8,10-trien-3-yl)cyclohexyl]-1,7-diazabicyclo[4.3.0]nona-2,4,6,8-tetraene-8-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-[5-(trifluoromethyl)-2-pyridyl]piperidine-4-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]quinuclidine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-methyl-4-phenyl-piperidine-4-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-methylpiperazin-1-yl)pyridine-3-carboxamide;
1-butyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(p-tolyl)piperidine-4-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-9-oxa-2,8-diazaspiro[4.4]non-7-ene-7-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]carboxamide;
2-(1,4-diazepane-1-yl)-N-[4-(9-fluoro-2,4-dioxo-5-tetrahydrothiopyran-4-yl-3,5,7-triazabicyclo[4.4.0]deca-6,8,10-trien-3-yl)cyclohexyl]pyridine-3-carboxamide;
6-(1,4-diazepane-1-yl)-N-[4-(9-fluoro-2,4-dioxo-5-tetrahydrothiopyran-4-yl-3,5,7-triazabicyclo[4.4.0]deca-6,8,10-trien-3-yl)cyclohexyl]pyridine-3-carboxamide;
N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-6-(1-methylpyrrolidin-2-yl)imidazo[1,2-a]pyridine-2-carboxamide;
3-ethyl-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-pentanamide;
2-cyclopentyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-phenyl-acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-cyclopropane-1-carboxamide;
4-(4-chloro-2-methyl-phenoxy)-N-[4-(9-fluoro-2,4-dioxo-5-tetrahydrothiopyran-4-yl-3,5,7-triazabicyclo[4.4.0]deca-6,8,10-trien-3-yl)cyclohexyl]butanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methyl-2-phenyl-pentanamide;
4-(3,4-dimethoxyphenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]butanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-methyl-cyclohexane-1-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methoxy-cyclohexane-1-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-methylcyclohexyl)acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]tetrahydrofuran-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3,3-diphenyl-propanamide;
4,4,4-trifluoro-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methyl-butanamide;
(3R)—N-[4-(9-fluoro-2,4-dioxo-5-tetrahydrothiopyran-4-yl-3,5,7-triazabicyclo[4.4.0]deca-6,8,10-trien-3-yl)cyclohexyl]-3-phenyl-butanamide;
3-cyclopentyl-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide;
1-(4-chlorophenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]cyclopentane-1-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-phenyl-cyclopropane-1-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-(4-methoxyphenyl)cyclopropane-1-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-(p-tolyl)cyclopentane-1-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-tert-butyl-cyclohexane-1-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(2-methoxyphenyl)propanamide;
2-cyclopentyl-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-methyl-nonanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(p-tolyl)butanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methyl-2-phenyl-butanamide;
2-cyclopropyl-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(2-methyl-1-oxo-3-isoquinolyl)butanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-hydroxy-cyclohexane-1-carboxamide;
N′-benzyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-N′-(2-thienylmethyl)butanediamine;
2-(5-chloro-1-ethyl-benzoimidazol-2-yl)sulfanyl-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-hydroxy-3-tert-butyl-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2,3-dimethyl-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]isoxazole-5-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methylsulfanyl-acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-phenoxy-acetamide;
3-cyano-N-[4-(9-fluoro-2,4-dioxo-5-tetrahydrothiopyran-4-yl-3,5,7-triazabicyclo[4.4.0]deca-6,8,10-trien-3-yl)cyclohexyl]-3,3-diphenyl-propanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methoxy-acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(3-hydroxy-1-adamantyl)acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-1H-benzoimidazole-5-carboxamide;
(2R)—N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-2-phenyl-propanamide;
3-(3,4-dichlorophenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]-2,2-dimethyl-propanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(5-methylpyrazol-1-yl)acetamide;
1-acetyl-N-[4-(9-fluoro-2,4-dioxo-5-tetrahydrothiopyran-4-yl-3,5,7-triazabicyclo[4.4.0]deca-6,8,10-trien-3-yl)cyclohexyl]piperidine-3-carboxamide;
1-acetyl-N-[4-(9-fluoro-2,4-dioxo-5-tetrahydrothiopyran-4-yl-3,5,7-triazabicyclo[4.4.0]deca-6,8,10-trien-3-yl)cyclohexyl]pyrrolidine-2-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(4-pyridyl)benzamide;
2-(1H-benzoimidazol-2-ylsulfanyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]pentanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2,5-dimethoxy-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1H-benzoimidazole-5-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-pyridylsulfanyl)acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-5-isopropyl-3,8-dimethyl-2,4-dioxo-7-thia-3,5-diazabicyclo[4.3.0]nona-8,10-diene-9-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-isobutylphenyl)propanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-isopropoxy-4-methoxy-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-oxo-3H-benzothiazole-6-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methylsulfonyl-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(3-quinolyloxy)acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-quinoline-3-carboxamide;
2-ethoxy-N-[4-(9-fluoro-2,4-dioxo-5-tetrahydrothiopyran-4-yl-3,5,7-triazabicyclo[4.4.0]deca-6,8,10-trien-3-yl)cyclohexyl]pyridine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-[[5-(trifluoromethyl)-2-pyridyl]sulfonyl]propanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-6-methyl-pyridine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]pyridine-2-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]quinoline-2-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-isopropylsulfanyl-7,9-dimethyl-5,7,8-triazabicyclo[4.3.0]nona-1,3,5,8-tetraene-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methoxy-pyridine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(2,2,2-trifluoroethoxy)pyridine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(2-methyl-4-pyridyl)benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methoxy-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-9-phenyl-nonanamide;
(2S)—N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-2-phenyl-acetamide;
2-acetamido-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide;
2-(4-chlorophenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-propanamide;
N-[(1S)-1-[[4-(9-fluoro-2,4-dioxo-5-tetrahydrothiopyran-4-yl-3,5,7-triazabicyclo[4.4.0]deca-6,8,10-trien-3-yl)cyclohexyl]carbamoyl]-2-phenyl-ethyl]benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methoxy-propanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]naphthalene-1-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-phenyl-5-propyl-pyrazole-4-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2,4,6-trimethyl-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3,5,5-trimethyl-hexanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-hydroxy-2-phenyl-propanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-pentyl-benzamide;
2-cyclohexyl-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide;
2-cyclohexyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-phenyl-acetamide;
2-benzyl-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-5-phenyl-pentanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-propyl-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-6-methyl-3-tert-butyl-benzamide;
3-ethoxy-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide;
2-fluoro-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-fluorophenyl)propanamide;
3,4-dichloro-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1H-pyrazole-4-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-6-phenyl-hexanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-isopropyl-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-phenyl-butanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(4-methoxyphenyl)butanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]cyclopentanecarboxamide;
(2R)—N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methoxy-2-phenyl-acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-methylsulfonyl-benzamide;
4-benzyloxy-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-pentylsulfanyl-pyridine-3-carboxamide;
4-butoxy-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methylsulfanyl-pyridine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-phenoxy-pyridine-3-carboxamide;
4,4-difluoro-N-[4-(9-fluoro-2,4-dioxo-5-tetrahydrothiopyran-4-yl-3,5,7-triazabicyclo[4.4.0]deca-6,8,10-trien-3-yl)cyclohexyl]cyclohexane-1-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-5-methyl-1-phenyl-pyrazole-4-carboxamide;
(1R,2R)—N-[4-(9-fluoro-2,4-dioxo-5-tetrahydrothiopyran-4-yl-3,5,7-triazabicyclo[4.4.0]deca-6,8,10-trien-3-yl)cyclohexyl]-2-phenyl-cyclopropane-1-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-indan-2-yl-acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(p-tolylsulfonyl)pyridine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-methylphenoxy)pyridine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2,8-dimethyl-1,5,9-triazabicyclo[4.3.0]nona-2,4,6,8-tetraene-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methyl-furan-2-carboxamide;
2-(4,6-dimethylpyrimidin-2-yl)sulfanyl-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(2-oxobenzooxazol-3-yl)propanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-4-methoxy-benzamide;
2-(2-cyanophenyl)sulfanyl-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-hexoxy-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-octyl-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(hydroxymethyl)benzamide;
N′-[4-(9-fluoro-2,4-dioxo-5-tetrahydrothiopyran-4-yl-3,5,7-triazabicyclo[4.4.0]deca-6,8,10-trien-3-yl)cyclohexyl]-N-[(1S)-1-phenylethyl]phthalamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-5-methoxy-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3,4-dimethyl-benzamide;
3-fluoro-N-[4-(9-fluoro-2,4-dioxo-5-tetrahydrothiopyran-4-yl-3,5,7-triazabicyclo[4.4.0]deca-6,8,10-trien-3-yl)cyclohexyl]-4-methoxy-benzamide;
1-benzyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-oxo-pyridine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]butanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(p-tolylsulfonyl)acetamide;
4-butyl-N-[4-(9-fluoro-2,4-dioxo-5-tetrahydrothiopyran-4-yl-3,5,7-triazabicyclo[4.4.0]deca-6,8,10-trien-3-yl)cyclohexyl]cyclohexane-1-carboxamide;
2-(benzenesulfonyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(p-tolylsulfonylamino)acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-3-methyl-butanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(2-methoxyethoxy)benzamide;
2-(3,4-dimethoxyphenyl)sulfanyl-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-tetrahydropyran-4-yl-acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1,5-dimethyl-pyrazole-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-3-oxo-5-thia-2-azabicyclo[4.4.0]deca-6,8,10-triene-9-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-phenoxy-propanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-hydroxy-butanamide;
(1R,2S)—N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-cyclopentane-1-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(hydroxymethyl)-1H-pyrazole-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methoxy-3-methyl-benzamide;
3-ethoxy-N-[4-(9-fluoro-2,4-dioxo-5-tetrahydrothiopyran-4-yl-3,5,7-triazabicyclo[4.4.0]deca-6,8,10-trien-3-yl)cyclohexyl]-4-methoxy-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-tert-butyl-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2,3-dihydrobenzofuran-5-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-[4-(trifluoromethyl)phenoxy]acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-5-methyl-isoxazole-3-carboxamide;
4-ethylsulfanyl-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide;
6-chloro-2-fluoro-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methyl-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-8-methyl-1,7-diazabicyclo[4.3.0]nona-2,4,6,8-tetraene-9-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2,2-dimethyl-tetrahydropyran-4-carboxamide;
2-(3,5-dimethyl-1,2,4-triazol-1-yl)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(methylsulfonylmethyl)benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]tetrahydropyran-4-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-2-(3-hydroxyphenyl)acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(2-methoxyethoxy)acetamide;
4-(2,5-dioxabicyclo[4.4.0]deca-7,9,11-trien-9-yl)-N-[4-(9-fluoro-2,4-dioxo-5-tetrahydrothiopyran-4-yl-3,5,7-triazabicyclo[4.4.0]deca-6,8,10-trien-3-yl)cyclohexyl]butanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(3-methylisoxazol-5-yl)acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-(p-tolyl)cyclohexane-1-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-(4-methoxyphenyl)cyclopentane-1-carboxamide;
2-benzylsulfanyl-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-phenoxy-propanamide;
2-benzyloxy-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-hydroxyphenyl)propanamide;
2-(4-chlorophenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-[(4-methoxyphenyl)methylsulfanyl]-3-methyl-butanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(3-methylsulfanylphenyl)acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-methyl-5-oxo-pyrrolidine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-2-[4-(trifluoromethyl)phenyl]acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-[(1S,4R)-norbornane-2-yl]acetamide;
2-ethylsulfanyl-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2,3-dimethyl-butanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-phenyl-propanamide;
4-(1,1-dioxothiazinan-2-yl)-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide;
(2S)—N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-3-phenyl-propanamide;
3-ethyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-4-phenyl-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-5-(4-fluorophenyl)pentanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-5-phenyl-furan-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(1,1,2,2-tetrafluoroethoxy)benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-5-(2-phenylethynyl)furan-2-carboxamide;
2-cyclohexyl-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-5-(2-phenylethynyl)pyridine-3-carboxamide;
2-(5-chloro-2-fluoro-phenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide;
N-[2-[[4-(9-fluoro-2,4-dioxo-5-tetrahydrothiopyran-4-yl-3,5,7-triazabicyclo[4.4.0]deca-6,8,10-trien-3-yl)cyclohexyl]carbamoyl]ethyl]benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(3-methylpyrazol-1-yl)propanamide;
3-(3,4-dichlorophenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-phenethyloxy-propanamide;
(2R)-2-acetamido-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-phenyl-acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(p-tolyl)benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-[4-(trifluoromethyl)phenyl]benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-5-methyl-1H-imidazole-4-carboxamide;
5-ethyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-1-phenyl-pyrazole-4-carboxamide;
(2S)-2-acetamido-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-phenyl-propanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2,3,5,6-tetramethyl-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-hydroxy-3,5-dimethyl-benzamide;
3-(benzenesulfinyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-5-methyl-benzothiophene-2-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methyl-imidazole-4-carboxamide;
1-[(2-chlorophenyl)methyl]-N-[4-(9-fluoro-2,4-dioxo-5-tetrahydrothiopyran-4-yl-3,5,7-triazabicyclo[4.4.0]deca-6,8,10-trien-3-yl)cyclohexyl]-5-oxo-pyrrolidine-3-carboxamide;
(2S)—N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-4-methyl-pentanamide;
1-benzoyl-N-[4-(9-fluoro-2,4-dioxo-5-tetrahydrothiopyran-4-yl-3,5,7-triazabicyclo[4.4.0]deca-6,8,10-trien-3-yl)cyclohexyl]piperidine-4-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1H-benzoimidazole-2-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-4-phenyl-pyrimidine-5-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1,3,5-trimethyl-pyrazole-4-carboxamide;
4-(3,5-dimethylpyrazol-1-yl)-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-methyl-5-phenyl-pyrazole-4-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-5-phenyl-pyridine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-methyl-3-(trifluoromethyl)pyrazole-4-carboxamide;
3-(3,5-dimethyl-1H-pyrazol-4-yl)-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(2-methylbenzimidazol-1-yl)propanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-6-methoxy-benzothiazole-2-carboxamide;
1-benzyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-5-oxo-pyrrolidine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-isopropyl-pyrazole-4-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]heptanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-4-[2-(m-tolyl)ethyl]thiazole-5-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-methylsulfonyl-piperidine-4-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4,8-dimethyl-1,7-diazabicyclo[4.3.0]nona-2,4,6,8-tetraene-9-carboxamide;
6-ethyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-4-hydroxy-quinoline-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-isobutyl-3-oxo-isoindoline-4-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-[4-(tetrazol-1-yl)phenoxy]acetamide;
3-(2,4-dimethyl-1,5,7,9-tetrazabicyclo[4.3.0]nona-2,4,6,8-tetraene-3-yl)-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(2-hydroxyethyl)-1,5,9-triazabicyclo[4.3.0]nona-2,4,6,8-tetraene-8-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-10-oxo-1,7-diazabicyclo[4.4.0]deca-2,4,6,8-tetraene-9-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(5-methyltetrazole-1-yl)benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2,4-dimethyl-1,5,7,8-tetrazabicyclo[4.3.0]nona-2,4,6,8-tetraene-9-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1,5,9-triazabicyclo[4.3.0]nona-2,4,6,8-tetraene-8-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(o-tolyl)acetamide;
2-(4-chlorophenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide;
2-(2-benzyloxyphenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-methoxyphenyl)acetamide;
2-benzo[1,3]dioxol-5-yl-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide;
2-(3,5-dimethylphenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-5-methoxy-benzofuran-2-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-(2-hydroxyphenyl)-5-oxo-pyrrolidine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-methylsulfonylphenyl)acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(2-naphthyl)acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(2-phenethylsulfanylethylsulfanyl)acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-[2-(2-pyridyl)pyrrolidin-1-yl]pyridine-3-carboxamide;
2-(4-butoxyphenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide;
2-(2,4-dimethylthiazol-5-yl)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide;
N-[4-(9-fluoro-2,4-dioxo-5-tetrahydrothiopyran-4-yl-3,5,7-triazabicyclo[4.4.1]deca-6,8,10-trien-3-yl)cyclohexyl]-2-(4-methylphenoxy)propanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-fluorophenoxy)propanamide;
3-benzothiazol-2-yl-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(2-pyridyloxy)acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-4-phenyl-thiazole-5-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(4-fluorophenyl)propanamide;
2-fluoro-N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(5-fluoro-2-methoxy-phenyl)benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methoxy-5-(trifluoromethyl)benzamide;
3-(4-chlorophenyl)sulfanyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-1H-indole-5-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-naphthalene-1-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(5-methyl-2-phenyl-thiazol-4-yl)acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-hexyl-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-butanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-6-oxo-2-(1,1,2,2,2-pentafluoroethyl)-1H-pyridine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-5-isobutyl-3-methyl-2,4-dioxo-8-[[2-(trifluoromethyl)phenyl]methyl]-3,5,7,8-tetrazabicyclo[4.3.0]nona-6,9-diene-9-carboxamide;
2-chloro-N-[4-(9-fluoro-2,4-dioxo-5-tetrahydrothiopyran-4-yl-3,5,7-triazabicyclo[4.4.0]deca-6,8,10-trien-3-yl)cyclohexyl]-6-methyl-benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]dodecanamide;
(3R)—N-[4-(9-fluoro-2,4-dioxo-5-tetrahydrothiopyran-4-yl-3,5,7-triazabicyclo[4.4.0]deca-6,8,10-trien-3-yl)cyclohexyl]-5-oxo-1-[(1R)-1-phenylethyl]pyrrolidine-3-carboxamide;
(2R)-3,3,3-trifluoro-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-2-methyl-propanamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1-(2-methoxyphenyl)-5-oxo-pyrrolidine-3-carboxamide;
3,3,3-trifluoro-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-propanamide;
2,6-diethyl-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(2-methoxyethoxy)benzamide;
(3R)-1-[(3,4-dichlorophenyl)methyl]-N-[4-(9-fluoro-2,4-dioxo-5-tetrahydrothiopyran-4-yl-3,5,7-triazabicyclo[4.4.0]deca-6,8,10-trien-3-yl)cyclohexyl]piperidine-3-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-1,5,9-triazabicyclo[4.3.0]nona-2,4,6,8-tetraene-7-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-hydroxyphenoxy)acetamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]indane-2-carboxamide;
(2S,5R)-1-butyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-5-(4-methoxyphenyl)pyrrolidine-2-carboxamide;
N-[4-(9-fluoro-2,4-dioxo-5-tetrahydrothiopyran-4-yl-3,5,7-triazabicyclo[4.4.0]deca-6,8,10-trien-3-yl)cyclohexyl]isochroman-1-carboxamide;
2-ethyl-N-[4-(9-fluoro-2,4-dioxo-5-tetrahydrothiopyran-4-yl-3,5,7-triazabicyclo[4.4.0]deca-6,8,10-trien-3-yl)cyclohexyl]hexanamide;
(2R)—N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-hydroxyphenoxy)propanamide;
(2S)—N-[4-(9-fluoro-2,4-dioxo-5-tetrahydrothiopyran-4-yl-3,5,7-triazabicyclo[4.4.0]deca-6,8,10-trien-3-yl)cyclohexyl]-5-oxo-pyrrolidine-2-carboxamide; or,
(2S)-2-acetamido-N-[4-(9-fluoro-2,4-dioxo-5-tetrahydrothiopyran-4-yl-3,5,7-triazabicyclo[4.4.0]deca-6,8,10-trien-3-yl)cyclohexyl]-4-methyl-pentanamide;
   or a pharmaceutically acceptable salt thereof.

The compounds of the present invention can be prepared as described below or by adapting methods known in the art.

In a further aspect the invention provides a process for the preparation of a compound of formula (I), which comprises removing the Boc protecting group from a compound of formula (II)

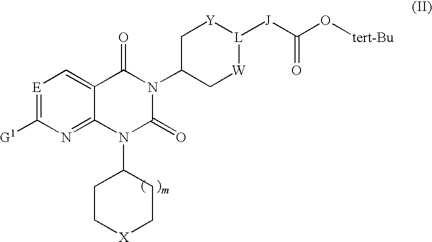

wherein m, G¹, E, Y, L, W, X and J are as defined in formula (I), (for example with an acid such as trifluoroacetic acid or hydrochloric acid) and reacting the product so formed with a carboxylic acid of formula (III):

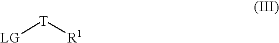

wherein R¹ and T are as defined in formula (I), and LG is a represents a leaving group. The process is carried out at a suitable temperature, generally between 0° C. and the boiling point of the solvent, in a suitable solvent such as dichloromethane or N-methylpyrrolidinone. The process is optionally carried out in the presence of a base and/or a coupling reagent such as HATU, HOAT, HOBT, DIEA or T₃P (2,4,6-tripropyl-1,3,5,2,4,6-trioxatriphosphinane 2,4,6-trioxide). Suitable leaving groups LG include OH and halogen, particularly OH.

A compound of formula (II) wherein m, G¹, E, Y, L, W, and J are as defined in formula (I), can be prepared by condensing a compound of formula (IV):

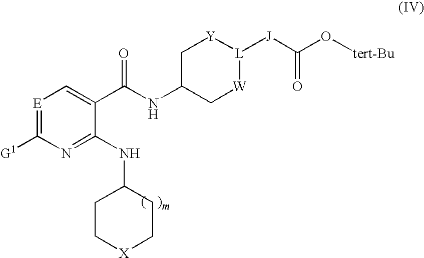

wherein m, G¹, E, Y, L, W, X and J are as defined in formula (I), with a suitable carbonylating agent such as carbonyl diimidazole or ethyl chlorformate in the presence of a suitable base such as sodium hydride. The process is carried out at a suitable temperature, generally between 0° C. and the boiling point of the solvent, in a suitable solvent such as tetrahydrofuran.

A compound of formula (IV) wherein m, G¹, E, Y, L, W, T, and J are as defined in formula (I), can be prepared by reacting a compound of formula (V):

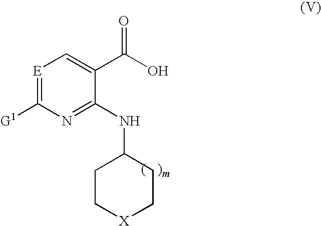

wherein m, X, G¹ and E are as defined in formula (I), with an amine of formula (VI)

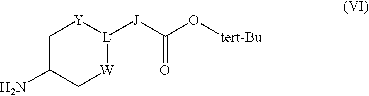

wherein Y, L, W, and J are as defined in formula (I). The process is carried out at a suitable temperature, generally between 0° C. and the boiling point of the solvent, in a suitable solvent such as dichloromethane. The process is optionally carried out in the presence of a base and a coupling reagent such as HATU, HOAT, HOBT, DIEA or T₃P (2,4,6-tripropyl-1,3,5,2,4,6-trioxatriphosphinane 2,4,6-trioxide).

A compound of formula (V) wherein m, G¹ and E are as defined in formula (I), can be prepared by reacting a compound of formula (VII):

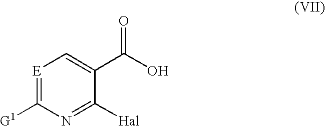

wherein G¹ and E are as defined in formula (I) and Hal represents a halogen atom, with 4-amino-thiopyran, 3-aminotetrahydrothiophen or an S-oxide or S-dioxide of either of these rings. The process is carried out at a suitable temperature, generally between 50° C. and the boiling point of the solvent, in a suitable solvent such as dimethylformamide. The process is optionally carried out in the presence of a base such as potassium carbonate.

The preparations of various intermediates are described in the literature or can be prepared by routine adaptation of methods described in the literature.

In the above processes it may be desirable or necessary to protect an acid group or a hydroxy or other potentially reactive group. Suitable protecting groups and details of processes for adding and removing such groups may be found in “Protective Groups in Organic Synthesis”, 3rd Edition (1999) by Greene and Wuts.

In another aspect the present invention provides processes for the preparation of compounds of formula (I).

The compounds of formula (I) have activity as pharmaceuticals, in particular as modulators of PDE 4 receptor activity, and may be used in the treatment of inflammatory diseases, asthma or COPD.

Examples of disease states that can be treated with a compound of the invention are:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) or adenovirus; or eosinophilic esophagitis;
2. bone and joints: arthritides associated with or including osteoarthritis/osteoarthrosis, both primary and secondary to, for example, congenital hip dysplasia; cervical and lumbar spondylitis, and low back and neck pain; osteoporosis; rheumatoid arthritis and Still's disease; seronegative spondyloarthropathies including ankylosing spondylitis, psoriatic arthritis, reactive arthritis and undifferentiated spondarthropathy; septic arthritis and other infection-related arthropathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositis and polymyositis; polymyalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthralgias, tendonititides, and myopathies;
3. pain and connective tissue remodelling of musculoskeletal disorders due to injury [for example sports injury] or disease: arthritides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritis, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosis et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiform; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; esophagitis, including reflux; eosinophilic gastro-enteritis, mastocytosis, Crohn's disease, colitis including ulcerative colitis, proctitis, pruritus ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopenic purpura, eosinophilic fascitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; or,
15. gastrointestinal tract: Coeliac disease, proctitis, eosinophilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

According to a further feature of the present invention there is provided a method for treating a PDE 4 mediated disease state in a mammal, such as man, suffering from, or at risk of, said disease state, which comprises administering to a mammal in need of such treatment a therapeutically effective amount of a compound of the formula (I) or a pharmaceutically acceptable salt thereof.

The invention also provides a compound of the formula (I), or a pharmaceutically acceptable salt thereof, for use in therapy.

In another aspect the invention provides the use of a compound of formula (I), or a pharmaceutically acceptable salt thereof, in the manufacture of a medicament for use in therapy (for example modulating PDE 4 enzymatic activity).

The invention further provides the use of a compound of formula (I), or a pharmaceutically acceptable salt thereof, in the manufacture of a medicament for use in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) or adenovirus; or eosinophilic esophagitis;
2. bone and joints: arthritides associated with or including osteoarthritis/osteoarthrosis, both primary and secondary to, for example, congenital hip dysplasia; cervical and lumbar spondylitis, and low back and neck pain; osteoporosis; rheumatoid arthritis and Still's disease; seronegative spondyloarthropathies including ankylosing spondylitis, psoriatic arthritis, reactive arthritis and undifferentiated spondarthropathy; septic arthritis and other infection-related arthropathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositis and polymyositis; polymyalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthralgias, tendonititides, and myopathies;
3. pain and connective tissue remodelling of musculoskeletal disorders due to injury [for example sports injury] or disease: arthritides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritis, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosis et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiform; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; esophagitis, including reflux; eosinophilic gastro-enteritis, mastocytosis, Crohn's disease, colitis including ulcerative colitis, proctitis, pruritus ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopenic purpura, eosinophilic fascitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; or,
15. gastrointestinal tract: Coeliac disease, proctitis, eosinophilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema;
in a mammal (for example man).

In a further aspect the invention provides a compound of formula (I), or a pharmaceutically acceptable salt thereof, for use in the treatment of asthma {such as bronchial, allergic, intrinsic, extrinsic or dust asthma, particularly chronic or inveterate asthma (for example late asthma or airways hyper-responsiveness)}; or COPD.

In a still further aspect a compound of formula (I), or a pharmaceutically acceptable salt thereof, is useful in the treatment of COPD.

The present invention also provides the use of a compound of formula (I), or a pharmaceutically acceptable salt thereof, in the manufacture of a medicament for use in the treatment of asthma {such as bronchial, allergic, intrinsic, extrinsic or dust asthma, particularly chronic or inveterate asthma (for example late asthma or airways hyper-responsiveness)}; or COPD.

In order to use a compound of the invention, or a pharmaceutically acceptable salt thereof, for the therapeutic treatment of a mammal, such as man, said ingredient is normally formulated in accordance with standard pharmaceutical practice as a pharmaceutical composition. Therefore in another aspect the present invention provides a pharmaceutical composition which comprises a compound of the formula (I), or a pharmaceutically acceptable salt thereof (active ingredient), and a pharmaceutically acceptable adjuvant, diluent or carrier.

In a further aspect the present invention provides a process for the preparation of said composition which comprises mixing active ingredient with a pharmaceutically acceptable adjuvant, diluent or carrier. Depending on the mode of administration, the pharmaceutical composition will, for example, comprise from 0.05 to 99% w (percent by weight), such as from 0.05 to 80% w, for example from 0.10 to 70% w, such as from 0.10 to 50% w, of active ingredient, all percentages by weight being based on total composition.

The pharmaceutical compositions of this invention may be administered in standard manner for the disease condition that it is desired to treat, for example by topical (such as to the lung and/or airways or to the skin), inhalation, oral, rectal or parenteral administration. For these purposes the compounds of this invention may be formulated by means known in the art. A suitable pharmaceutical composition of this invention is one suitable for oral administration in unit dosage form, for example a tablet or capsule which contains between 0.1 mg and 1 g of active ingredient.

Each patient may receive, for example, a dose of 0.001 mgkg⁻¹ to 100 mgkg⁻¹, for example in the range of 0.1 mgkg⁻¹ to 20 mgkg⁻¹, of the active ingredient administered, for example, 1 to 4 times per day.

The invention further relates to a combination therapy wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with agents listed below.

Non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerin; intra-articular therapies such as hyaluronic acid derivatives; and nutritional supplements such as glucosamine.

The present invention still further relates to the combination of a compound of the invention, or a pharmaceutically acceptable salt thereof, together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxifylline.

In addition the invention relates to a combination of a compound of the invention, or a pharmaceutically acceptable salt thereof, with a monoclonal antibody targeting B-Lymphocytes (such as CD20 (rituximab), MRA-aIL16R and T-Lymphocytes, CTLA4-Ig, HuMax I1-15).

The present invention still further relates to the combination of a compound of the invention, or a pharmaceutically acceptable salt thereof, with a modulator of chemokine receptor function such as an antagonist of CCR1, CCR2, CCR2A, CCR2B, CCR3, CCR4, CCR5, CCR6, CCR7, CCR8, CCR9, CCR10 and CCR1 (for the C—C family); CXCR1, CXCR2, CXCR3, CXCR4 and CXCR5 (for the C—X—C family) and CX₃CR1 for the C—X₃—C family.

The present invention further relates to the combination of a compound of the invention, or a pharmaceutically acceptable salt thereof, with an inhibitor of matrix metalloprotease (MMPs), i.e., the stromelysins, the collagenases, and the gelatinases, as well as aggrecanase; for example collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention, or a pharmaceutically acceptable salt thereof, and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY x 1005.

The present invention further relates to the combination of a compound of the invention, or a pharmaceutically acceptable salt thereof, and a receptor antagonist for leukotrienes (LT) B4, LTC4, LTD4, and LTE4. selected from the group consisting of the phenothiazine-3-yls such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention, or a pharmaceutically acceptable salt thereof, and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention, or a pharmaceutically acceptable salt thereof, and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention, or a pharmaceutically acceptable salt thereof, and a proton pump inhibitor (such as omeprazole) or a gastroprotective histamine type 2 receptor antagonist.

The present invention further relates to the combination of a compound of the invention, or a pharmaceutically acceptable salt thereof, and an antagonist of the histamine type 4 receptor.

The present invention still further relates to the combination of a compound of the invention, or a pharmaceutically acceptable salt thereof, and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention, or a pharmaceutically acceptable salt thereof, and an anticholinergic agent including muscarinic receptor (M1, M2, and M3) antagonist such as atropine, hyoscine, glycopyrrrolate, ipratropium bromide, tiotropium bromide, oxitropium bromide, pirenzepine or telenzepine.

The present invention still further relates to the combination of a compound of the invention, or a pharmaceutically acceptable salt thereof, and a beta-adrenoceptor agonist (including beta receptor subtypes 1-4) such as isoprenaline, salbutamol, formoterol, salmeterol, terbutaline, orciprenaline, bitolterol mesylate, or pirbuterol, or a chiral enantiomer thereof.

The present invention further relates to the combination of a compound of the invention, or a pharmaceutically acceptable salt thereof, and a chromone, such as sodium cromoglicate or nedocromil sodium.

The present invention still further relates to the combination of a compound of the invention, or a pharmaceutically acceptable salt thereof, with a glucocorticoid, such as flunisolide, triamcinolone acetonide, beclomethasone dipropionate, budesonide, fluticasone propionate, ciclesonide or mometasone furoate.

The present invention further relates to the combination of a compound of the invention, or a pharmaceutically acceptable salt thereof, with an agent that modulates a nuclear hormone receptor such as PPARs.

The present invention still further relates to the combination of a compound of the invention, or a pharmaceutically acceptable salt thereof, together with an immunoglobulin (Ig) or Ig preparation or an antagonist or antibody modulating Ig function such as anti-IgE (for example omalizumab).

The present invention further relates to the combination of a compound of the invention, or a pharmaceutically acceptable salt thereof, and another systemic or topically-applied anti-inflammatory agent, such as thalidomide or a derivative thereof, a retinoid, dithranol or calcipotriol.

The present invention still further relates to the combination of a compound of the invention, or a pharmaceutically acceptable salt thereof, and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a compound of the invention, or a pharmaceutically acceptable salt thereof, together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention, or a pharmaceutically acceptable salt thereof, and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxifylline; thrombolytic, or an anticoagulant such as a platelet aggregation inhibitor.

The present invention further relates to the combination of a compound of the invention, or a pharmaceutically acceptable salt thereof, and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention, or a pharmaceutically acceptable salt thereof, and an agent for the treatment of acute or chronic pain, such as a centrally or peripherally-acting analgesic (for example an opioid or derivative thereof), carbamazepine, phenyloin, sodium valproate, amitryptyline or other anti-depressant agents, paracetamol, or a non-steroidal anti-inflammatory agent.

The present invention further relates to the combination of a compound of the invention, or a pharmaceutically acceptable salt thereof, together with a parenterally or topically-applied (including inhaled) local anaesthetic agent such as lignocaine or a derivative thereof.

A compound of the present invention, or a pharmaceutically acceptable salt thereof, can also be used in combination with an anti-osteoporosis agent including a hormonal agent such as raloxifene, or a biphosphonate such as alendronate.

The present invention still further relates to the combination of a compound of the invention, or a pharmaceutically acceptable salt thereof, together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B.sub1.- or B.sub2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK.sub1. or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of p38; (xxv) agent modulating the function of Toll-like receptors (TLR), (xxvi) agent modulating the activity of purinergic receptors such as P2×7; (xxvii) inhibitor of transcription factor activation such as NFkB, API, or STATS; or (xxviii) a glucocorticoid receptor (GR-receptor) agonist.

In a further embodiment the present invention provides a pharmaceutical product comprising, in combination, a first active ingredient which is a compound of formula (I), or a pharmaceutically acceptable salt thereof, as hereinbefore described, and at least one further active ingredient selected from:—

a β2. adrenoceptor agonist,

a modulator of chemokine receptor function,

an inhibitor of kinase function,

a protease inhibitor,

a steroidal glucocorticoid receptor agonist,

an anticholinergic agent, and a

a non-steroidal glucocorticoid receptor agonist.

The pharmaceutical product according to this embodiment may, for example, be a pharmaceutical composition comprising the first and further active ingredients in admixture. Alternatively, the pharmaceutical product may, for example, comprise the first and further active ingredients in separate pharmaceutical preparations suitable for simultaneous, sequential or separate administration to a patient in need thereof. The pharmaceutical product of this embodiment is of particular use in treating respiratory diseases such as asthma, COPD or rhinitis.

Examples of a β₂-adrenoceptor agonist that may be used in the pharmaceutical product according to this embodiment include metaproterenol, isoproterenol, isoprenaline, albuterol, salbutamol (e.g. as sulphate), formoterol (e.g. as fumarate), salmeterol (e.g. as xinofoate), terbutaline, orciprenaline, bitolterol (e.g. as mesylate), pirbuterol or indacaterol. The β₂-adrenoceptor agonist of this embodiment may be a long-acting β₂-agonists, for example salmeterol (e.g. as xinofoate), formoterol (e.g. as fumarate), bambuterol (e.g. as hydrochloride), carmoterol (TA 2005, chemically identified as 2(1H)-Quinolone, 8-hydroxy-5-[1-hydroxy-2-[[2-(4-methoxy-phenyl)-1-methylethyl]-amino]ethyl]-monohydrochloride, [R—(R*,R*)] also identified by Chemical Abstract Service Registry Number 137888-11-0 and disclosed in U.S. Pat. No. 4,579,854), indacaterol (CAS no 312753-06-3; QAB-149), formanilide derivatives e.g. 3-(4-{[6-({(2R)-2-[3-(formylamino)-4-hydroxyphenyl]-2-hydroxyethyl}amino)hexyl]oxy}-butyl)-benzenesulfonamide as disclosed in WO 2002/76933, benzenesulfonamide derivatives e.g. 3-(4-{[6-({(2R)-2-hydroxy-2-[4-hydroxy-3-(hydroxy-methyl)phenyl]ethyl}amino)-hexyl]oxy}butyl)benzenesulfonamide as disclosed in WO 2002/88167, aryl aniline receptor agonists as disclosed in WO 2003/042164 and WO 2005/025555, indole derivatives as disclosed in WO 2004/032921 and US 2005/222144, and compounds GSK 159797, GSK 159802, GSK 597901, GSK 642444 and GSK 678007.

Examples of a modulator of chemokine receptor function that may be used in the pharmaceutical product according to this embodiment include a CCR1 receptor antagonist.

Examples of an inhibitor of kinase function that may be used in the pharmaceutical product according to this embodiment include a p38 kinase inhibitor and an IKK inhibitor.

Examples of a protease inhibitor that may be used in the pharmaceutical product according to this embodiment include an inhibitor of neutrophil elastase or an inhibitor of MMP12.

Examples of a steroidal glucocorticoid receptor agonist that may be used in the pharmaceutical product according to this embodiment include budesonide, fluticasone (e.g. as propionate ester), mometasone (e.g. as furoate ester), beclomethasone (e.g. as 17-propionate or 17,21-dipropionate esters), ciclesonide, loteprednol (as e.g. etabonate), etiprednol (as e.g. dicloacetate), triamcinolone (e.g. as acetonide), flunisolide, zoticasone, flumoxonide, rofieponide, butixocort (e.g. as propionate ester), prednisolone, prednisone, tipredane, steroid esters e.g. 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl)ester and 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, steroid esters according to DE 4129535, steroids according to WO 2002/00679, WO 2005/041980, or steroids GSK 870086, GSK 685698 and GSK 799943.

Examples of an anticholinergic agent that may be used in the pharmaceutical product according to this embodiment include for example a muscarinic receptor antagonist (for example a M1, M2 or M3 antagonist, such as a M3 antagonist) for example ipratropium (e.g. as bromide), tiotropium (e.g. as bromide), oxitropium (e.g. as bromide), tolterodine, pirenzepine, telenzepine, glycopyrronium bromide (such as R,R-glycopyrronium bromide or a mixture of R,S- and S, R-glycopyrronium bromide); mepensolate (e.g. as bromide), a quinuclidine derivative such as 3(R)-(2-hydroxy-2,2-dithien-2-ylacetoxy)-1-(3-phenoxypropyl)-1-azonia-bicyclo[2.2.2]octane bromide as disclosed in US 2003/0055080, quinuclidine derivatives as disclosed in WO 2003/087096 and WO 2005/115467 and DE 10050995; or GSK 656398 or GSK 961081.

Examples of a modulator of a non-steroidal glucocorticoid receptor agonist that may be used in the pharmaceutical product according to this embodiment include those described in WO2006/046916.

A compound of the invention, or a pharmaceutically acceptable salt thereof, can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antiestrogen (for example tamoxifen, toremifene, raloxifene, droloxifene or iodoxyfene), an estrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erb b2 antibody trast zumab, or the anti-erb b1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);
(vi) a vascular damaging agent such as combretastatin A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or,
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The invention will now be illustrated by the following non-limiting examples in which, unless stated otherwise:

(i) when given, ¹H NMR data is quoted and is in the form of delta values for major diagnostic protons, given in parts per million (ppm) relative to tetramethylsilane (TMS) as an internal standard, determined at 300 MHz or 400 MHz using perdeuterio DMSO-D6 (CD₃SOCD₃) or CDCl₃ as the solvent unless otherwise stated;
(ii) mass spectra (MS) were run with an electron energy of 70 electron volts in the chemical ionisation (CI) mode using a direct exposure probe. Where indicated ionisation was effected by electrospray ionisation (ES), or atmospheric pressure chemical ionisation (APCI), or multimode ionisation, a combination of ES ionisation and APCI. Where values for m/z are given, generally only ions which indicate the parent mass are reported, and the mass ions quoted are the positive or negative mass ions: [M]⁺, [M+H]⁺ or [M−H]⁻;
(iii) the title and sub-title compounds of the examples and methods were named using the index name program from Advanced Chemistry Development Inc, version 8.00 or were named using the IUPAC name program from Openeye and stereochemical descriptors added by hand. (See www.evesopen.com/products/applications/ogham.html)
(iv) unless stated otherwise, reverse phase HPLC was conducted using a Symmetry™, NovaPak™ or Xterra™ reverse phase silica column, all available from Waters Corp.
(v) unless stated otherwise, the following method was used for LC analysis:
Method A: Instrument Agilent 1100, Column: Kromasil C 8 100×3 mm, 5μ particle size, Solvent A: 0.1% TFA/water, Solvent B: 0.08% TFA/acetonitrile Flow: 1 mL/min, Gradient 10-100%/B 20 min, 100% B 1 min. Absorption was measured at 220, 254 and 280 nm. Method B: Instrument Agilent 1100, Column: XTerra C 8, 100×3 mm, 5μ particle size, Solvent A: 15 mM NH3/water, Solvent B: acetonitrile Flow: 1 mL/min, Gradient 10-100%/B 20 min, 100% B 1 min. Absorption was measured at 220, 254 and 280 nm;

| | |
|---|---|
| DMF | N,N-Dimethylformamide |
| NMP | 1-N-Methyl-2-pyrrolidinone |
| HOAT | 1-Hydroxy-7-azabenzotriazole |
| DIEA | N,N-Diisopropylethylamine |
| HATU | O-(7-Benzotriazol-1-yl)-N,N,N′,N′- |
| | tetramethyluronium hexafluorophosphate |
| THF | Tetrahydrofuran |
| DCM | Dichloromethane |
| BOC | tert-butoxycarbonyl |
| HPLC | High pressure liquid chromatography |
| d | Day(s) |
| h | Hour(s) |
| min | Minute(s) |

The starting materials for the Examples below are either commercially available or readily prepared by standard methods from known starting materials. For example, the following reactions are an illustration of the preparation of some of the starting materials.

### Preparation 1

### 5-Fluoro-2-(tetrahydro-2H-thiopyran-4-ylamino)nicotinic Acid

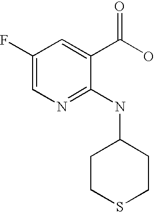

2-Chloro-5-fluoronicotinic acid (5.27 g, 30 mmol), K₂CO₃ (5 g, 36 mmol) was added to dry DMF (30 ml) under argon atmosphere. Copper (95 mg, 1.8 mmol), methanol-washed dried copper(I)bromide (215 mg, 1.5 mmol) and tetrahydro-2H-thiopyran-4-amine (6 g, 51 mmol) were added and the mixture was stirred at 150° C. for four hours. Ethyl acetate was added and the crude product was washed twice with 0.5 M aqueous citric acid, the organic solvents dried over Na₂SO₄, filtrated and removed in vacuum to afford the title compound (6 g, 78%).

¹H NMR (400 MHz, DMSO-d₆): δ 13.43 (1H, brs); 8.31 (1H, d); 7.97 (1H, brd); 7.91 (1H, dd); 3.99 (1H, brs); 2.76-2.61 (4H, m); 2.21 (2H, m); 1.64-1.53 (2H, m).

APCI-MS m/z: 257 [MH⁺].

### Preparation 2

### tert-Butyl [cis-4-({[5-fluoro-2-(tetrahydro-2H-thiopyran-4-ylamino)pyridin-3-yl]carbonyl}amino)cyclohexyl]carbamate

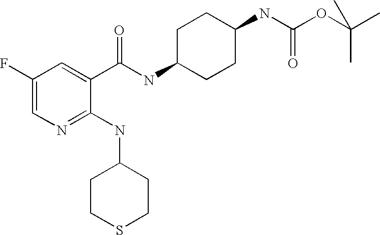

A mixture of 5-fluoro-2-(tetrahydro-2H-thiopyran-4-ylamino)nicotinic acid (5.9 g, 23 mmol), tert-butyl (cis-4-aminocyclohexyl)carbamate (5.42 g, 25.3 mmol), HATU (9.6 g, 25.3 mmol), HOAT (3.44 g, 25.3 mmol) and DIEA (12 ml, 70 mmol) in NMP (100 ml) was stirred for 10 min at room temperature (at pH 8-9 adjusted with DIEA). Ethyl acetate was added and the crude product was washed twice with 0.5 M aqueous citric acid, aqueous sodium hydrogencarbonate and water. The organic solvents dried over Na₂SO₄, filtrated and removed in vacuum. The residue was purified by flash chromatography on silica using ethyl acetate/heptane (1:3) as eluent to give the title compound (8.45 g, 81%).

¹H NMR (400 MHz, DMSO-d₆): δ 8.22 (1H, d); 8.17 (1H, brd); 8.16 (1H, brd); 7.96 (1H, dd); 6.61 (1H, brs); 3.92 (1H, brs); 3.77 (1H, brs); 3.40 (1H, brs); 2.74-2.60 (4H, m); 2.18 (2H, m); 1.70 (4H, m); 1.54 (6H, m); 1.39 (9H, s).

APCI-MS m/z: 453 [MH⁺].

LC (method A) rt=11.6 min

### Preparation 3

### tert-Butyl {cis-4-[6-fluoro-2,4-dioxo-1-(tetrahydro-2H-thiopyran-4-yl)-1,4-dihydropyrido[2,3-d]pyrimidin-3(2H)-yl]cyclohexyl}carbamate

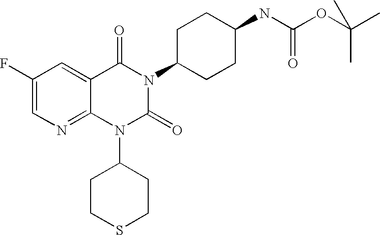

To a solution tert-butyl [cis-4-({[5-fluoro-2-(tetrahydro-2H-thiopyran-4-ylamino)pyridin-3-yl]carbonyl}amino)cyclohexyl]carbamate (3.9 g, 8.58 mmol) and 1,1′-carbonyldiimidazole (4.17 g, 25.7 mmol) in dry NMP (100 ml) under argon atmosphere 50% NaH in oil (1.24 g, 25.7 mmol) was added in 10 portions during one hour. The mixture was stirred at room temperature for two days. Ethyl acetate was added and the crude product was washed twice with 0.5 M aqueous citric acid, aqueous sodium hydrogencarbonate, and water. The organic solvents dried over Na₂SO₄, filtrated and removed in vacuum. The residue was purified by flash chromatography on silica using ethyl acetate/heptane (1:4) as eluent to give the title compound (1.95 mg, 48%) and 0.9 g (23%) of the starting material was recovered.

¹H NMR (400 MHz, DMSO-d₆): δ 8.78 (1H, d); 8.21 (1H, dd); 6.58 (1H, brs); 5.20 (1H, brs); 4.72 (1H, brt); 3.56 (1H, brs); 2.84-2.68 (6H, m); 2.58 (2H, q); 2.01-1.86 (4H, m); 1.49 (2H, brt); 1.42 (1H, brs).

APCI-MS m/z: 379 [MH⁺−tBOC].

LC (method A) rt=14.8 min

### Preparation 4

### 3-(cis-4-Aminocyclohexyl)-6-fluoro-1-(tetrahydro-2H-thiopyran-4-yl)pyrido[2,3-d]pyrimidine-2,4(1H,3H)-dione Hydrochloride

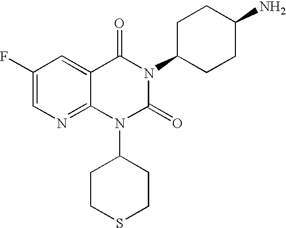

A mixture of tert-butyl {cis-4-[6-fluoro-2,4-dioxo-1-(tetrahydro-2H-thiopyran-4-yl)-1,4-dihydropyrido[2,3-d]pyrimidin-3(2H)-yl]cyclohexyl}carbamate (0.36 g, 0.75 mmol) and 4 M HCl in 1,4-Dioxane (10 ml) was stirred at room temperature for one hour. The solvents were removed and the pure crude product was used directly.

APCI-MS m/z: 379 [MH⁺].

### Preparation 5

### 5-Fluoro-2-(tetrahydro-3-thienylamino)nicotinic Acid

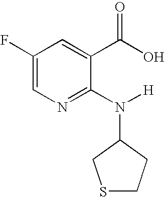

2-Chloro-5-fluoronicotinic acid (3.51 g, 20 mmol), K₂CO₃ (3.32 g, 24 mmol) was added to dry DMF (20 ml) under argon atmosphere. Copper (76 mg, 1.2 mmol), methanol-washed dried copper(I)bromide (144 mg, 1 mmol) and tetrahydrothiophene-3-amine (3.1 g, 30 mmol) were added and the mixture was stirred at 150° C. for four hours. Ethyl acetate was added and the crude product was washed twice with 0.5 M aqueous citric acid, the organic solvents dried over Na₂SO₄, filtrated and removed in vacuum to afford the title compound (3.98 g, 82%).

¹H NMR (400 MHz, DMSO-d₆): δ 13.5 (1H, brs); 8.35 (1H, d); 8.10 (1H, brs); 7.92 (1H, dd); 4.75 (1H, qv); 3.1 (1H, dd); 2.90 (1H, m); 2.82 (1H, m); 2.71 (1H, m); 2.06 (2H, m).

APCI-MS m/z: 243 [MH⁺].

### Preparation 6

### tert-Butyl[cis-4-({[5-fluoro-2-(tetrahydro-3-thienylamino)pyridin-3-yl]carbonyl}amino)cyclohexyl]carbamate

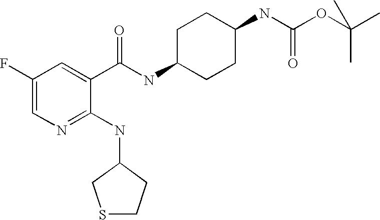

A mixture of 5-fluoro-2-(tetrahydro-3-thienylamino)nicotinic acid (3.98 g, 16.4 mmol), tert-butyl (cis-4-aminocyclohexyl)carbamate (3.87 g, 18.1 mmol), HATU (6.9 g, 18.1 mmol), HOAT (2.46 g, 18.1 mmol) and DIEA (8.6 ml, 50 mmol) in NMP (70 ml) was stirred for 1 hour at room temperature (at pH 8-9 adjusted with DIEA). Ethyl acetate was added and the crude product was washed twice with 0.5 M aqueous citric acid, aqueous sodium hydrogencarbonate and water. The organic solvents dried over Na₂SO₄, filtrated and removed in vacuum. The residue was purified by flash chromatography on silica using ethyl acetate/heptane (1:3) as eluent to give the title compound (3.95 g, 55%).

¹H NMR (400 MHz, DMSO-d₆): δ 8.34 (1H, d); 8.21 (1H, d); 8.19 (1H, brd); 7.98 (1H, dd); 6.61 (1H, brs); 4.68 (1H, m); 3.77 (1H, brs); 3.40 (1H, brs); 3.08 (1H, dd); 2.91-2.77 (2H, m); 2.65 (1H, dd); 2.02 (2H, m); 1.70 (4H, m); 1.52 (4H, m); 1.39 (9H, s).

APCI-MS m/z: 439 [MH⁺].

LC (method A) rt=11.6 min

### Preparation 7

### tert-Butyl {cis-4-[6-fluoro-2,4-dioxo-1-(tetrahydro-3-thienyl)-1,4-dihydropyrido[2,3-d]pyrimidin-3(2H)-yl]cyclohexyl}carbamate

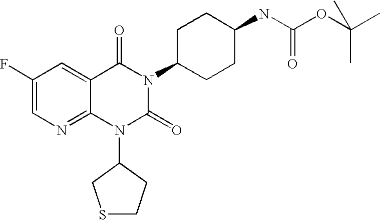

To a solution tert-butyl [cis-4-({[5-fluoro-2-(tetrahydro-3-thienylamino)pyridin-3-yl]carbonyl}amino)cyclohexyl]carbamate (2.2 g, 5 mmol) and 1,1′-carbonyldiimidazole (2.43 g, 15 mmol) in dry NMP (30 ml) under argon atmosphere 50% NaH in oil (0.36 g, 7.5 mmol) was added in 3 portions during 30 minutes. The mixture was stirred at room temperature for one hour. Ethyl acetate was added and the crude product was washed twice with 0.5 M aqueous citric acid, aqueous sodium hydrogencarbonate, and water. The organic solvents dried over Na₂SO₄, filtrated and removed in vacuum. The residue was purified by flash chromatography on silica using ethyl acetate/heptane (1:4) as eluent to give the title compound (2.0 mg, 86%).

¹H NMR (400 MHz, DMSO-d₆): δ 8.79 (1H, d); 8.24 (1H, dd); 6.57 (1H, brs); 5.86 (1H, qv); 4.72 (1H, brt); 3.55 (1H, brs); 3.45 (1H, t); 2.99 (1H, m); 2.86 (2H, m); 2.76 (1H, qv); 2.58 (2H, q); 2.16 (1H, m); 1.90 (2H, d); 1.50 (2H, brt); 1.42 (11H, brs).

APCI-MS m/z: 365 [MH⁺−tBOC].

LC (method A) rt=14.5 min

### Preparation 8

### 3-(cis-4-aminocyclohexyl)-6-fluoro-1-(tetrahydro-3-thienyl)pyrido[2,3-d]pyrimidine-2,4(1H,3H)-dione Hydrochloride

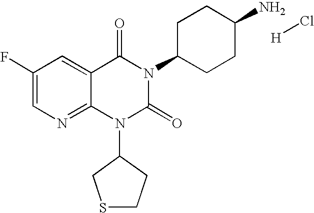

A mixture of tert-butyl {cis-4-[6-fluoro-2,4-dioxo-1-(tetrahydro-3-thienyl)-1,4-dihydropyrido[2,3-d]pyrimidin-3(2H)-yl]cyclohexyl}carbamate (0.7 g, 1.5 mmol) and 4 M HCl in 1,4-Dioxane (20 ml) was stirred at room temperature for two hours. The solvents were removed and the pure crude product was used directly.

APCI-MS m/z: 365 [MH⁺].

### EXAMPLE 1

### N-{cis-4-[6-Fluoro-2,4-dioxo-1-(tetrahydro-2H-thiopyran-4-yl)-1,4-dihydropyrido[2,3-d]pyrimidin-3(2H)-yl]cyclohexyl}cyclopropanecarboxamide

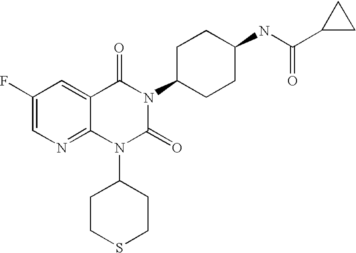

A mixture of 3-(cis-4-aminocyclohexyl)-6-fluoro-1-(tetrahydro-2H-thiopyran-4-yl)pyrido[2,3-d]pyrimidine-2,4(1H,3H)-dione hydrochloride (310 mg, 0.75 mmol), cyclopropanecarboxylic acid (100 mg, 1.16 mmol), HATU (380 mg, 1.0 mmol), HOAT (140 mg, 1.0 mmol) and DIEA (600 μl, 3.5 mmol) in NMP (20 ml) was stirred for 10 min at room temperature (at pH 8-9). Ethyl acetate was added and the crude product was washed twice with aqueous sodium hydrogencarbonate, 0.5 M aqueous citric acid and water. The organic solvents dried over Na₂SO₄, filtrated and removed in vacuum. The residue was purified by flash chromatography on silica using ethyl acetate/heptane (1:1) as eluent to give the title compound (280 mg, 84%).

¹H NMR (400 MHz, DMSO-d₆): δ 8.78 (1H, d); 8.23 (1H, dd); 7.93 (1H, brd); 5.21 (1H, brs); 4.74 (1H, t); 3.79 (1H, brs); 2.86-2.69 (6H, m); 2.63 (2H, q); 2.01-1.86 (4H, m); 1.81 (1H, m); 1.53 (2H, t); 1.44 (2H, brd); 0.66 (4H, m).

APCI-MS m/z: 447 [MH⁺].

### EXAMPLE 2

### N-{cis-4-[6-Fluoro-2,4-dioxo-1-(tetrahydro-2H-thiopyran-4-yl)-1,4-dihydropyrido[2,3-d]pyrimidin-3(2H)-yl]cyclohexyl}imidazo[1,2-a]pyridine-2-carboxamide

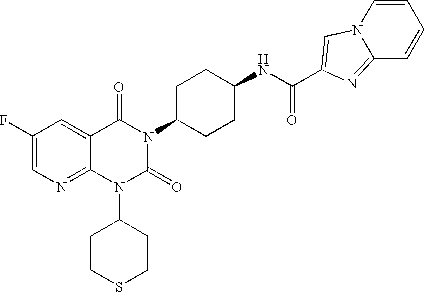

A mixture of 3-(cis-4-aminocyclohexyl)-6-fluoro-1-(tetrahydro-2H-thiopyran-4-yl)pyrido[2,3-d]pyrimidine-2,4(1H,3H)-dione hydrochloride (600 mg, 1.5 mmol), imidazo[1,2-a]pyridine-2-carboxylic acid (292 mg, 1.8 mmol), HATU (685 mg, 1.8 mmol), and DIEA (1.02 ml, 6 mmol) in NMP (80 ml) was stirred for 1 h at room temperature (at pH 8-9). Ethyl acetate was added and the crude product was washed twice with aqueous sodium hydrogencarbonate, 0.5 M aqueous citric acid and water. The organic solvents dried over Na₂SO₄, filtrated and removed in vacuum. The residue was recrystallised in ethyl acetate (5 ml) to give the title compound (750 mg, 96%).

¹H NMR (400 MHz, DMSO-d₆): δ 8.79 (1H, d); 8.68 (1H, d); 8.52 (1H, s); 8.25 (1H, dd); 7.94 (1H, brs); 7.75 (1H, d); 7.49 (1H, t); 7.10 (1H, t); 5.22 (1H, brs); 4.84 (1H, t); 4.16 (1H, s); 2.84-2.69 (6H, m); 2.61 (2H, q); 1.99 (4H, m); 1.71 (2H, t); 1.57 (2H, brd)

APCI-MS m/z: 523 [MH⁺].

### EXAMPLES 3-31

The following compounds were synthesised following the procedure described in Example 1.

| Ex | Compound | ¹H NMR | m/z |
|---|---|---|---|
| 3 | N-{cis-4-[6-fluoro-2,4-dioxo- | (400 MHz, DMSO-d₆): δ 9.47 (1H, s); | 534.3 |
| | 1-(tetrahydro-2H-thiopyran-4- | 8.76 (1H, d); 8.64 (1H, d); 8.59 (1H, d); | |
| | yl)-1,4-dihydropyrido[2,3- | 8.28 (1H, dd); 8.20 (1H, d); | |
| | d]pyrimidin-3(2H)- | 7.76-7.73 (2H, m); 5.18 (1H, brs); 4.87 (1H, t); | |
| | yl]cyclohexyl}quinoline-8- | 4.45 (1H, s); 2.85-2.72 (8H, m); | |
| | carboxamide | 1.98 (2H, d); 1.85 (2H, d); 1.72 (2H, t); | |
| | | 1.60 (2H, d). | |
| 4 | N-{cis-4-[6-fluoro-2,4-dioxo- | (500 MHz, DMSO-d₆): δ 8.75 (1H, d); | 523.3 |
| | 1-(tetrahydro-2H-thiopyran-4- | 8.71 (1H, d); 8.19 (1H, dd); 7.76 (1H, | |
| | yl)-1,4-dihydropyrido[2,3- | d); 7.68-7.65 (1H, m); 7.29-7.26 (1H, | |
| | d]pyrimidin-3(2H)- | m); 7.03-7.00 (2H, m); 5.16 (1H, brs); | |
| | yl]cyclohexyl}pyrazolo[1,5- | 4.79 (1H, t); 4.12 (1H, s); | |
| | a]pyridine-2-carboxamide | 2.77-2.66 (5H, m); *(3H in solvent peak) | |
| | | 1.98-1.91 (4H, m); 1.64 (2H, t); 1.51 (2H, d). | |
| 5 | N-{cis-4-[6-fluoro-2,4-dioxo- | (500 MHz, DMSO-d₆): δ 8.74 (1H, d); | 523.3 |
| | 1-(tetrahydro-2H-thiopyran-4- | 8.22 (1H, dd); 8.16 (1H, d); | |
| | yl)-1,4-dihydropyrido[2,3- | 7.62-7.59 (2H, m); 7.40 (1H, t); 7.24 (1H, t); | |
| | d]pyrimidin-3(2H)- | 5.18 (1H, brs); 4.81 (1H, t); 4.19 (1H, d); | |
| | yl]cyclohexyl}-1H-indazole-3- | 2.79-2.67 (5H, m); *(3H in solvent | |
| | carboxamide | peak) 1.99-1.93 (4H, m); 1.67 (2H, t); | |
| | | 1.53 (2H, d). | |
| 6 | N-[2-({cis-4-[6-fluoro-2,4- | (500 MHz, DMSO-d₆): δ 8.99 (1H, t); | 556.3 |
| | dioxo-1-(tetrahydro-2H- | 8.74 (1H, t); 8.13 (1H, dd); 7.90 (1H, | |
| | thiopyran-4-yl)-1,4- | d); 7.86 (1H, d); 7.36 (1H, t); | |
| | dihydropyrido[2,3- | 6.89-6.86 (2H, m); 5.18 (1H, brs); 4.70 (1H, | |
| | d]pyrimidin-3(2H)- | t); 4.00 (2H, d); 3.84 (1H, s); | |
| | yl]cyclohexyl}amino)-2- | 2.80-2.67 (4H, m); *(4H in solvent peak) | |
| | oxoethyl]-2-hydroxybenzamide | 1.92 (2H, d); 1.85 (2H, d); 1.53 (2H, t); | |
| | | 1.42 (2H, d). | |
| 7 | N-{cis-4-[6-fluoro-2,4-dioxo- | (500 MHz, DMSO-d₆): δ 8.74 (1H, d); | 529.3 |
| | 1-(tetrahydro-2H-thiopyran-4- | 8.21 (1H, dd); 7.59 (1H, d); 7.41 (1H, | |
| | yl)-1,4-dihydropyrido[2,3- | d); 7.31 (1H, d); 6.99 (1H, d); 5.19 (1H, | |
| | d]pyrimidin-3(2H)- | brs); 4.78 (1H, t); 3.93 (1H, s); | |
| | yl]cyclohexyl}-3-hydroxy-4- | 3.80 (3H, s); 2.81-2.67 (6H, m); *(2H in | |
| | methoxybenzamide | solvent peak) 2.02 (2H, d); | |
| | | 1.95-1.93 (2H, m); 1.54 (2H, t); 1.43 (2H, d). | |
| 8 | N-{cis-4-[6-fluoro-2,4-dioxo- | (500 MHz, DMSO-d₆): δ 8.74 (1H, d); | 513.3 |
| | 1-(tetrahydro-2H-thiopyran-4- | 8.19 (1H, dd); 7.88 (1H, d); 7.05 (1H, | |
| | yl)-1,4-dihydropyrido[2,3- | t); 6.72 (1H, d); 6.67 (1H, s); 6.56 (1H, | |
| | d]pyrimidin-3(2H)- | dd); 5.18 (1H, brs); 4.69 (1H, t); | |
| | yl]cyclohexyl}-2-(3- | 3.75 (1H, s); 2.78-2.67 (6H, m); *(2H in | |
| | hydroxyphenyl)acetamide | solvent peak) 1.93 (2H, dd); 1.83 (2H, | |
| | | d); 1.50 (2H, t); 1.40 (2H, d). | |
| 9 | N-{cis-4-[6-fluoro-2,4-dioxo- | (500 MHz, DMSO-d₆): δ 11.91 (1H, s); | 513.3 |
| | 1-(tetrahydro-2H-thiopyran-4- | 8.74 (1H, d); 8.41 (1H, d); 8.18 (1H, | |
| | yl)-1,4-dihydropyrido[2,3- | dd); 7.86 (1H, d); 6.74-6.73 (2H, m); | |
| | d]pyrimidin-3(2H)- | 5.18 (1H, brs); 4.77 (1H, s); 4.08 (1H, | |
| | yl]cyclohexyl}-2-hydroxy-4- | s); 2.78-2.66 (5H, m); *(3H in solvent | |
| | methylbenzamide | peak) 2.25 (3H, s); 1.98-1.91 (4H, m); | |
| | | 1.62 (2H, t); 1.49 (2H, d). | |
| 10 | N-{cis-4-[6-fluoro-2,4-dioxo- | (500 MHz, DMSO-d₆): δ 8.74 (1H, d); | 513.4 |
| | 1-(tetrahydro-2H-thiopyran-4- | 8.20 (1H, dd); 7.63 (1H, d); 7.29 (1H, | |
| | yl)-1,4-dihydropyrido[2,3- | d); 7.23 (1H, s); 7.15 (1H, d); 5.18 (1H, | |
| | d]pyrimidin-3(2H)- | brs); 4.77 (1H, dd); 3.94 (1H, s); | |
| | yl]cyclohexyl}-3-hydroxy-4- | 2.76-2.66 (5H, m); *(3H in solvent peak) | |
| | methylbenzamide | 2.13 (3H, s); 2.02 (2H, d); | |
| | | 1.94-1.92 (2H, m); 1.55 (2H, t); 1.43 (2H, d). | |
| 11 | N-{cis-4-[6-fluoro-2,4-dioxo- | (500 MHz, DMSO-d₆): δ 8.74 (1H, d); | 529.3 |
| | 1-(tetrahydro-2H-thiopyran-4- | 8.18 (1H, dd); 7.58 (1H, d); | |
| | yl)-1,4-dihydropyrido[2,3- | 7.42-7.41 (1H, m); 7.38 (1H, dd); 6.82 (1H, d); | |
| | d]pyrimidin-3(2H)- | 5.18 (1H, brs); 4.78 (1H, t); 3.92 (1H, | |
| | yl]cyclohexyl}-4-hydroxy-3- | s); 3.86 (3H, s); 2.75-2.67 (6H, m); | |
| | methoxybenzamide | *(2H in solvent peak) 2.05 (2H, d); | |
| | | 1.94-1.92 (2H, m); 1.54 (2H, t); 1.43 (2H, | |
| | | d). | |
| 12 | (2R)-2-cyclohexyl-N-{cis-4- | (500 MHz, DMSO-d₆): δ 8.73 (1H, d); | 519.4 |
| | [6-fluoro-2,4-dioxo-1- | 8.19 (1H, dd); 7.37 (1H, d); 5.16 (1H, | |
| | (tetrahydro-2H-thiopyran-4- | brs); 4.73 (1H, t); 3.91 (1H, d); | |
| | yl)-1,4-dihydropyrido[2,3- | 3.70 (1H, d); 2.75-2.66 (4H, m); *(4H in | |
| | d]pyrimidin-3(2H)- | solvent peak) 1.93-1.91 (2H, m); | |
| | yl]cyclohexyl}-2- | 1.75 (2H, t); 1.67-1.53 (8H, m); 1.46 (2H, | |
| | hydroxyacetamide | d); 1.40-1.38 (1H, m); 1.24-1.02 (6H, | |
| | | m). | |
| 13 | methyl 5-({cis-4-[6-fluoro-2,4- | (500 MHz, DMSO-d₆): δ 8.73 (1H, d); | 507.3 |
| | dioxo-1-(tetrahydro-2H- | 8.17 (1H, dd); 7.66 (1H, d); 5.18 (1H, | |
| | thiopyran-4-yl)-1,4- | brs); 4.68 (1H, t); 3.75 (1H, s); *(4H in | |
| | dihydropyrido[2,3- | solvent peak) 2.75-2.67 (4H, m); *(3H | |
| | d]pyrimidin-3(2H)- | in solvent peak) 2.29 (2H, t); 2.17 (2H, | |
| | yl]cyclohexyl}amino)-5- | t); 1.93-1.91 (2H, m); 1.83 (2H, d); | |
| | oxopentanoate | 1.73 (2H, quintet); 1.48 (2H, t); | |
| | | 1.38 (2H, d). | |
| 14 | N-{cis-4-[6-fluoro-2,4-dioxo- | (500 MHz, DMSO-d₆): δ 9.63 (1H, s); | 499.3 |
| | 1-(tetrahydro-2H-thiopyran-4- | 8.73 (1H, d); 8.20 (1H, dd); 7.77 (1H, | |
| | yl)-1,4-dihydropyrido[2,3- | d); 7.32 (1H, d); 7.28-7.23 (2H, m); | |
| | d]pyrimidin-3(2H)- | 6.89 (1H, dd); 5.16 (1H, brs); 4.76 (1H, | |
| | yl]cyclohexyl}-3- | t); 3.93 (1H, s); 2.76-2.66 (5H, m); | |
| | hydroxybenzamide | *(3H in solvent peak) 2.04 (2H, d); | |
| | | 1.94-1.92 (2H, m); 1.55 (2H, t); 1.43 (2H, | |
| | | d). | |
| 15 | N-{cis-4-[6-fluoro-2,4-dioxo- | (500 MHz, DMSO-d₆): δ 8.74 (1H, d); | 513.3 |
| | 1-(tetrahydro-2H-thiopyran-4- | 8.19 (1H, dd); 7.78 (1H, d); 7.09 (2H, | |
| | yl)-1,4-dihydropyrido[2,3- | d); 6.65 (2H, d); 5.19 (1H, brs); | |
| | d]pyrimidin-3(2H)- | 4.69 (1H, t); 3.72 (1H, s); *(2H in solvent | |
| | yl]cyclohexyl}-2-(4- | peak) 2.77-2.68 (5H, m); *(3H in | |
| | hydroxyphenyl)acetamide | solvent peak) 1.95-1.92 (2H, m); | |
| | | 1.82 (2H, d); 1.47 (2H, t); 1.38 (2H, d). | |
| 16 | N-{cis-4-[6-fluoro-2,4-dioxo- | (500 MHz, DMSO-d₆): δ 8.73 (1H, d); | 499.3 |
| | 1-(tetrahydro-2H-thiopyran-4- | 8.20 (1H, dd); 7.75 (2H, d); 7.58 (1H, | |
| | yl)-1,4-dihydropyrido[2,3- | d); 6.81 (2H, d); 5.18 (1H, brs); | |
| | d]pyrimidin-3(2H)- | 4.78 (1H, t); 3.93 (1H, s); 2.76-2.65 (5H, | |
| | yl]cyclohexyl}-4- | m); *(3H in solvent peak) 2.02 (2H, d); | |
| | hydroxybenzamide | 1.94-1.92 (2H, m); 1.54 (2H, t); | |
| | | 1.43 (2H, d). | |
| 17 | N-{cis-4-[6-fluoro-2,4-dioxo- | (500 MHz, DMSO-d₆): δ 11.88 (1H, s); | 499.2 |
| | 1-(tetrahydro-2H-thiopyran-4- | 8.73 (1H, d); 8.53 (1H, d); 8.18 (1H, | |
| | yl)-1,4-dihydropyrido[2,3- | dd); 7.96 (1H, d); 7.36 (1H, dd); | |
| | d]pyrimidin-3(2H)- | 6.94-6.90 (2H, m); 5.15 (1H, brs); 4.77 (1H, | |
| | yl]cyclohexyl}-2- | t); 4.10 (1H, s); 2.76-2.67 (5H, m); | |
| | hydroxybenzamide | *(3H in solvent peak) 1.98-1.91 (4H, | |
| | | m); 1.63 (2H, t); 1.50 (2H, d). | |
| 18 | N-{cis-4-[6-fluoro-2,4-dioxo- | (500 MHz, DMSO-d₆): δ 8.73 (1H, d); | 489.4 |
| | 1-(tetrahydro-2H-thiopyran-4- | 8.18 (1H, dd); 7.46 (1H, d); 5.18 (1H, | |
| | yl)-1,4-dihydropyrido[2,3- | brs); 4.68 (1H, t); 3.71 (1H, s); | |
| | d]pyrimidin-3(2H)- | 2.75-2.67 (5H, m); 2.29-2.23 (1H, m); *(3H | |
| | yl]cyclohexyl}cyclohexanecarboxamide | in solvent peak) 1.93-1.91 (2H, m); | |
| | | 1.82 (2H, d); 1.70-1.65 (4H, m); | |
| | | 1.59 (1H, d); 1.46 (2H, t); 1.38-1.07 (7H, | |
| | | m). | |
| 19 | N-{cis-4-[6-fluoro-2,4-dioxo- | (500 MHz, DMSO-d₆): δ 8.73 (1H, d); | 527.4 |
| | 1-(tetrahydro-2H-thiopyran-4- | 8.17 (1H, dd); 7.67 (1H, d); 7.02 (1H, | |
| | yl)-1,4-dihydropyrido[2,3- | t); 6.62 (1H, d); 6.58 (1H, s); 6.52 (1H, | |
| | d]pyrimidin-3(2H)- | dd); 5.17 (1H, brs); 4.67 (1H, s); | |
| | yl]cyclohexyl}-3-(3- | 3.77 (1H, s); 2.74-2.67 (5H, m); *(7H in | |
| | hydroxyphenyl)propanamide | solvent peak) 1.93-1.91 (2H, m); | |
| | | 1.82 (2H, d); 1.48 (2H, t); 1.37 (2H, d). | |
| 20 | N-{cis-4-[6-fluoro-2,4-dioxo- | (500 MHz, DMSO-d₆): δ 8.73 (1H, d); | 500.4 |
| | 1-(tetrahydro-2H-thiopyran-4- | 8.50 (1H, s); 8.23-8.20 (3H, m); | |
| | yl)-1,4-dihydropyrido[2,3- | 7.54 (1H, dd); 7.42 (1H, dd); 5.18 (1H, brs); | |
| | d]pyrimidin-3(2H)- | 4.81 (2H, t); 4.15 (1H, s); | |
| | yl]cyclohexyl}-3- | 2.76-2.66 (5H, m); *(3H in solvent peak) | |
| | hydroxypyridine-2- | 1.97-1.92 (4H, m); 1.70 (2H, t); 1.55 (2H, d). | |
| | carboxamide | | |
| 21 | N-{cis-4-[6-fluoro-2,4-dioxo- | (500 MHz, DMSO-d₆): δ 8.72 (1H, d); | 463.3 |
| | 1-(tetrahydro-2H-thiopyran-4- | 8.17 (1H, dd); 7.60 (1H, d); 5.17 (1H, | |
| | yl)-1,4-dihydropyrido[2,3- | brs); 4.68 (1H, t); 3.76 (1H, s); | |
| | d]pyrimidin-3(2H)- | 2.74-2.66 (5H, m); *(3H in solvent peak) | |
| | yl]cyclohexyl}-3- | 2.01 (2H, d); 1.98-1.91 (3H, m); | |
| | methylbutanamide | 1.83 (2H, d); 1.48 (2H, t); 1.38 (2H, d); | |
| | | 0.87 (6H, d). | |
| 22 | N-{cis-4-[6-fluoro-2,4-dioxo- | (500 MHz, DMSO-d₆): δ 8.74 (1H, d); | 513.4 |
| | 1-(tetrahydro-2H-thiopyran-4- | 8.21 (1H, dd); 7.61 (1H, d); 7.44 (2H, | |
| | yl)-1,4-dihydropyrido[2,3- | d); 7.30 (2H, t); 7.23 (1H, t); 5.17 (1H, | |
| | d]pyrimidin-3(2H)- | brs); 4.95 (1H, s); 4.74 (1H, t); | |
| | yl]cyclohexyl}-2-hydroxy-2- | 3.86 (1H, s); 2.76-2.68 (5H, m); *(3H in | |
| | phenylacetamide | solvent peak) 1.93 (2H, s); 1.76 (2H, t); | |
| | | 1.59-1.43 (4H, m). | |
| 23 | N-{cis-4-[6-fluoro-2,4-dioxo- | (500 MHz, DMSO-d₆): δ 8.73 (1H, d); | 483.3 |
| | 1-(tetrahydro-2H-thiopyran-4- | 8.20 (1H, dd); 7.90-7.87 (3H, m); | |
| | yl)-1,4-dihydropyrido[2,3- | 7.53-7.46 (3H, m); 5.18 (1H, brs); 4.79 (1H, | |
| | d]pyrimidin-3(2H)- | t); 3.95 (1H, s); 2.76-2.65 (5H, m); | |
| | yl]cyclohexyl}benzamide | *(3H in solvent peak) 2.06 (2H, d); | |
| | | 1.94-1.92 (2H, m); 1.56 (2H, t); 1.44 (2H, | |
| | | d). | |
| 24 | N-{cis-4-[6-fluoro-2,4-dioxo- | (500 MHz, DMSO-d₆): δ 8.73 (1H, d); | 473.2 |
| | 1-(tetrahydro-2H-thiopyran-4- | 8.20 (1H, dd); 7.84 (1H, t); 7.57 (1H, | |
| | yl)-1,4-dihydropyrido[2,3- | d); 7.16 (1H, d); 6.62 (1H, dd); | |
| | d]pyrimidin-3(2H)- | 5.17 (1H, brs); 4.76 (1H, t); 3.97 (1H, s); | |
| | yl]cyclohexyl}-2-furamide | 2.76-2.67 (5H, m); *(3H in solvent | |
| | | peak) 1.99 (2H, d); 1.93-1.92 (2H, m); | |
| | | 1.58 (2H, t); 1.45 (2H, d). | |
| 25 | N-{cis-4-[6-fluoro-2,4-dioxo- | (500 MHz, DMSO-d₆): δ 8.85 (1H, d); | 523.3 |
| | 1-(tetrahydro-2H-thiopyran-4- | 8.73 (1H, d); 8.22-8.18 (3H, m); | |
| | yl)-1,4-dihydropyrido[2,3- | 7.90 (1H, s); 7.30 (1H, s); 5.18 (1H, brs); | |
| | d]pyrimidin-3(2H)- | 4.80 (1H, t); 4.17 (1H, s); | |
| | yl]cyclohexyl}imidazo[1,2- | 2.74-2.66 (5H, m); *(3H in solvent peak) | |
| | a]pyridine-8-carboxamide | 2.01 (2H, d); 1.94-1.92 (2H, m); 1.68 (2H, | |
| | | t); 1.52 (2H, d). | |
| 26 | N-{cis-4-[6-fluoro-2,4-dioxo- | (400 MHz, DMSO-d₆): δ 11.74 (1H, s); | 529 |
| | 1-(tetrahydro-2H-thiopyran-4- | 8.79 (1H, d); 8.60 (1H, d); 8.22 (1H, | |
| | yl)-1,4-dihydropyrido[2,3- | dd); 7.95 (1H, d); 7.34 (1H, dd); | |
| | d]pyrimidin-3(2H)- | 6.95 (1H, d); 5.20 (1H, brs); 5.11 (1H, brs); | |
| | yl]cyclohexyl}-2-hydroxy-5- | 4.82 (1H, t); 4.45 (2H, s); 4.15 (1H, | |
| | (hydroxymethyl)benzamide | brs); 2.84-2.69 (6H, m); 2.61 (2H, q); | |
| | | 1.99 (4H, m); 1.68 (2H, t); 1.55 (2H, d) | |
| 27 | N-{cis-4-[6-fluoro-2,4-dioxo- | (400 MHz, DMSO-d₆): δ 8.80 (1H, d); | 433 |
| | 1-(tetrahydro-3-thienyl)-1,4- | 8.25 (1H, dd); 5.87 (1H, m); 4.74 (1H, | |
| | dihydropyrido[2,3- | t); 3.78 (1H, brs); 3.46 (1H, t); *(1H in | |
| | d]pyrimidin-3(2H)- | solvent peak); 3.00 (1H, m); | |
| | yl]cyclohexyl}cyclopropanecarboxamide | 2.91-2.80 (2H, m); 2.77 (1H, m); 2.63 (2H, q); | |
| | | 2.17 (1H, m); 1.91 (2H, brd); 1.81 (1H, | |
| | | m); 1.53 (2H, t); 1.45 (2H, brd); | |
| | | 0.65 (4H, m) | |
| 28 | N-{cis-4-[6-fluoro-2,4-dioxo- | (400 MHz, DMSO-d₆): δ 8.80 (1H, d); | 509 |
| | 1-(tetrahydro-3-thienyl)-1,4- | 8.74 (1H, d); 8.59 (1H, brs); 8.27 (1H, | |
| | dihydropyrido[2,3- | dd); 8.05 (1H, brs); 7.75 (1H, d); | |
| | d]pyrimidin-3(2H)- | 7.56 (1H, t); 7.16 (1H, t); 5.86 (1H, m); | |
| | yl]cyclohexyl}imidazo[1,2- | 4.83 (1H, t); 4.15 (1H, brs); *(1H in solvent | |
| | a]pyridine-2-carboxamide | peak); 3.01 (1H, m); 2.91-2.83 (2H, m); | |
| | | 2.77 (1H, m); 2.63 (2H, q); 2.17 (1H, | |
| | | m); 2.03 (2H, brd); 1.71 (2H, m); | |
| | | 1.59 (2H, t) | |
| 29 | N-{cis-4-[6-fluoro-2,4-dioxo- | (400 MHz, DMSO-d₆): δ 8.80 (1H, d); | 515 |
| | 1-(tetrahydro-3-thienyl)-1,4- | 8.59 (1H, d); 8.24 (1H, dd); 7.94 (1H, | |
| | dihydropyrido[2,3- | d); 7.35 (1H, dd); 6.95 (1H, d); | |
| | d]pyrimidin-3(2H)- | 5.84 (1H, m); 4.82 (1H, t); 4.44 (2H, s); | |
| | yl]cyclohexyl}-2-hydroxy-5- | 4.16 (1H, brs); 3.47 (1H, t); *(2H in solvent | |
| | (hydroxymethyl)benzamide | peak); 3.00 (1H, m); 2.91-2.81 (2H, m); | |
| | | 2.77 (1H, m); 2.61 (2H, q); 2.17 (1H, | |
| | | m); 2.00 (2H, brd); 1.68 (2H, t); | |
| | | 1.57 (2H, brd) | |
| 30 | N-{cis-4-[6-fluoro-2,4-dioxo- | (400 MHz, DMSO-d₆): δ 11.90 (1H, s); | 485 |
| | 1-(tetrahydro-3-thienyl)-1,4- | δ 8.79 (1H, d); 8.59 (1H, d); 8.24 (1H, | |
| | dihydropyrido[2,3- | dd); 8.00 (1H, dd); 7.39 (1H, t); | |
| | d]pyrimidin-3(2H)- | 6.98 (1H, d); 6.94 (1H, t); 5.84 (1H, m); | |
| | yl]cyclohexyl}-2- | 4.83 (1H, t); 4.14 (1H, brs); 3.47 (1H, t); | |
| | hydroxybenzamide | 3.00 (1H, m); 2.91-2.81 (2H, m); | |
| | | 2.77 (1H, m); 2.63 (2H, q); 2.17 (1H, m); | |
| | | 2.01 (2H, brd); 1.67 (2H, t); 1.56 (2H, | |
| | | brd) | |
| 31 | N-{cis-4-[6-fluoro-2,4-dioxo- | (400 MHz, DMSO-d₆): δ 9.52 (1H, brs); | 499 |
| | 1-(tetrahydro-3-thienyl)-1,4- | δ 8.79 (1H, d); 8.27 (1H, dd); 7.68 (1H, | |
| | dihydropyrido[2,3- | d); 7.31 (1H, dd); 7.28 (1H, d); | |
| | d]pyrimidin-3(2H)- | 7.18 (1H, d); 5.86 (1H, m); 4.81 (1H, t); | |
| | yl]cyclohexyl}-2-hydroxy-4- | 3.97 (1H, brs); 3.46 (1H, t); 3.01 (1H, m); | |
| | methylbenzamide | 2.90-2.82 (2H, m); 2.76 (1H, m); | |
| | | 2.67 (2H, q); 2.18 (4H, brs); 2.08 (2H, brd); | |
| | | 1.59 (2H, t); 1.49 (2H, brd) | |

### EXAMPLE 32

### N-{cis-4-[2,4-dioxo-1-(tetrahydro-2H-thiopyran-4-yl)-1,4-dihydropyrido[2,3-d]pyrimidin-3(2H)-yl]cyclohexyl}imidazo[1,2-a]pyridine-2-carboxamide

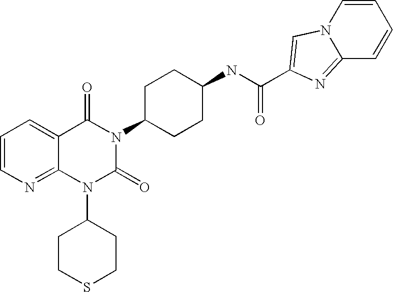

### Step (a) 2-(tetrahydro-2H-thiopyran-4-ylamino)nicotinic acid

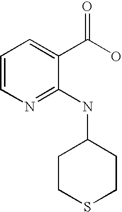

2-Chloronicotinic acid (5 g, 31.7 mmol) and potassium carbonate (5 g, 36.2 mmol) were added to dry NMP (20 ml) under nitrogen. Copper (80 mg, 1.26 mmol) and copper(1)bromide (200 mg, 1.39 mmol) were added, followed by 4-aminothiopyran (2 g, 17.1 mmol) and the mixture heated in an oil bath at 150° C. for 30 min. Mixture poured into water and the product that precipitated was collected by filtration, then triturated with acetonitrile to afford the sub-title compound (5 g, 66%).

¹H NMR (300 MHz, DMSO-d₆) δ 8.25 (dd, 1H), 8.13 (d, 1H), 8.08 (dd, 2H), 6.60 (m, 1H), 4.06 (m, 1H), 2.69 (m, 3H), 2.22 (d, 2H), 1.60 (m, 2H)

APCI (Multimode) m/z: 239 [M+H]

### Step (b) tert-butyl [cis-4′-({[2-(tetrahydro-2H-thiopyran-4-ylamino)pyridin-3-yl]carbonyl}amino)cyclohexyl]carbamate

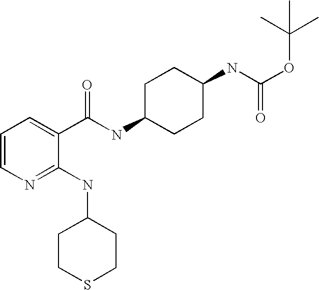

2-(Tetrahydro-thiopyran-4-ylamino)-nicotinic acid (3 g, 12.6 mmol) was dissolved in dry DMF (20 ml) and DIEA (4 ml, 22.9 mmol) was added, followed by HATU (4.79 g, 12.6 mmol) and the mixture allowed to stir at room temperature for 10 min. tert-Butyl (cis-4-aminocyclohexyl)carbamate (2.71 g, 12.6 mmol) was added and the mixture allowed to stir at room temperature overnight. Mixture poured into water and the product that precipitated was collected by filtration, then purified by flash chromatography on silica using ethyl acetate:isohexane (1:2) as the eluent to afford the sub-title compound (2.5 g, 45%).

¹H NMR (300 MHz, CDCl₃) δ 8.19 (d, 2H), 7.51 (d, 1H), 6.47 (dd, 1H), 5.95 (d, 1H), 4.54 (s, 1H), 4.07 (m, 3H), 3.67 (s, 1H), 2.73 (m, 4H), 2.32 (dd, 2H), 1.76 (m, 9H), 1.45 (s, 9H)

APCI (Multimode) m/z: 435 [M+H]

### Step (c) tert-butyl {cis-4-[2,4-dioxo-1-(tetrahydro-2H-thiopyran-4-yl)-1,4-dihydropyrido[2,3-d]pyrimidin-3(2H)-yl]cyclohexyl}carbamate

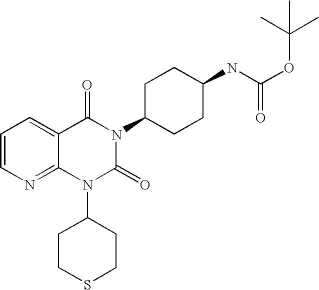

(4-{[2-(Tetrahydro-thiopyran-4-ylamino)-pyridine-3-carbonyl]-amino}-cyclohexyl)-carbamic acid tert-butyl ester (2.2 g, 5.1 mmol) was dissolved in dry NMP (5 ml) and carbonyldiimidazole (2.46 g, 15.2 mmol) was added, followed by 60% sodium hydride (0.61 g, 15.2 mmol) portion-wise over 5 min. The mixture was then heated to 70° C. for 15 min. Mixture allowed to cool to room temperature, then poured carefully into water. The crude product that precipitated was collected by filtration and purified by flash chromatography on silica using ethyl acetate:isohexane 1:3 as the eluent to afford the sub-title compound (1.8 g, 77%).

¹H NMR (400 MHz, CDCl₃) δ 8.63 (d, 1H), 8.43 (d, 1H), 7.19 (d, 1H), 5.06 (d, 1H), 4.91 (m, 1H), 3.94 (s, 1H), 2.92 (m, 2H), 2.76 (m, 2H), 2.57 (m, 2H), 2.02 (m, 2H), 1.91 (m, 2H), 1.62 (m, 7H), 1.48 (s, 9H)

APCI (ES+) m/z: 361 [MH+−BOC]

### Step (d) 3-(cis-4-aminocyclohexyl)-1-(tetrahydro-2H-thiopyran-4-yl)pyrido[2,3-d]pyrimidine-2,4(1H,3H)-dione

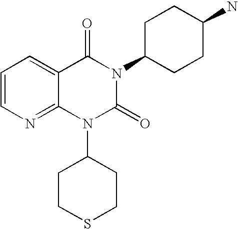

4.0M hydrogen chloride in dioxan (20 ml, 80 mmol) was added to {4-[2,4-Dioxo-1-(tetrahydro-thiopyran-4-yl)-1,4-dihydro-2H-pyrido[2,3-d]pyrimidin-3-yl]-cyclohexyl}-carbamic acid tert-butyl ester (230 mg, 0.5 mmol) and the mixture allowed to stir at room temperature for 2 h. The solvent was evaporated to dryness and the residue triturated with acetonitrile to afford the sub-title compound (200 mg, 100%).

¹H NMR (300 MHz, DMSO-d₆) δ 8.73 (m, 1H), 8.39 (m, 1H), 8.06 (s, 2H), 7.36 (dd, 1H), 5.31 (s, 1H), 4.76 (t, 1H), 3.43 (d, 1H), 2.79 (m, 5H), 2.58 (t, 2H), 1.96 (d, 4H), 1.77 (t, 2H), 1.57 (d, 3H).

APCI (Multimode) m/z: 361 [M+H]

### Step (e) N-{cis-4-[2,4-dioxo-1-(tetrahydro-2H-thiopyran-4-yl)-1,4-dihydropyrido[2,3-d]pyrimidin-3(2H)-yl]cyclohexyl}imidazo[1,2-a]pyridine-2-carboxamide

Imidazole[1,2a]pyridine-2-carboxylic acid (72 mg, 0.43 mmol) was dissolved in dry DMF (5 ml) and DIEA (0.2 ml, 1.15 mmol) was added, followed by HATU (167 mg, 0.43 mmol), and the mixture stirred for 10 min. 3-(4-amino-cyclohexyl)-1-(tetrahydro-thiopyran-4-yl)-1H-pyrido[2,3-d]pyrimidine-2,4-dione (220 mg, 0.43 mmol) was then added and the mixture stirred overnight. The mixture was poured into water and the solid that precipitated was collected by filtration, then purified by reverse phase HPLC (25-95% acetonitrile in aqueous ammonia) to afford the title compound (130 mg, 59%).

¹H NMR (400 MHz, DMSO-d₆) δ 8.73 (dd, 1H), 8.62 (d, 1H), 8.44 (s, 1H), 8.40 (dd, 1H), 7.81 (d, 1H), 7.73 (d, 1H), 7.40 (t, 1H), 7.35 (dd, 1H), 7.03 (t, 1H), 5.32 (s, 1H), 4.85 (t, 1H), 4.18 (s, 1H), 2.71 (m, 8H), 1.99 (d, 4H), 1.71 (t, 2H), 1.58 (d, 2H)

APCI (Multimode) m/z: 505 [M+H]

### EXAMPLE 33

### N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-5,6,7,8-tetrahydroimidazo[1,2-a]pyridine-2-carboxamide

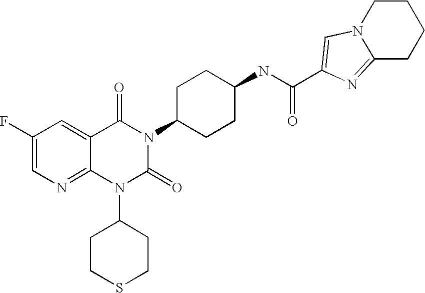

### Step (a) 5,6,7,8-tetrahydroimidazo[1,2-a]pyridine-2-carboxylic acid

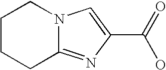

Imidazo[1,2-a]pyridine-2-carboxylic acid (500 mg, 3.1 mmol) was dissolved in ethanol (20 ml) and platinum oxide (100 mg) was added, and the mixture hydrogenated at 4 bar overnight. The mixture was filtered, and the filtrate evaporated to dryness to afford the sub-title compound (500 mg, 97%).

¹H NMR (400 MHz, DMSO-d₆) δ 7.63 (s, 1H), 3.96 (t, 2H), 2.70 (t, 2H), 1.85 (m, 4H)

### Step (b) N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-5,6,7,8-tetrahydroimidazo[1,2-a]pyridine-2-carboxamide

5,6,7,8-Tetrahydro-imidazo[1,2-a]pyridine-2-carboxylic acid (64 mg, 0.4 mmol) was dissolved in dry DMF (5 ml), and DIEA (0.2 ml, 1.15 mmol) was added, followed by HATU (160 mg, 0.42 mmol) and the mixture stirred for 10 min. 3-(4-amino-cyclohexyl)-1-(tetrahydro-thiopyran-4-yl)-1H-pyrido[2,3-d]pyrimidine-2,4-dione (150 mg, 0.4 mmol) was added and the mixture stirred overnight. The mixture was poured into water and the solid that precipitated was collected by filtration, then purified by reverse phase HPLC (25-95% acetonitrile in aqueous ammonia) to afford the title compound (124 mg, 59%).

¹H NMR (400 MHz, DMSO-d₆) δ 8.78 (d, 1H), 8.24 (dd, 1H), 7.51 (s, 1H), 7.35 (d, 1H), 5.29 (s, 1H), 4.80 (t, 1H), 4.09 (s, 1H), 3.99 (t, 2H), 2.76 (m, 6H), 2.57 (m, 1H), 2.50 (m, 5H), 1.97 (m, 2H), 1.91 (m, 4H), 1.66 (t, 2H), 1.54 (d, 2H),

APCI (Multimode) m/z: 527 [M+H]

### EXAMPLE 34

### N-{cis-4-[6-methyl-2,4-dioxo-1-(tetrahydro-2H-thiopyran-4-yl)-1,4-dihydropyrido[2,3-d]pyrimidin-3(2H)-yl]cyclohexyl}imidazo[1,2-a]pyridine-2-carboxamide

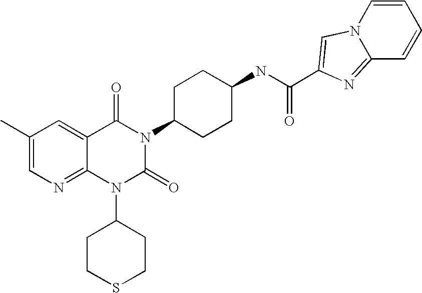

### Step (a) 2-chloro-5-methylnicotinic acid

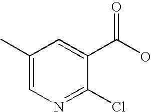

1.6 M solution of butyllithium in hexanes (6.03 g, 58.8 ml, 94.1 mmol) was added to THF (200 ml) and the mixture was cooled to −78° C. The mixture was treated dropwise with 2,2,6,6-tetramethylpiperidine (13.29 g, 17.6 ml, 94.1 mmol) and stirred at −78° C. for 30 min. The solution was then treated dropwise with 2-chloro-5-methyl-pyridine (12 g, 94.1 mmol) in THF (20 ml) and the reaction mixture was stirred at −78° C. for a further 2.5 h. The mixture was then poured onto dry ice and allowed to warm to room temperature overnight. The mixture was partitioned between diethyl ether and water, and the aqueous layer was then acidified to pH 2 with 2M HCl and the solvent concentrated to about 100 ml. The suspension was stored in the fridge for 48 hours, before being filtered and dried to afford the sub-title compound (5.6 g, 35%).

¹H NMR (400 MHz, DMSO-d₆) δ 13.70 (broad s, 1H), 8.38 (d, 1H), 8.05 (d, 1H), 2.34 (s, 3H)

### Step (b) 5-methyl-2-(tetrahydro-2H-thiopyran-4-ylamino)nicotinic acid

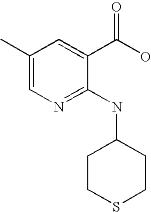

2-Chloro-5-methyl-nicotinic acid (2.5 g, 14.6 mmol) and K₂CO₃ (2.4 g, 17.5 mmol) were added to dry DMF (20 ml). Copper (55.6 mg, 0.87 mmol), copper(I)bromide (104.5 mg, 0.73 mmol) and tetrahydro-thiopyran-4-ylamine (2.9 g, 24.8 mmol) were added and the reaction mixture was stirred at 150° C. for 1.5 h. The mixture was cooled to room temperature and ethyl acetate was added. The reaction mixture was washed with 0.5M aq. citric acid. The aqueous layer was adjusted to pH 5 with aqueous ammonia and extracted with ethyl acetate (x3). The combined organic extracts were dried (anhydrous Na₂SO₄), filtered and evaporated. Water was added to the residue and the resulting solid was collected by filtration and dried in vacuo to afford the sub-title compound. (1.48 g, 40%).

¹H NMR (400 MHz, DMSO-d₆) δ 8.10 (dd, 1H), 7.94-7.88 (m, 1H), 4.06-3.98 (m, 1H), 2.75-2.61 (m, 4H), 2.21-2.18 (m, 2H), 2.14 (s, 3H), 1.57 (dtd, 2H)

### Step (c) tert-butyl [cis-4-({[5-methyl-2-(tetrahydro-2H-thiopyran-4-ylamino)pyridin-3-yl]carbonyl}amino)cyclohexyl]carbamate

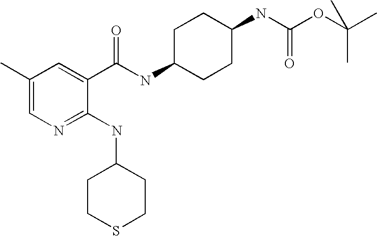

5-Methyl-2-(tetrahydro-thiopyran-4-ylamino)-nicotinic acid (1.48 g, 5.87 mmol) was dissolved in NMP (20 ml) and HATU (2.66 g, 7.04 mmol) was added, followed by DIEA (2.28 g, 3 ml, 17.6 mmol) and the mixture was stirred at room temperature for 10 min. (4-Amino-cyclohexyl)-carbamic acid tert-butyl ester (1.51 g, 7 mmol) was added and the reaction mixture was stirred at room temperature for 30 min. The solvents were evaporated in vacuo and the residue was stirred in water overnight. The resulting solid was collected by filtration, dissolved in ethyl acetate and dried (anhydrous magnesium sulfate), filtered and evaporated. The residue was purified by flash column chromatography on silica using 40% ethyl acetate in iso-hexane as eluent to afford the sub-title compound (704 mg, 27%).

¹H NMR (400 MHz, DMSO-d₆) δ 8.14 (d, 1H), 8.03 (d, 1H), 7.97 (d, 1H), 7.80 (d, 1H), 6.60 (s, 1H), 3.96-3.92 (m, 1H), 3.80-3.75 (m, 1H), 3.45-3.39 (m, 1H), 2.72-2.62 (m, 4H), 2.21-2.17 (m, 2H), 2.15 (s, 3H), 1.75-1.68 (m, 4H), 1.57-1.49 (m, 6H), 1.39 (s, 9H)

APCI (Multimode) m/z: 449 [M+H]

### Step (d) tert-butyl {cis-4-[6-methyl-2,4-dioxo-1-(tetrahydro-2H-thiopyran-4-yl)-1,4-dihydropyrido[2,3-d]pyrimidin-3(2H)-yl]cyclohexyl}carbamate

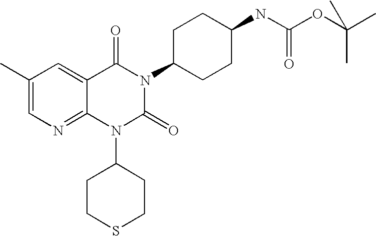

(4-{[5-Methyl-2-(tetrahydro-thiopyran-4-ylamino)-pyridine-3-carbonyl]-amino}-cyclohexyl)-carbamic acid tert-butyl ester (660 mg, 1.47 mmol) was dissolved in NMP (5 ml) and 1,1′-carbonyldiimidazole (716 mg, 4.41 mmol) was added, followed by portionwise addition of 60% sodium hydride in mineral oil (177 mg, 4.41 mmol). The reaction mixture was stirred at room temperature for 20 min before being heated at 70° C. for 2 h. Ice was then added to the reaction mixture and the mixture allowed to stir for a further 2 h. The resulting solid was collected by filtration, dissolved in ethyl acetate, dried (anhydrous magnesium sulphate), filtered and evaporated. The residue was purified by flash column chromatography on silica using 25% ethyl acetate in iso-hexane as eluent to afford the sub-title compound (268 mg, 38%).

¹H NMR (400 MHz, DMSO-d₆) δ 8.56 (d, 1H), 8.19 (d, 1H), 6.57 (s, 1H), 4.73 (t, 1H), 3.56 (s, 1H), 2.82-2.67 (m, 4H), 2.68-2.58 (m, 2H), 2.48-2.43 (m, 2H), 2.36 (s, 3H), 1.96-1.90 (m, 4H), 1.49 (t, 2H), 1.42 (s, 9H), 1.39-1.35 (m, 2H)

APCI (Multimode) m/z: 475 [M+H]

### Step (e) 3-(cis-4-aminocyclohexyl)-6-methyl-1-(tetrahydro-2H-thiopyran-4-yl)pyrido[2,3-d]pyrimidine-2,4(1H,3H)-dione

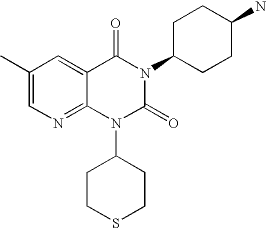

4.0M hydrogen chloride in 1,4-dioxane (10 ml, 40 mmol) was added to {4-[6-Methyl-2,4-dioxo-1-(tetrahydro-thiopyran-4-yl)-1,4-dihydro-2H-pyrido[2,3-d]pyrimidin-3-yl]-cyclohexyl}-carbamic acid tert-butyl ester (190 mg, 0.4 mmol) and the solution was stirred at room temperature for 1 h. The solvent was evaporated and the residue was partitioned between chloroform and saturated sodium carbonate solution. The aqueous was extracted a further 3 times with chloroform. The combined organic extracts were dried (anhydrous Na₂SO₄), filtered and evaporated to afford the sub-title compound (137 mg, 92%).

¹H NMR (400 MHz, DMSO-d₆) δ 8.56 (d, 1H), 8.20 (d, 1H), 4.76-4.69 (m, 1H), 3.07 (s, 1H), 2.83-2.68 (m, 9H), 2.38 (s, 3H), 1.97-1.88 (m, 2H), 1.62 (d, 2H), 1.55 (d, 1H), 1.50-1.39 (m, 2H), 1.33 (d, 2H), 1.23 (s, 1H)

### Step (f) N-{cis-4-[6-methyl-2,4-dioxo-1-(tetrahydro-2H-thiopyran-4-yl)-1,4-dihydropyrido[2,3-d]pyrimidin-3(2H)-yl]cyclohexyl}imidazo[1,2-a]pyridine-2-carboxamide

Imidazo[1,2-a]pyridine-2-carboxylic acid (71.2 mg, 0.44 mmol) was suspended in DMF (2 ml) and DIEA (142 mg, 191 ul, 1.1 mmol) was added followed by HATU (167 mg, 0.44 mmol) and the reaction mixture was stirred at room temperature for 10 min. A suspension of 3-(4-Amino-cyclohexyl)-6-methyl-1-(tetrahydro-thiopyran-4-yl)-1H-pyrido[2,3-d]pyrimidine-2,4-dione (137 mg, 0.37 mmol) in DMF (2 ml) was added and the reaction mixture was stirred at room temperature for 48 hours. The solvent was evaporated, and the residue was stirred in water. The resulting solid was collected by filtration and purified by reverse phase HPLC (25-95% acetonitrile in aqueoues ammonia) to afford the title compound (100 mg, 53%).

¹H NMR (300 MHz, DMSO-d₆) δ 8.65-8.55 (m, 2H), 8.43 (s, 1H), 8.24 (s, 1H), 7.84-7.67 (m, 2H), 7.40 (t, 1H), 7.02 (t, 1H), 5.26 (s, 1H), 4.85 (d, 1H), 4.20 (s, 1H), 2.89-2.70 (m, 6H), 2.63 (d, 2H), 2.39 (s, 3H), 2.03-1.91 (m, 4H), 1.72 (t, 2H), 1.58 (d, 2H)

APCI (Multimode) m/z: 519 [M+H]

### EXAMPLE 35 and 36

### a) N-{cis-4-[6-Fluoro-1-[(1r)-1-oxidotetrahydro-2H-thiopyran-4-yl]-2,4-dioxo-1,4-dihydropyrido[2,3-d]pyrimidin-3(2H)-yl]cyclohexyl}cyclopropanecarboxamide and

### b) N-{cis-4-[6-Fluoro-1-[(1s)-1-oxidotetrahydro-2H-thiopyran-4-yl]-2,4-dioxo-1,4-dihydropyrido[2,3-d]pyrimidin-3(2H)-yl]cyclohexyl}cyclopropanecarboxamide

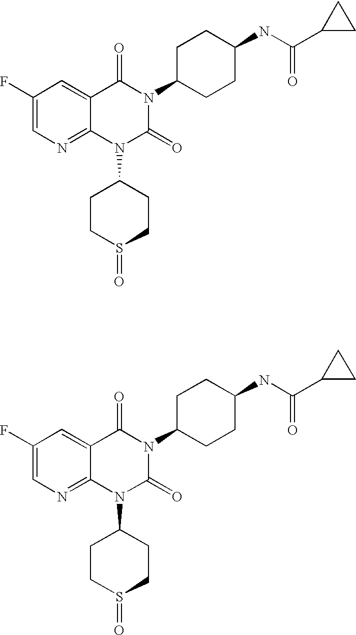

To solution of N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}imidazo[1,2-a]pyridine-2-carboxamide (90 mg, 0.2 mmol) in THF (20 ml) at −10° C., m-chloroperbenzoic acid (70-75%) (50 mg, 0.2 mmol) was added in three portions. Stirring was continued at −10° C. for 30 minutes and aqueous Na₂SO₃ (5%, 5 ml) was added and extracted with ethyl acetate. The organic solvents washed with aqueous sodium hydrogencarbonate, brine, water and dried over Na₂SO₄, filtered and evaporated. Purification on preparative HPLC gave a mixture of the titled compounds (90 mg, 97%) in a ratio a/b 5:1. The two isomers were isolated on preparative HPLC.

a) ¹H NMR (DMSO-d₆): δ 8.79 (1H, d), 8.25 (1H, dd), 7.96 (1H, brd), 5.45 (1H, brs), 4.72 (1H, t), 3.78 (1H, brs), 3.41 (2H, brs), 2.84 (4H, m), 2.62 (2H, q), 2.01-1.86 (4H, m), 1.81 (1H, m), 1.52 (2H, t), 1.43 (2H, brd), 0.65 (4H, m).

LC (method A) rt=6.1 min

b) ¹H NMR (DMSO-d₆): δ 8.77 (1H, d), 8.25 (1H, dd), 7.99 (1H, brd), 5.42 (1H, brs), 4.75 (1H, t), 3.78 (1H, brs), * (2H in solvent peak), 3.00 (2H, brd), 2.84 (2H, t), 2.64 (2H, m), 1.90 (2H, brd), 1.83 (1H, m), 1.68 (2H, d), 1.53 (2H, brt), 1.45 (2H, brd), 0.65 (4H, m).

LC (method A) rt=6.3 min

APCI-MS m/z: 463 [MH+].

### EXAMPLE 37

### N-{cis-4-[6-fluoro-1-(1-oxidotetrahydro-2H-thiopyran-4-yl)-2,4-dioxo-1,4-dihydropyrido[2,3-d]pyrimidin-3(2H)-yl]cyclohexyl}imidazo[1,2-a]pyridine-2-carboxamide

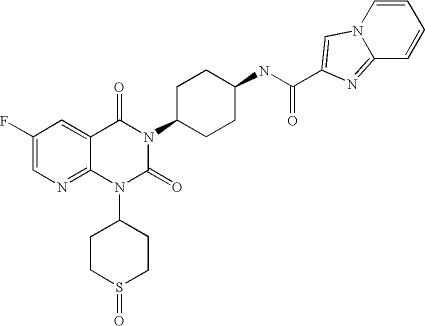

This title compound was made in a similar manner to Example 35.

¹H NMR (400 MHz, DMSO-d₆): δ 8.78 (1H, d), 8.60 (1H, d), 8.41 (1H, s), 8.27 (1H, dd), 7.75 (1H, d), 7.70 (1H, d), 7.39 (1H, t), 7.01 (1H, t), 5.45 (1H, brs), 4.82 (1H, t), 4.17 (1H, s), 3.40 (2H, brs), 2.85 (4H, brm), 2.59 (2H, q), 1.98 (4H, brm), 1.70 (2H, t), 1.57 (2H, t).

APCI-MS m/z: 539 [MH⁺].

LC (method A) rt=5.5 min

### EXAMPLE 38

### N-{cis-4-[1-(1,1-Dioxidotetrahydro-2H-thiopyran-4-yl)-6-fluoro-2,4-dioxo-1,4-dihydropyrido[2,3-d]pyrimidin-3(2H)-yl]cyclohexyl}cyclopropanecarboxamide

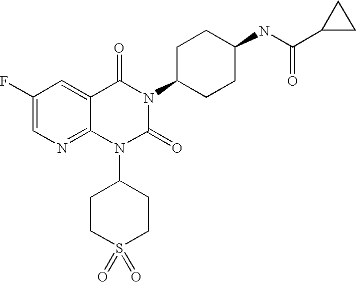

To solution of N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}imidazo[1,2-a]pyridine-2-carboxamide (90 mg, 0.2 mmol) in THF (20 ml) at 0° C., m-chloroperbenzoic acid (70-75%) (120 mg, 0.48 mmol) was added in three portions. Stirring was continued at 0° C. for 2 hours and aqueous Na₂SO₃ (5%, 10 ml) was added and extracted with ethyl acetate. The organic solvents washed with aqueous sodium hydrogencarbonate, brine, water and dried over Na₂SO₄, filtered and evaporated. The residue was purified by flash chromatography on silica using ethyl acetate/heptane (4:1) as eluent to give the title compound (95 mg, 99

¹H NMR (DMSO-d₆): δ 8.78 (1H, d), 8.25 (1H, dd), 7.95 (1H, brd), 5.58 (1H, brs), 4.74 (1H, t), 3.79 (1H, brs), 3.43 (2H, t), 3.24-3.07 (4H, m), 2.64 (2H, m), 2.01 (2H, brd), 1.90 (2H, brd), 1.82 (1H, m), 1.53 (2H, t), 1.45 (2H, brd), 0.65 (4H, m).

APCI-MS m/z: 479 [MH⁺].

### EXAMPLE 39

### N-{cis-4-[1-(1,1-Dioxidotetrahydro-2H-thiopyran-4-yl)-6-fluoro-2,4-dioxo-1,4-dihydropyrido[2,3-d]pyrimidin-3(2H)-yl]cyclohexyl}-2-hydroxy-5-(hydroxymethyl)benzamide

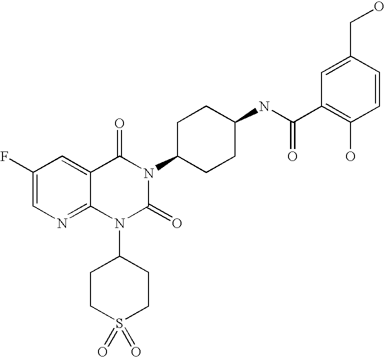

This title compound was made in a similar manner to Example 38.

¹H NMR (400 MHz, DMSO-d₆): δ 11.78 (1H, s), 8.78 (1H, d), 8.59 (1H, d), 8.23 (1H, dd), 7.95 (1H, d), 7.34 (1H, dd), 6.94 (1H, d), 5.55 (1H, brs), 5.12 (1H, brs), 4.81 (1H, t), 4.45 (2H, s), 4.14 (1H, brs), 3.43 (2H, t), 3.23-3.08 (4H, m), 2.61 (2H, q), 2.00 (4H, m), 1.68 (2H, t), 1.56 (2H, d).

APCI-MS m/z: 561 [MH⁺].

### EXAMPLE 40

### N-{cis-4-[1-(1,1-Dioxidotetrahydro-2H-thiopyran-4-yl)-6-fluoro-2,4-dioxo-1,4-dihydropyrido[2,3-d]pyrimidin-3(2H)-yl]cyclohexyl}imidazo[1,2-a]pyridine-2-carboxamide

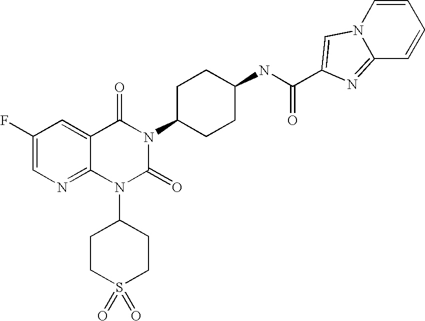

This title compound was made in a similar manner to Example 38.

¹H NMR (400 MHz, DMSO-d₆): δ 8.78 (1H, d), 8.68 (1H, d), 8.51 (1H, s), 8.27 (1H, dd), 7.93 (1H, brs), 7.74 (1H, d), 7.48 (1H, t), 7.09 (1H, t), 5.57 (1H, brs), 4.84 (1H, t), 4.17 (1H, s), * (2H in solvent peak), 3.21 (2H, brt), 3.11 (2H, brd), 2.62 (2H, q), 2.01 (4H, m), 1.72 (2H, t), 1.58 (2H, brd).

APCI-MS m/z: 555 [MH⁺].

### EXAMPLE 41

### 6-fluoro-N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}imidazo[1,2-a]pyridine-2-carboxamide

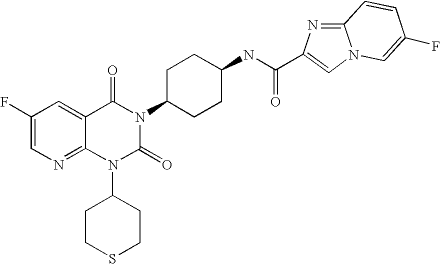

3-(4-Amino-cyclohexyl)-6-fluoro-1-(tetrahydro-thiopyran-4-yl)-1H-pyrido[2,3-d]pyrimidine-2,4-dione (189 mg, 0.5 mmol) and 6-fluoro-imidazo[1,2-a]pyridine-2-carboxylic acid ethyl ester (90 mg, 0.5 mmol) and DIEA (1 ml, 5.5 mmol) were dissolved in NMP (6 ml), and HATU (210 mg, 0.55 mmol) added. The reaction mixture was stirred for 40 min then poured into water and extracted with diethylether. The organic extract was evaporated and the residue purified by flash chromatography on silica using ethyl acetate:hexane (3:1) as eluent. The resultant product was triturated with diethyl ether, stirred under diethyl ether for one hour and the title compound collected and dried (130 mg, 48%).

¹H NMR (300 MHz, DMSO-d₆) δ 8.50 (d, 1H), 8.21-8.12 (m, 2H), 8.10 (td, 1H), 7.86 (d, 1H), 7.72 (dd, 1H), 7.21 (ddd, 1H), 5.34-5.15 (m, 1H), 5.01 (dd, 1H), 4.52-4.38 (m, 1H), 3.12-2.86 (m, 4H), 2.84-2.65 (m, 4H), 2.20-1.92 (m, 4H), 1.84-1.72 (m, 2H), 1.71-1.60 (m, 2H)

APCI (Multimode) m/z: 541 [M+H]

### EXAMPLE 64b

### N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-2-hydroxy-2-(4-hydroxyphenyl)acetamide

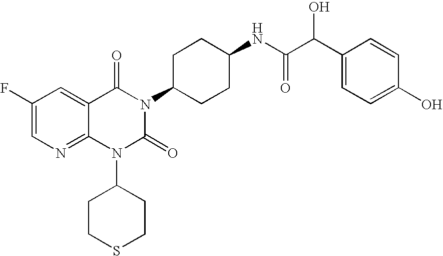

To a solution of 3-(4-Amino-cyclohexyl)-6-fluoro-1-(tetrahydro-thiopyran-4-yl)-1H-pyrido[2,3-d]pyrimidine-2,4-dione (0.25 g, 0.66 mmol) in NMP (2 ml) was added hydroxy(4-hydroxyphenyl)acetic acid (0.1223 g, 0.73 mmol) and DIPEA (0.588 mL, 3.31 mmol) followed by HATU (0.3016 g, 0.79 mmol). The reaction was stirred at room temperature for 12 hrs. Water was added and the title compound extracted with ethyl acetate. The organic phase was dried with anhydrous magnesium sulphate filtered and concentrated in vacuo to give a crude mixture. The residue was purified by reverse phase HPLC (32-40% acetonitrile in aqueoues ammonia) to afford the title compound (32 mg, 10%).

¹H NMR (400 MHz, DMSO-d₆): δ 9.31 (1H, s), 8.79 (1H, d), 8.26 (1H, dd), 7.57 (1H, d), 7.25 (2H, d), 6.71 (2H, dt), 6.14 (1H, d), 4.86 (1H, d), 4.79 (1H, s), 3.91 (1H, s), 2.82-2.73 (6H, m), 2.60-2.54 (2H, m), 1.99 (2H, s), 1.80 (2H, s), 1.65-1.50 (4H, m).

APCI (Multimode) m/z: 529 [+H]

The Examples in Tables 1, 2 and 3 were prepared in a similar manner to Example 1. The compounds were named using the IUPAC name program from Openeye (www.eyesopen.com) and stereochemical descriptors added by hand. (See www.eyesopen.com/products/applications/ogham.html). Where the compounds were isolated as primary or secondary amines the products were prepared by deprotection of a ‘BOC’ group using 4M HCl/Dioxane.

The compounds of Tables 1 and 2 are of formula (Ia) and the variable R1 in formula (Ia) has the meanings given in these Tables. Each of the listed groups of R1 in Tables 1 and 2 includes R1 and this indicates the point of attachment of the group to the formula (Ia).

TABLE 1

| | |
|---|---|
| | (Ia) |
| 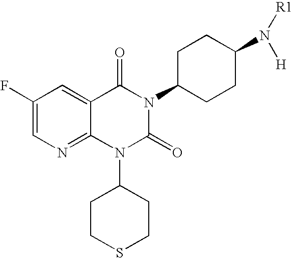 | |

| Example | | | | |
|---|---|---|---|---|
| Number | R1 | Names | ¹H NMR | M + H |
| 42 | 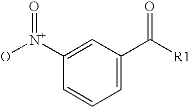 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-nitro-benzamide | 1H NMR (500 MHz, DMSO-d6) δ 8.74 (1H, d), 8.71 (1H, s), 8.37 (2H, dd), 8.32 (1H, d), 8.20 (1H, dd), 7.79 (1H, t), 5.31-5.05 (1H, m), 4.80 (1H, s), 3.97 (1H, s), 2.80-2.64 (6H, m), *(2H in solvent peak), 2.10 (2H, d), 1.93 (2H, dd), 1.66-1.53 (2H, m), 1.45 (2H, d). | 528 |
| 43 | 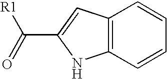 | N-[4-9-fluoro-2,4-dioxo-5-tetrahydrothiopyran-4-yl-3,5,7-triazabicyclo[4.4.0]deca-6,8,10-trien-3-yl)cyclohexyl]-1H-indole-2-carboxamide | 1H NMR (500 MHz, DMSO-d6) d 11.57 (1H, s), 8.74 (1H, d), 8.21 (1H, dd), 7.82 (1H, d), 7.64 (1H, d), 7.41 (1H, d), 7.29 (1H, d), 7.15 (1H, dt), 7.02 (1H, t), 5.32-5.05 (1H, m), 4.83-4.75 (1H, m), 4.01 (1H, s), 2.81-2.61 (6H, m), *(2H in solvent peak), 2.08 (2H, d), 1.93 (2H, dd), 1.61 (2H, t), 1.47 (2H, d). | 5.22 |
| 44 | 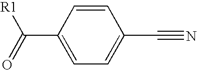 | 4-cyano-N-[4-(9-fluoro-2,4-dioxo-5-tetrahydrothiopyran-4-yl-3,5,7-triazabicyclo[4.4.0]deca-6,8,10-trien-3-yl)cyolohexyl]benzamide | 1H NMR (500 MHz, DMSO-d6) δ 8.74 (1H, d), 8.24 (1H, d), 8.21 (1H, dd), 8.03 (2H, d), 7.98 (2H, d), 5.33-5.07 (1H, m) 4.79 (1H, dd), 3.95 (1H, s), 2.80-260 (6H, m), *(2H in solvent peak), 2.07 (2H, d), 1.93 (2H, dd), 1.58 (2H, t), 1.44 (2H, d). | 508 |
| 45 | 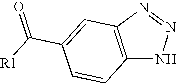 | N-[4-(9-fluoro-2,4-dioxo-5-tetrahydrothiopyran-4-yl-3,5,7-triazabicyclo[4.4.0]-deca-6,8,10-trien-3-yl)cyclohexyl]-1H-benzotriazole-5-carboxamide | 1H NMR (400 MHz, DMSO-d6) δ 8.79 (1H, d), 8.59 (1H, s), 8.27 (1H, dd), 8.19 (1H, d), 7.99 (1H, s), 5.23 (1H, s), 4.84 (1H, t), 4.03 (1H, s), 2.88-2.68 (8H, m), 2.15 (2H, d), 1.98 (2H, t), 1.64 (2H, t), 1.50 (2H, d). | 5.24 |
| 46 | 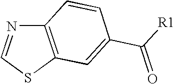 | N-[4-(9-fluoro-2,4-dioxo-5-tetrahydrothiopyran-4-yl-3,5,7-triazabicyclo[4.4.0]deca-6,8,10-trien-3-yl)cyclohexyl]benzothiazole-6-carboxamide | 1H NMR (500 MHz, DMSO-d6) δ 9.51 (1H, s), 8.74 (1H, d), 8.69 (1H, d), 8.21 (1H, dd), 8.17 (1H, d), 8.09 (1H, d), 8.05 (1H, dd), 5.30-5.09 (1H, m), 4.81 (1H, dd), 3.98 (1H, s), 2.80-2.65 (6H, m), *(2H in solvent peak), 2.11 (2H, d), 1.94 (2H, dd), 1.63-1.55 (2H, m), 1.46 (2H, d). | 540 |
| 47 | 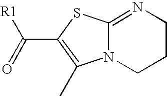 | N-[4-(3-fluoro-8,10-dioxo-7-tetrahydrothiopyran-4-yl-5,7,9-triazabicyclo[4.4.0]deca-13,5-trien-9-yl)cyclohexyl]-9-methyl-7-thia-1,5-diazabicyclo[4.3.0]nona-5,8-diene-8-carboxamide | 1H NMR (500 MHz, DMSO-d6) d10.42 (1H, s), 8.74 (1H, d), 8.15 (1H, dd), 7.97 (1H, d), 5.25-5.12 (1H, m), 4.81-4.70 (1H, m), 4.02 (2H, t), 3.90 (1H, s), 3.51-3.46 (1H, m), 2.79-2.66 (6H, m), *(5H in solvent peak), 2.11-2.04 (2H, m), 2.04-1.96 (2H, m), 1.96-1.88 (2H, m), 1.60-1.50 (2H, m), 1.48-1.39 (2H, m). | 559 |
| 48 | 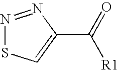 | N-[4-(9-fluoro-2,4-dioxo-5-tetrahydrothiopyran-4-yl-3,5,7-triazabicyclo[4.4.0]deca-6,8,10-trien-3-yl)cyclohexyl]thiadiazole-4-carboxamide | 1H NMR (500 MHz, DMSO-d6) d9.67 (1H, s), 8.73 (1H, d), 8.28 (1H, d), 8.21 (1H, dd), 5.32-5.02 (1H, m), 4.80 (1H, dd), 4.15 (1H, t), 2.81-2.64 (6H, m), *(2H in solvent peak), 2.04 (2H, d), 1.93 (2H, dd), 1.67 (2H, dd), 1.51 (2H, d). | 491 |
| 49 | 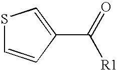 | N-[4-(9-fluoro-2,4-dioxo-5-tetrahydrothiopyran-4-yl-3,5,7-triazabicyclo[4.4.0]deca-6,8,10-trien-3-yl)cyclohexyl]thiophene-3-carboxamide | 1H NMR (500 MHz, DMSO-d6) d8.73 (1H, d), 8.23 (1H, dd), 8.20 (1H, dd), 7.68 (1H, d), 7.58-7.54 (2H, m), 5.31-5.03 (1H, m), 4.77 (1H, tt), 3.92 (1H, s) 2.87-2.64 (6H, m), *(2H in solvent peak), 2.05 (2H, d), 1.93 (2H, dd), 1.56 (2H, dd), 1.43 (2H, d). | 489 |
| 50 | 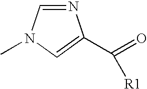 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-methyl-imidazole-4-carboxamide | 1H NMR (500 MHz, DMSO-d6) δ 8.74 (1H, d), 8.20 (2H, dd), 7.89 (1H, s), 7.75 (1H, s), 5.27-5.08 (1H, m), 4.80-4.70 (1H, m), 4.04 (1H, t), 3.75 (3H, s), 2.80-2.64 (6H, m), *(2H in solvent peak), 1.96-1.86 (4H, m), 1.63 (2H, t), 1.49 (2H, d). | |
| 51 | 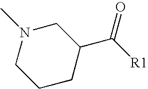 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-methyl-piperidine-3-carboxamide | 1H NMR (500 MHz, DMSO-d6) δ 9.29 (1H, s), 8.74 (1H, d), 8.17 (1H, dd), 7.98 (1H, d), 5.29-5.05 (1H, m), 4.70 (1H, dd), 3.73 (1H, s), 3.06-2.92 (1H, m), 2.90-2.62 (9H, m), *(5H in solvent peak), 2.00-1.32 (12H, m). | 504 |
| 52 | 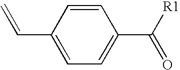 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-vinyl-benzamide | 1H NMR (500 MHz, DMSO-d6) δ 8.74 (1H, d), 8.21 (1H, dd), 7.88 (3H, d), 7.58 (2H, d), 6.78 (1H, dd), 5.93 (1H, d), 5.34 (1H, d), 5.29-5.08 (1H, m), 4.79 (1H, tt), 3.96 (1H, s), 2.80-2.62 (6H, m), *(2H in solvent peak), 2.06 (2H, d), 1.94 (2H, dd), 1.56 (2H, t), 1.44 (2H, d). | 509 |
| 53 | 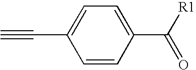 | 4-ethynyl-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide | 1H NMR (500 MHz, DMSO-d6) δ 8.73 (1H, d), 8.21 (1H, dd), 8.02 (1H, d), 7.89 (2H, d), 7.58 (2H, d), 5.33-5.03 (1H, m), 5.31-5.06 (1H, m), 4.84-4.71 (1H, m), 3.94 (1H, s), 2.84-2.60 (6H, m), *(2H in solvent peak), 2.06 (2H, d), 1.93 (2H, q), 1.56 (2H, t), 1.43 (2H, d). | 507 |
| 54 | 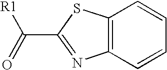 | N-[4-(9-fluoro-2,4-dioxo-5-tetrahydrothiopyran-4-yl-3,5,7-triazabicyclo[4.4.0]deca-6,8,10-trien-3-yl)cyclohexyl]benzothiazole-2-carboxamide | 1H NMR (500 MHz, DMSO-d6) δ 8.74 (1H, d), 8.31 (1H, d), 8.21 (3H, m), 7.63 (1H, dd), 7.57 (1H, dd), 5.32-5.04 (1H, m), 4.81 (1H, t), 4.11 (1H, t), 2.83-2.61 (6H, m), *(2H in solvent peak), 2.07 (2H, d), 1.94 (2H, dd), 1.67 (2H, t), 1.52 (2H, d). | 540 |
| 55 | 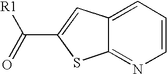 | N-[4-(9-fluoro-2,4-dioxo-5-tetrahydrothiopyran-4-yl-3,5,7-triazabicyclo[4.4.0]deca-6,8,10-trien-3-yl)cyclohexyl]-9-thia-2-azabicyclo[4.3.0]nona-1,3,5,7-tetraene-8-carboxamide | 1H NMR (500 MHz, DMSO-d6) δ 8.73 (1H, d), 8.62 (1H, dd), 8.41 (1H, dd), 8.33 (1H, d), 8.30 (1H, s), 8.20 (1H, dd), 7.48 (1H, dd), 5.31-5.06 (1H, m), 4.78 (1H, ddd), 3.97 (1H, s), 2.82-2.60 (6H, m), *(2H in solvent peak), 2.10 (2H, d), 1.93 (2H, dd), 1.61 (2H, t), 1.47 (2H, d). | 540 |
| 56 | 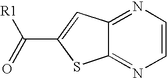 | N-[4-(9-fluoro-2,4-dioxo-5-tetrahydrothiopyran-4-yl-3,5,7-triazabicyclo[4.4.0]deca-6,8,10-trien-3-yl)cyclohexyl]-9-thia-2,5-diazabicyclo[4.3.0]nona-1,3,5,7-tetraene-8-carboxamide | 1H NMR (500 MHz, DMSO-d6) δ 8.82 (1H, d), 8.73 (1H, d), 8.68 (1H, d), 8.56 (1H, d), 8.51 (1H, d), 8.20 (1H, dd), 5.28-5.08 (1H, m), 4.78 (1H, ddd), 3.99 (1H, s), 2.79-2.61 (6H, m), *(2H in solvent peak), 2.12 (2H, d), 1.92 (2H, dd), 1.66-1.58 (2H, m), 1.47 (2H, d). | 541 |
| 57 | 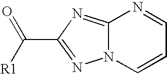 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1,5,7,9-tetrazabicyclo[4.3.0]nona-2,4,6,8-tetraene-8-carboxamide | 1H NMR (500 MHz, DMSO-d6) δ 9.46 (1H, dd), 8.98 (1H, dd), 8.73 (1H, d), 8.22 (1H, dd), 8.11 (1H, d), 7.46 (1H, dd), 5.32-5.05 (1H, m), 4.80 (1H, t), 4.15 (1H, t), 2.81-2.62 (6H, m), *(2H in solvent peak), 2.00 (2H, d), 1.93 (2H, dd), 1.68 (2H, t), 1.53 (2H, d). | 525 |
| 58 | 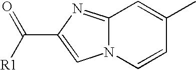 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-methyl-1,7-diazabicyclo[4.3.0]nona-2,4,6,8-tetraene-8-carboxamide | 1H NMR (300 MHz, DMSO-d6) δ 8.78 (1H, d), 8.47 (1H, d), 8.31 (1H, d), 8.25 (1H, dd), 7.75 (1H, d), 7.48 (1H, s), 6.85 (1H, dd), 5.03-5.45 (1H, bs), 4.84 (1H, t), 4.17 (1H, bs), 2.51-2.89 (8H, m), 2.38 (3H, s), 1.91-2.03 (4H, m),1.51-1.77 (4H, m). | 537 |
| 59 | 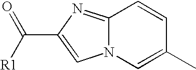 | N-[4-(3-fluoro-8,10-dioxo-7-tetrahydrothiopyran-4-yl-5,7,9-triazabicyclo[4.4.0]deca-1,3,5-trien-9-yl)cyclohexyl]-3-methyl-1,7-diazabicyclo[4.3.0]nona-2,4,6,8-tetraene-8-carboxamide | 1H NMR (500 MHz, DMSO-d6) δ 8.78 (1H, d), 8.40 (1H, s), 8.30 (1H, s), 8.25 (1H, dd), 7.73 (1H, d), 7.63 (1H, dd), 7.25 (1H, dd), 5.04-5.39 (1H, bs), 4.84 (1H, t), 4.17 (1H, dd), 2.67-2.87 (6H, m), 2.53-2.67 (2H, m), 2.30 (3H, s), 1.92-2.02 (4H, m), 1.63-1.77 (2H, m), 1.52-1.63 (2H, m). | 537 |
| 60 | 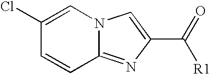 | 3-chloro-N-[4-(3-fluoro-8,10-dioxo-7-tetrahydrothiopyran-4-yl-5,7,9-triazabicyclo[4.4.0]deca-1,3,5-trien-9-yl)cyclohexyl]-1,7-diazabicyclo[4.3.0]nona-2,4,6,8-tetraene-8-carboxamide | 1H NMR (400 MHz, DMSO-d6) d8.91 (1H, d), 8.79 (1H, d), 8.39 (1H, s), 8.25 (1H, dd), 7.79 (2H, d), 7.47 (1H, dd), 5.23 (1H, bs), 4.83 (1H, t), 4.17 (1H, s), 2.81-2.70 (6H, m), 2.60 (2H, d), 2.02-1.95 (4H, m), 1.71 (2H, t), 1.57 (2H, d). | 557 |
| 61 | 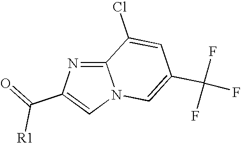 | 5-chloro-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(trifluoromethyl)-1,7-diazabicyclo[4.3.0]nona-2,4,6,8-tetraene-8-carboxamide | 1H NMR (400 MHz, DMSO-d6) d9.30 (1H, s), 8.79 (1H, d), 8.59 (1H, s), 7.98 (1H, s), 7.89-7.85 (1H, m), 5.22 (1H, bs), 4.87 (1H, t), 4.23 (1H, s), 2.83-2.57 (8H, m), 1.98 (4H, d), 1.72 (2H, t), 1.59 (2H, d). | 625 |
| 62 | 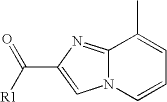 | N-[4-(9-fluoro-2,4-dioxo-5-tetrahydrothiopyran-4-yl-3,5,7-triazabicyclo[4.4.0]deca-6,8,10-trien-3-yl)cyclohexyl]1,5,7-triazabicyclo[4.3.0]nona-2,4,6,8-tetraene-8-carboxamide | 1H NMR (400 MHz, DMSO-d6) δ 8.79 (1H, d); 8.56-8.50 (2H, m); 8.23 (1H, dd); 8.15-8.10 (1H, m); 7.32 (1H, s); 7.03 (1H, s); 5.22 (1H, bs); 4.87 (1H, t); 4.20 (1H, s); 2.83-2.69 (6H, m), 2.65-2.59 (5H, m); 2.02-1.94 (4H, m); 1.71 (2H, t); 1.58 (2H, d). | 524 |
| 63 | 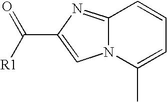 | N-[4-(9-fluoro-2,4-dioxo-5-tetrahydrothiopyran-4-yl-3,5,7-triazabicyclo[4.4.0]deca-6,8,10-trien-3-yl)cyclohexyl]-5-methyl-1,7-diazabicyclo[4.3.0]nona-2,4,6,8-tetraene-8-carboxamide | 1H NMR (400 MHz, DMSO-d6) δ 8.79 (1H, d), 8.56-8.50 (2H, m), 8.23 (1H, dd), 8.15-8.10 (1H, m), 7.32 (1H, s), 7.03 (1H, s), 4.87 (1H, t), 5.22 (1H, bs), 4.20 (1H, s), 2.83-2.69 (6H, m), 2.65-2.59 (2H, m), 2.3 (3H, s) 2.02-1.94 (4H, m), 1.71 (2H, t), 1.58 (2H, d). | 537 |
| 64a | 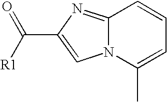 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-1,7-diazabicyclo[4.3.0]nona-2,4,6,8-tetraene-8-carboxamide | 1H NMR (400 MHz, DMSO-d6) δ 8.79 (1H, d); 8.54 (1H, s); 8.25 (1H, dd); 8.05 (1H, s); 7.67 (1H, d); 7.54 (1H, s); 7.07 (1H, s); 5.22 (1H, bs); 4.84 (1H, t); 4.17 (1H, s); 2.82-2.58 (11H, m); 2.07-1.95 (4H, m); 1.73 (2H, t); 1.58 (2H, d). | 537 |

TABLE 2

| Example | | | |
|---|---|---|---|
| Number | R1 | Name | (M + H) |
| 65 | 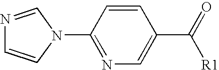 | N-[4-(9-fluoro-2,4-dioxo-5-tetrahydrothiopyran-4-yl-3,5,7-triazabicyclo[4.4.0]deca-6,8,10-trien-3-yl)cyclohexyl]-6-imidazol-1-yl-pyridine-3-carboxamide | 550 |
| 66 | 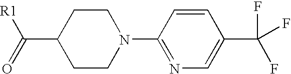 | N-[4-(9-fluoro-2,4-dioxo-5-tetrahydrothiopyran-4-yl-3,5,7-triazabicyclol[4.4.0]deca-6,8,10-trien-3-yl)cyclohexyl]-1-[5-(trifluoromethyl)-2-pyridyl]piperidine-4-carboxamide | 635 |
| 67 | 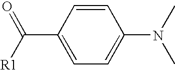 | 4-dimethylamino-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide | 526 |
| 68 | 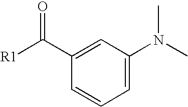 | 3-dimethylamino-N-[4-(9-fluoro-2,4-dioxo-5-tetrahydrothiopyran-4-yl-3,5,7-triazabicylo[4.4.0]deca-6,8,10-trien-3-yl)cyclohexyl]benzamide | 526 |
| 69 | 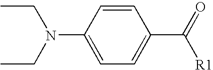 | 4-diethylamino-N-[4-(9-flooro-2,4-dioxo-5-tetrahydrothiopyran-4-yl-3,5,7-triazabicyclo[4.4.0]deca-6,8,10-trien-3-yl)cyclohexyl]benzamide | 554 |
| 70 | 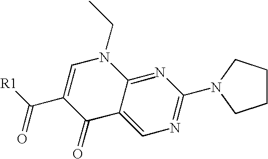 | 10-ethyl-N-[4-(9-fluoro-2,4-dioxo-5-tetrahydrothiopyran-4-yl-3,5,7-triazabicylo[4.4.0]deca-6,8,10-trien-3-yl)cyclohexyl]-7-oxo-3-pyrrolidin-1-yl-2,4,10-triazabicylco-[4.4.0]deca-1,3,5,8-tetraene-8-carboxamide | 649 |
| 71 | 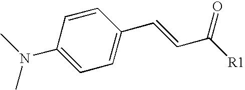 | (E)-3-(4-dimethylaminophenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]prop-2-enamide | 552 |
| 72 | 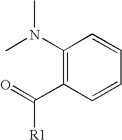 | 2-dimethylamino-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide | 526 |
| 73 | 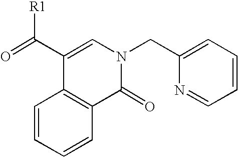 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-oxo-2-(2-pyridylmethyl)isoquinoline-4-carboxamide | 641 |
| 74 | 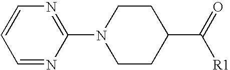 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-pyrimidin-2-yl-piperidine-4-carboxamide | 568 |
| 75 | 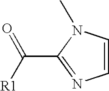 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-methyl-imidazole-2-carboxamide | 487 |
| 76 | 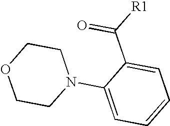 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-morpholino-benzamide | 568 |
| 77 | 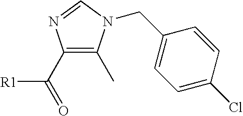 | 1-[(4-chlorophenyl)methyl]-N-[4-(9-fluoro-2,4-dioxo-5-tetrahydrothiopyran-4-yl-3,5,7-triazabicyclo[4.4.0]deca-6,8,10-trien-3-yl)cyclohexyl]-5-methyl-imidazole-4-carboxamide | 611 |
| 79 | 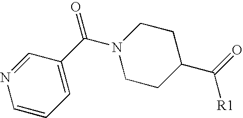 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-(pyridine-3-carbonyl)piperidine-4-carboxmide | 595 |
| 80 | 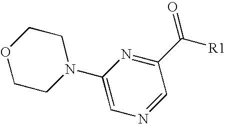 | N-[4-(9-fluoro-2,4-dioxo-5-tetrahydrothiopyran-4-yl-3,5,7-triazabicyclo[4.4.0]deca-6,8,10-trien-3-yl)cyclohexyl]-6-morpholino-pyrazine-2-carboxamide | 570 |
| 81 | 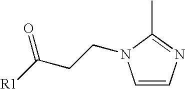 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(2-methylimidazol-1-yl)propanamide | 515 |
| 82 | 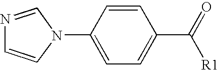 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-imidazol-1-yl-benzamide | 549 |
| 83 | 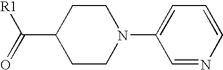 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-(3-pyridyl)piperidine-4-carboxamide | 567 |
| 84 | 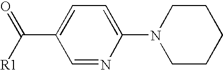 | N-[4-(9-fluoro-2,4-dioxo-5-tetrahydrothiopyran-4-yl-3,5,7-triazabicyclo[4.4.0]deca-6,8,10-trien-3-yl)cyclohexyl]-6-(1-piperidyl)pyridine-3-carboxamide | 567 |
| 85 | 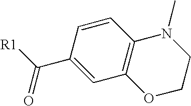 | N-[4-(9-fluoro-2,4-dioxo-5-tetrahydrothiopyran-4-yl-3,5,7-triazabicyclo[4.4.0]deca-6,8,10-trien-3-yl)cyclohexyl]-5-methyl-2-oxa-5-azabicyclo[4.4.0]deca-7,9,11-triene-9-carboxamide | 554 |
| 86 | 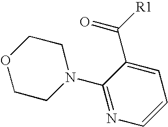 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-morpholino-pyridine-3-carboxamide | 569 |
| 87 | 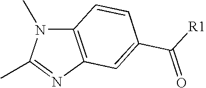 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1,2-dimethyl-benzoimidazole-5-carboxamide | 551 |
| 88 | 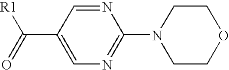 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-morpholino-pyrimidine-5-carboxamide | 570 |
| 89 | 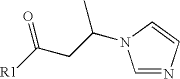 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-imidazol-1-yl-butanamide | 515 |
| 90 | 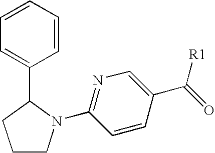 | N-[4-(9-fluoro-2,4-dioxo-5-tetrahydrothiopyran-4-yl-3,5,7-triazabicyclo[4.4.0]deca-6,8,10-trien-3-yl)cyclohexyl]-6-(2-phenylpyrrolidin-1-yl)pyridine-3-carboxamide | 629 |
| 91 | 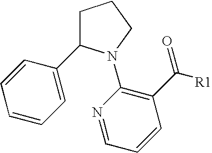 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(2-phenylpyrrolidin-1-yl)pyridine-3-carboxamide | 629 |
| 93 | 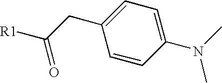 | 2-(4-dimethylaminophenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 540 |
| 94 | 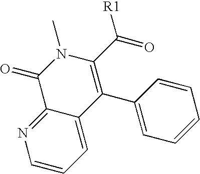 | N-[4-(9-fluoro-2,4-dioxo-5-tetrahydrothiopyran-4-yl-3,5,7-triazabicyclo[4.4.0]deca-6,8,10-trien-3-yl)cyclohexyl]-7-methyl-8-oxo-5-phenyl-1,7-naphthyridine-6-carboxamide | 641 |
| 95 | 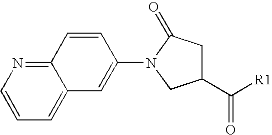 | N-[4-(9-fluoro-2,4-dioxo-5-tetrahydrothiopyran-4-yl-3,5,7-triazabicyclo[4.4.0]deca-6,8,10-trien-3-yl)cyclohexyl]-5-oxo-1-(6-quinolyl)pyrrolidine-3-carboxamide | 617 |
| 96 | 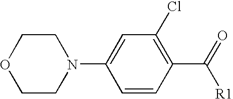 | 2-chloro-N-[4-(9-fluoro-2,4-dioxo-5-tetrahydrothiopyran-4-yl-3,5,7-triazabicyclo[4.4.0]deca-6,8,10-trien-3-yl)cyclohexyl]-4-morpholino-benzamide | 602 |
| 97 | 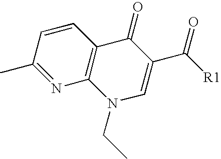 | 1-ethyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-7-methyl-4-oxo-1,8-naphthyridine-3-carboxamide | 593 |
| 98 | 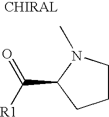 | (2S)-N-[4-(9-fluoro-2,4-dioxo-5-tetrahydrothiopyran-4-yl-3,5,7-triazabicyclo[4.4.0]deca-6,8,10-trien-3-yl)cyclohexyl]-1-methyl-pyrrolidine-2-carboxamide | 490 |
| 99 | 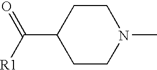 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-methyl-piperidine-4-carboxamide | 504 |
| 100 | 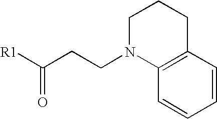 | 3-(3,4-dihydro-2H-quinolin-1-yl)-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide | 566 |
| 101 | 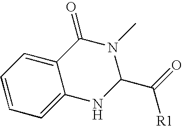 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methyl-4-oxo-1,2-dihydroquinazoline-2-carboxamide | 567 |
| 102 | 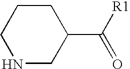 | N-[4-(9-fluoro-2,4-dioxo-5-tetrahydrothiopyran-4-yl-3,5,7-triazabicyclo[4.4.0]deca-6,8,10-trien-3-yl)cyclohexyl]piperidine-3-carboxamide | 490 |
| 103 | 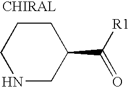 | (3R)-N-[4-(9-fluoro-2,4-dioxo-5-tetrahydrothiopyran-4-yl-3,5,7-triazabicyclo[4.4.0]deca-6,8,10-trien-3-yl)cyclohexyl]piperidine-3-carboxamide | 490 |
| 104 | 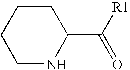 | N-[4-(9-fluoro-2,4-dioxo-5-tetrahydrothiopyran-4-yl-3,5,7-triazabicyclo[4.4.0]deca-6,8,10-trien-3-yl)cyclohexyl]piperidine-2-carboxamide | 490 |
| 105 | 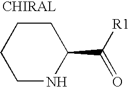 | (2S)-N-[4-(9-fluoro-2,4-dioxo-5-tetrahydrothiopyran-4-yl-3,5,7-triazabicyclo[4.4.0]deca-6,8,10-trien-3-yl)cyclohexyl]piperidine-2-carboxamide | 490 |
| 106 | 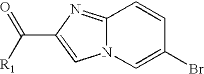 | 3-bromo-N-[4-(9-fluoro-2,4-dioxo-5-tetrahydrothiopyran-4-yl-3,5,7-triazabicyclo[4.4.0]deca-6,8,10-trien-3-yl)cyclohexyl]-1,7-diazabicyclo[4.3.0]nona-2,4,6,8-tetraene-8-carboxamide | 601/603 |
| 107a | 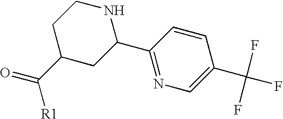 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-[5-(trifluoromethyl)-2-pyridyl]piperidine-4-carboxamide | 635 |
| 108a | 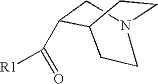 | N-[4-(9-fluoro-2,4-dioxo-5-tetrahydrothiopyran-4-yl-3,5,7-triazabicyclo[4.4.0]deca-6,8,10-trien-3-yl)cyclohexyl]quinuclidine-3-carboxamide | 516 |
| 109a | 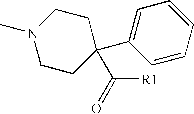 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-methyl-4-phenyl-piperidine-4-carboxamide | 580 |
| 110a | 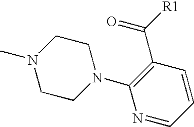 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-methylpiperazin-1-yl)pyridine-3-carboxamide | 582 |
| 111a | 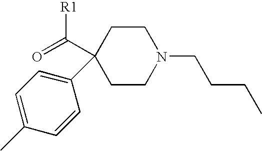 | 1-butyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(p-tolyl)piperidine-4-carboxamide | 636 |
| 112a | 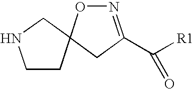 | N-[4-(9-fluoro-2,4-dioxo-5-tetrahydrothiopyran-4-yl-3,5,7-triazabicyclo[4.4.0]deca-6,8,10-trien-3-yl)cyclohexyl]-9-oxa-2,8-diazaspiro[4.4]non-7-ene-7-carboxamide | 531 |
| 113a | 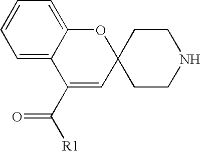 | N-[4-(9-fluoro-2,4-dioxo-5-tetrahydrothiopyran-4-yl-3,5,7-triazabicyclo[4.4.0]deca-6,8,10-trien-3-yl)cyclohexyl]carboxamide | 606 |
| 114a | 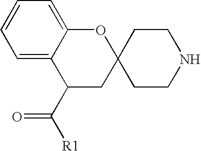 | N-[4-(9-fluoro-2,4-dioxo-5-tetrahydrothiopyran-4-yl-3,5,7-triazabicyclo[4.4.0]deca-6,8,10-trien-3-yl)cyclohexyl]carboxamide | 608 |
| 115a | 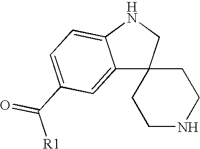 | N-[4-(9-fluoro-2,4-dioxo-5-tetrahydrothiopyran-4-yl-3,5,7-triazabicyclo[4.4.0]deca-6,8,10-trien-3-yl)cyclohexyl]carboxamide | 593 |
| 116a | 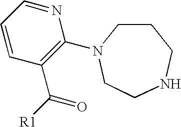 | 2-(1,4-diazepan-1-yl)-N-[4-(9-fluoro-2,4-dioxo-5-tetrahydrothiopyran-4-yl-3,5,7-triazabicyclo[4.4.0]deca-6,8,10-trien-3-yl)cyclohexyl]pyridine-3-carboxamide | 582 |
| 117a | 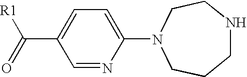 | 6-(1,4-diazepan-1-yl)-N-[4-(9-fluoro-2,4-dioxo-5-tetrahydrothiopyran-4-yl-3,5,7-triazabicyclo[4.4.0]deca-6,8,10-trien-3-yl)cyclohexyl]pyridine-3-carboxamide | 582 |

### EXAMPLE 118a

### N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-6-(1-methylpyrrolidin-2-yl)imidazo[1,2-a]pyridine-2-carboxamide

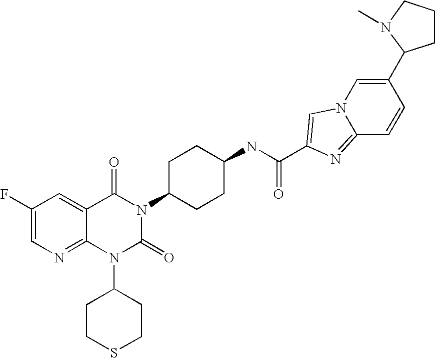

### Step (a) 6-(1-methylpyrrolidin-2-yl)imidazo[1,2-a]pyridine-2-carboxylic Acid

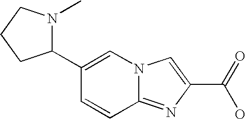

A mixture of 5-(1-Methyl-pyrrolidin-2-yl)-pyridin-2-ylamine (600 mg, 3 mmol), and ethylbromo pyruvate (1.17 g, 6.0 mmol) in ethanol (20 ml) was heated at 70° C. for 2 h. The mixture was evaporated to dryness and the residue triturated with acetonitrile (10 ml). The crude ester was collected by filtration and then dissolved in a mixture of water/dioxan (1:1) (20 ml). To this solution was added lithium hydroxide monohydrate (0.3 g, 7.14 mmol) and the mixture stirred at room temperature overnight. The mixture was evaporated to dryness and the residue taken up into water (20 ml) and the pH was adjusted to 4-5 by the addition of acetic acid. The solid was then collected by filtration and dried to afford the sub-title compound (310 mg, 42%).

¹H NMR (400 MHz, DMSO-d₆) δ 8.46 (s, 1H), 8.35 (s, 1H), 7.55 (d, 1H), 7.29 (dd, 1H), 3.12 (m, 2H), 2.50 (m, 3H), 2.27 (q, 1H), 2.17 (m, 1H), 1.75 (m, 3H)

### Step (b) N-{cis-4-[6-fluoro-2,4-dioxo-1-(tetrahydro-2H-thiopyran-4-yl)-1,4-dihydropyrido[2,3-d]pyrimidin-3(2H)-yl]cyclohexyl}-6-(1-methylpyrrolidin-2-yl)imidazo[1,2-a]pyridine-2-carboxamide

6-(1-Methyl-pyrrolidin-2-yl)-imidazo[1,2-a]pyridine-2-carboxylic acid (98 mg, 0.4 mmol) was dissolved in dry DMF (5 ml) and DIEA (0.2 ml, 1.15 mmol) was added, followed by HATU (160 mg, 0.42 mmol) and the mixture stirred for 10 min. 3-(4-Amino-cyclohexyl)-6-fluoro-1-(tetrahydro-thiopyran-4-yl)-1H-pyrido[2,3-d]pyrimidine-2,4-dione (150 mg, 0.4 mmol) was added and the mixture stirred at room temperature overnight. The mixture was poured into water and the solid that precipitated was collected by filtration and subsequently purified by reverse phase HPLC (25-95% acetonitrile in aqueous ammonia) to afford the title compound (94 mg, 39%).

¹H NMR (300 MHz, DMSO-d₆) δ 8.79 (d, 1H), 8.52 (s, 1H), 8.37 (s, 1H), 8.26 (dd, 1H), 7.74 (d, 1H), 7.68 (d, 1H), 7.36 (dd, 1H), 5.32 (s, 1H), 4.84 (t, 1H), 4.16 (s, 1H), 3.15 (dd, 2H), 2.72 (m, 6H), 2.28 (m, 2H), 2.14 (s, 3H), 1.97 (s, 4H), 1.68 (m, 9H)

APCI (Multimode) m/z: 606 [M+H]

TABLE 3

| Example No | COMPOUND | Name | (M + H) |
|---|---|---|---|
| 107 | 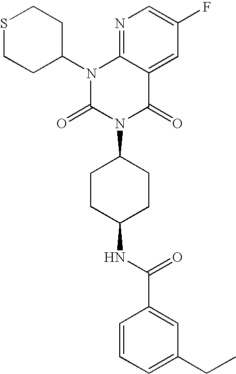 | 3-ethyl-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide | 511 |
| 108 | 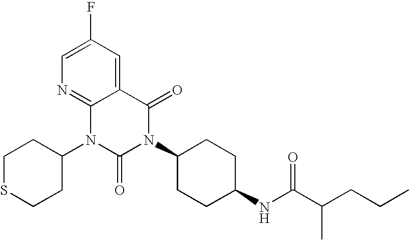 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-pentanamide | 477 |
| 109 | 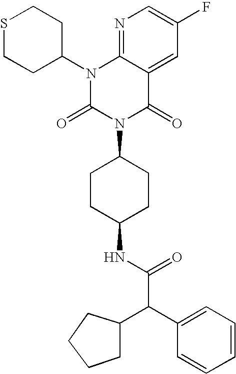 | 2-cyclopentyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-phenyl-acetamide | 565 |
| 110 | 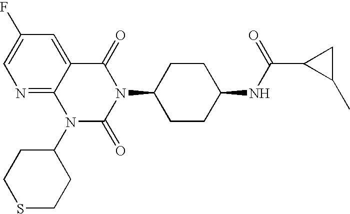 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-cyclopropane-1-carboxamide | 461 |
| 111 | 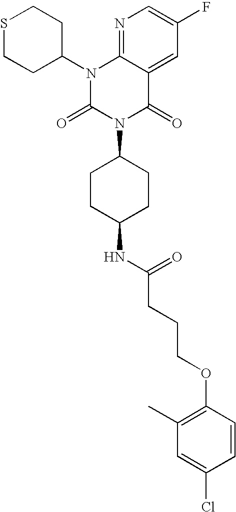 | 4-(4-chloro-2-methyl-phenoxy)-N-[4-(9-fluoro-2,4-dioxo-5-tetrahydrothiopyran-4-yl-3,5,7-triazabicyclo[4.4.0]deca-6,8,10-trien-3-yl)cyclohexyl]butanamide | 589 |
| 112 | 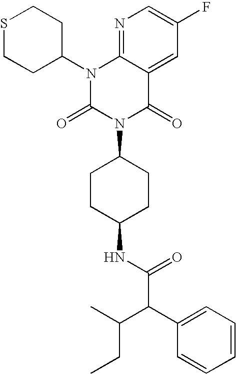 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methyl-2-phenyl-pentanamide | 553 |
| 113 | 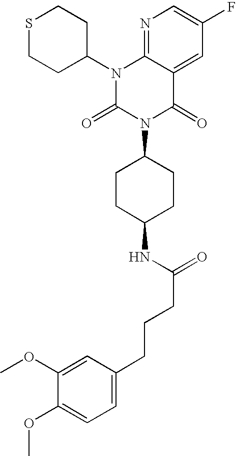 | 4-(3,4-dimethoxyphenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]butanamide | 585 |
| 114 | 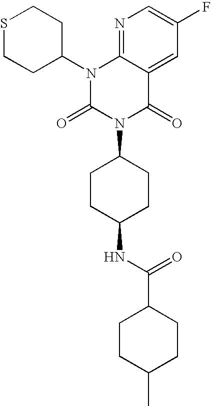 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-methyl-cyclohexane-1-carboxamide | 503 |
| 115 | 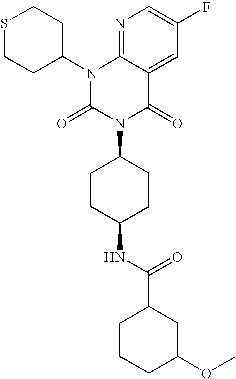 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methoxy-cyclohexane-1-carboxamide | 519 |
| 116 | 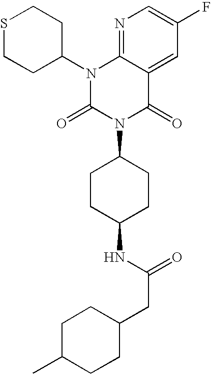 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-methylcyclohexyl)acetamide | 517 |
| 117 | 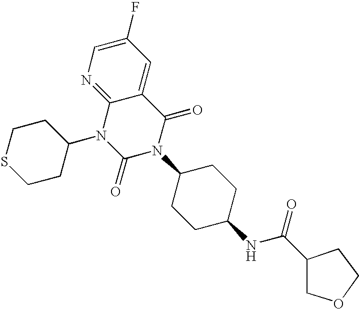 | N-[4-(9-fluoro-2,4-dioxo-5-tetrahydrothiopyran-4-yl-3,5,7-triazabicyclo[4.4.0]deca-6,8,10-trien-3-yl)cyclohexyl]tetrahydrofuran-3-carboxamide | 477 |
| 118 | 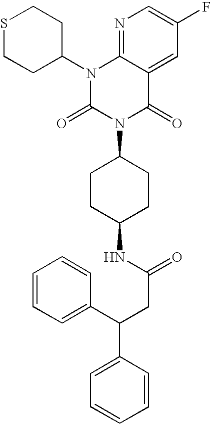 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3,3-diphenyl-propanamide | 587 |
| 119 | 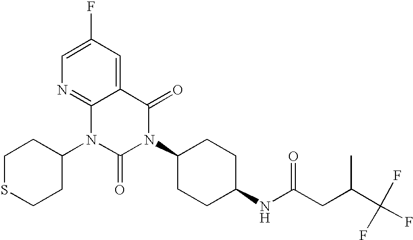 | 4,4,4-trifluoro-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methyl-butanamide | 517 |
| 120 | 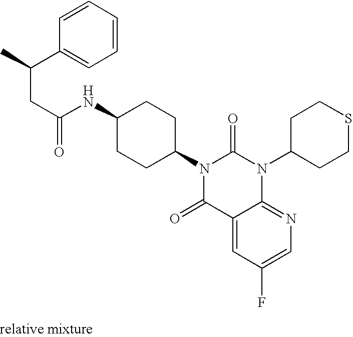 | (3R)-N-[4-(9-fluoro-2,4-dioxo-5-tetrahydrothiopyran-4-yl-3,5,7-triazabicyolo[4.4.0]deca-6,8,10-trien-3-yl)cyclohexyl]-3-phenyl-butanamide | 525 |
| 121 | 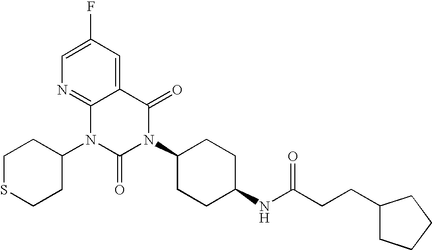 | 3-cyclopentyl-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide | 503 |
| 122 | 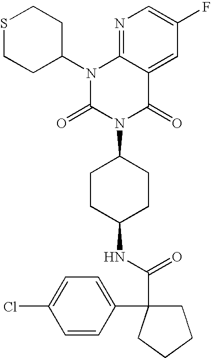 | 1-(4-chlorophenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]cyclopentane-1-carboxamide | 585 |
| 123 | 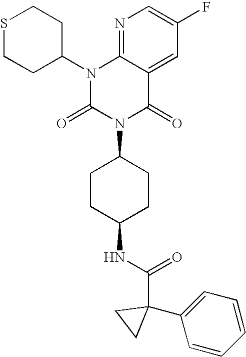 | N-[4-(9-fluoro-2,4-dioxo-5-tetrahydrothiopyran-4-yl-3,5,7-triazabicyclo[4.4.0]deca-6,8,10-trien-3-yl)cyclohexyl]1-1-phenyl-cyclopropane-1-carboxamide | 523 |
| 124 | 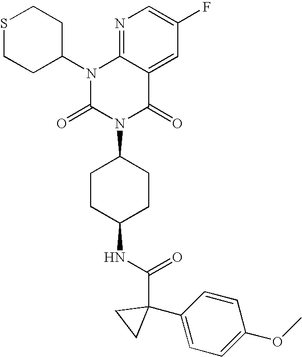 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-(4-methoxyphenyl)cyclopropane-1-carboxamide | 553 |
| 125 | 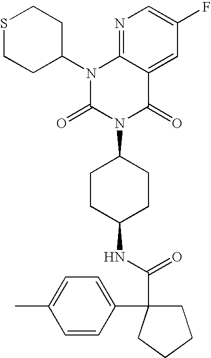 | N-[4-(9-fluoro-2,4-dioxo-5-tetrahydrothiopyran-4-yl-triazabicyclo[4.4.0]deca-6,8,10-trien-3-yl)cyclohexyl]-1-(p-tolyl)cyclopentane-1-carboxamide | 565 |
| 126 | 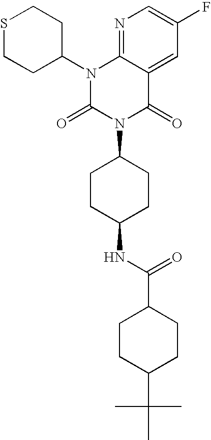 | N-[4-(9-fluoro-2,4-dioxo-5-tetrahydrothiopyran-4-yl-3,5,7-triazabicyclo]4.4.0]deca-6,8,10-trien-3-yl)cyclohexyl]-4-tert-butyl-cyclohexane-1-carboxamide | 545 |
| 127 | 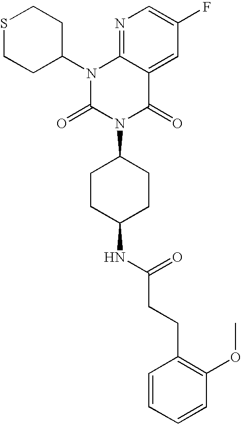 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(2-methoxyphenyl)propanamide | 541 |
| 128 | 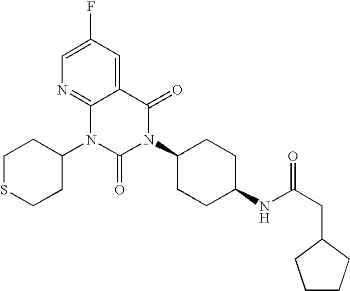 | 2-cyclopentyl-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 489 |
| 129 | 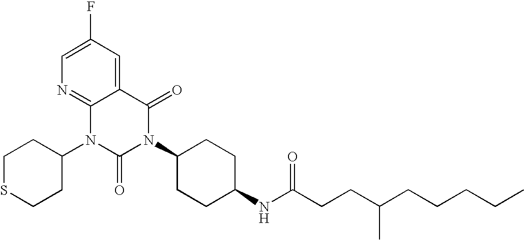 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-methyl-nonanamide | 533 |
| 130 | 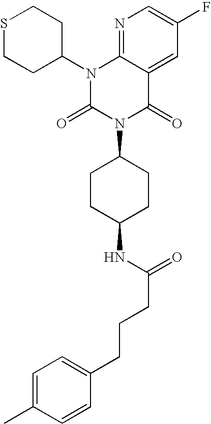 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(p-tolyl)butanamide | 539 |
| 131 | 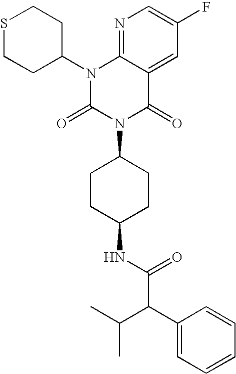 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methyl-2-phenyl-butanamide | 539 |
| 132 | 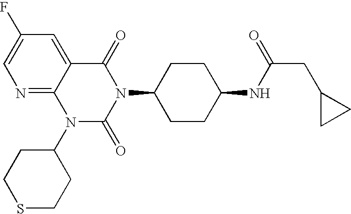 | 2-cyclopropyl-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 461 |
| 133 | 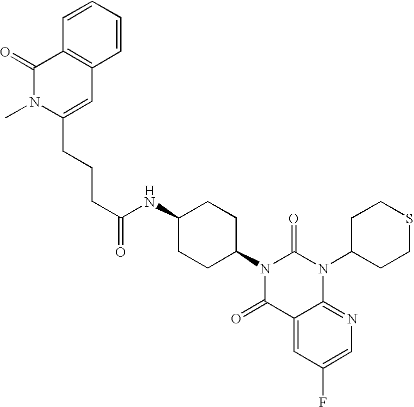 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(2-methyl-1-oxo-3-isoquinolyl)butanamide | 606 |
| 134 | 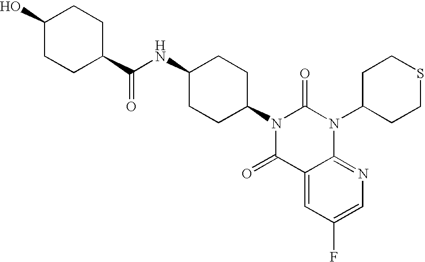 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-hydroxy-cyclohexane-1-carboxamide | 505 |
| 135 | 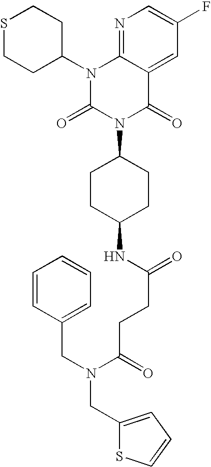 | N′-benzyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-N′-(2-thienylmethyl)butanediamide | 664 |
| 136 | 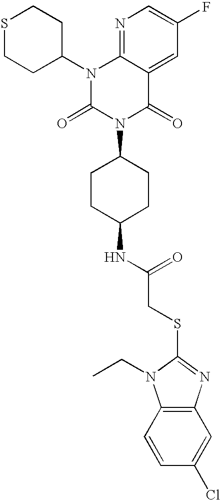 | 2-(5-chloro-1-ethyl-benzoimidazol-2-yl)sulfanyl-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 631 |
| 137 | 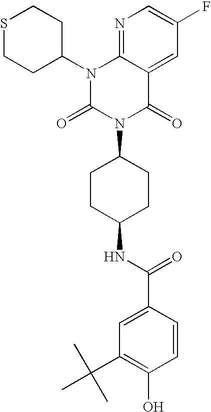 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-hydroxy-3-tert-butyl-benzamide | 555 |
| 139 | 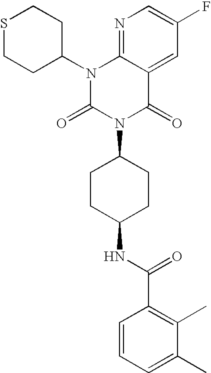 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2,3-dimethyl-benzamide | 511 |
| 140 | 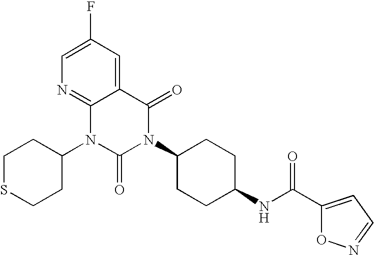 | N-[4-(9-fluoro-2,4-dioxo-5-tetrahydrothiopyran-4-yl-3,5,7-triazabicyclo[4.4.0]deca-6,8,10-trien-3-yl)cyclohexyl]isoxazole-5-carboxamide | 474 |
| 141 | 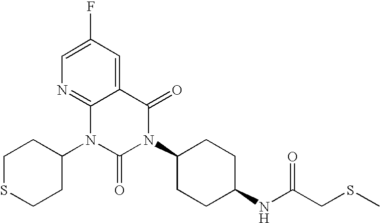 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methylsulfanyl-acetamide | 467 |
| 142 | 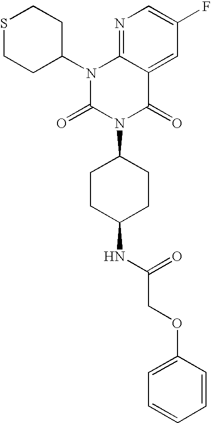 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-phenoxy-acetamide | 513 |
| 143 | 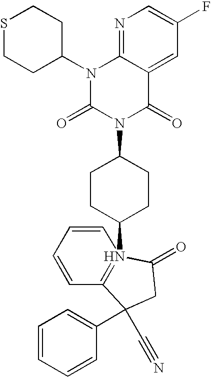 | 3-cyano-N-[4-(9-fluoro-2,4-dioxo-5-tetrahydrothiopyran-4-yl-3,5,7-triazabicyclo[4.4.0]deca-6,8,10-trien-3-yl)cyclohexyl]-3,3-diphenyl-propanamide | 612 |
| 144 | 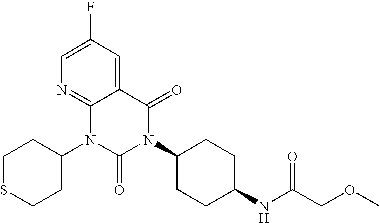 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methoxy-acetamide | 451 |
| 145 | 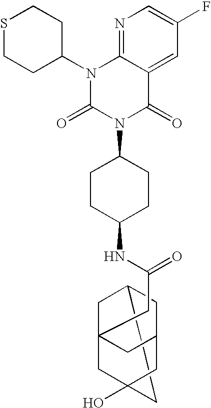 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(3-hydroxy-1-adamantyl)acetamide | 571 |
| 146 | 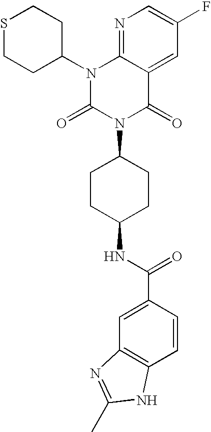 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-1H-benzoimidazole-5-carboxamide | 537 |
| 147 | 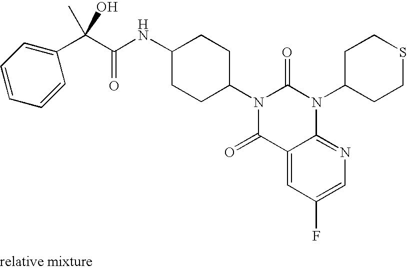 | (2R)-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-2-phenyl-propanamide | 527 |
| 149 | 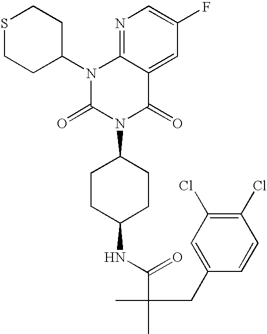 | 3-(3,4-dichlorophenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]-2,2-dimethyl-propanamide | 607 |
| 150 | 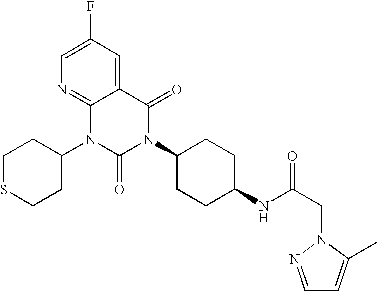 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(5-methylpyrazol-1-yl)acetamide | 501 |
| 151 | 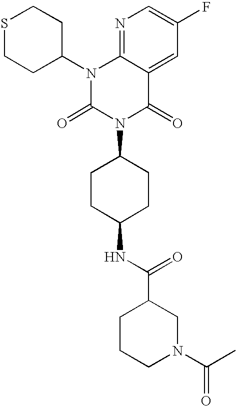 | 1-acetyl-N-[4-(9-fluoro-2,4-dioxo-5-tetrahydrothiopyran-4-yl-3,5,7-triazabicyclo]4.4.0]deca-6,8,10-trien-3-yl)cyclohexyl]piperidine-3-carboxamide | 532 |
| 152 | 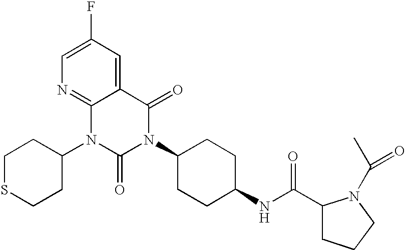 | 1-acetyl-N-[4-(9-fluoro-2,4-dioxo-5-tetrahydrothiopyran-4-yl-3,5,7-triazabicyclo[4.4.0]deca-6,8,10-trien-3-yl)cyclohexyl]pyrrolidine-2-carboxamide | 518 |
| 153 | 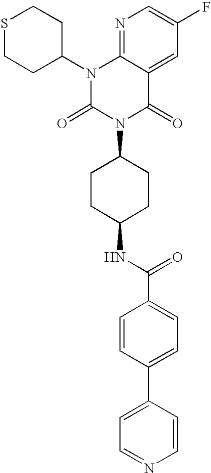 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(4-pyridyl)benzamide | 560 |
| 154 | 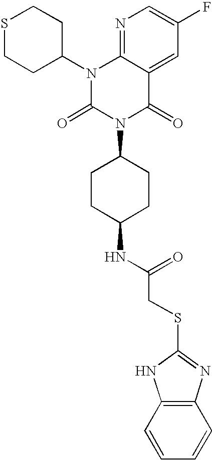 | 2-(1H-benzoimidazol-2-ylsulfanyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 569 |
| 155 | 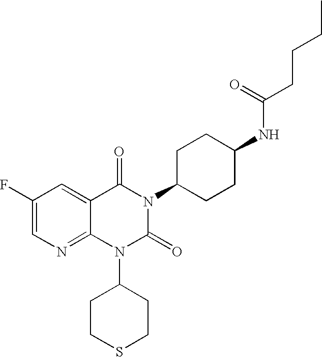 | N-[4-(9-fluoro-2,4-dioxo-5-tetrahydrothiopyran-4-yl-3,5,7-triazabicyclo[4.4.0]deca-6,8,10-trien-3-yl)cyclohexyl]pentanamide | 463 |
| 156 | 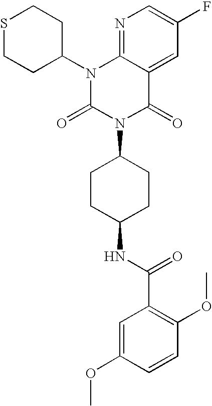 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2,5-dimethoxy-benzamide | 543 |
| 157 | 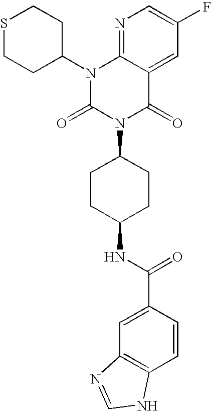 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1H-benzoimidazole-5-carboxamide | 523 |
| 158 | 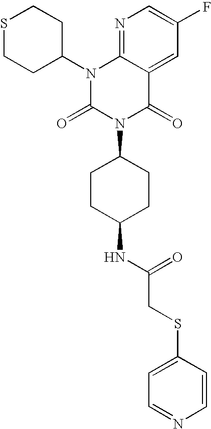 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-pyridylsulfanyl)acetamide | 530 |
| 159 | 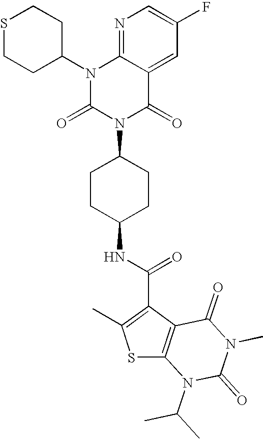 | N-[4-(9-fluoro-2,4-dioxo-5-tetrahydrothiopyran-4-yl-3,5,7-triazabicyclo[4.4.0]deca-6,8,10-trien-3-yl)cyclohexyl]-5-isopropyl-3,8-dimethyl-2,4-dioxo-7-thia-3,5-diazabicyclo[4.3.0]nona-8,10-diene-9-carboxamide | 643 |
| 160 | 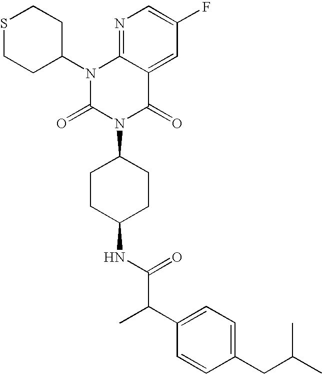 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-isobutylphenyl)propanamide | 567 |
| 161 | 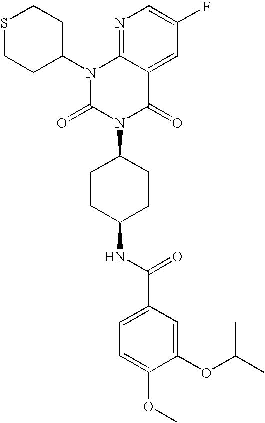 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-isopropoxy-4-methoxy-benzamide | 571 |
| 162 | 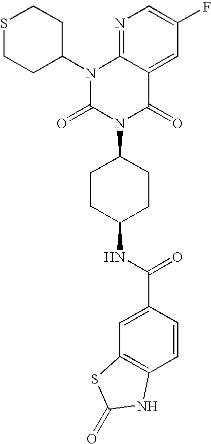 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-oxo-3H-benzothiazole-6-carboxamide | 556 |
| 163 | 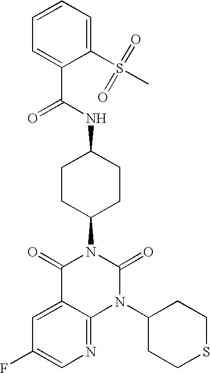 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methylsulfonyl-benzamide | 561 |
| 164 | 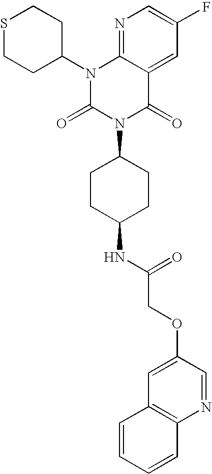 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(3-quinolyloxy)acetamide | 564 |
| 165 | 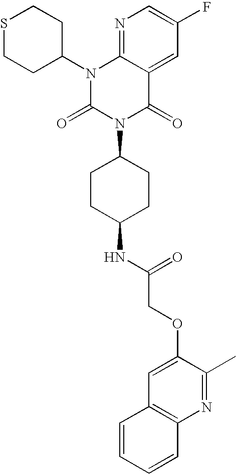 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-quinolinr-3-carboxamide | 548 |
| 166 | 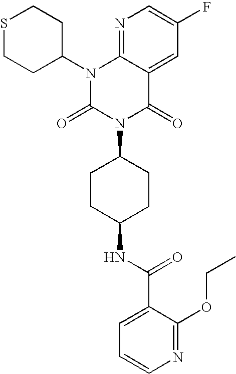 | 2-ethoxy-N-[4-(9-fluoro-2,4-dioxo-5-tetrahydrothiopyran-4-yl-3,5,7-triazabicyclo[4.4.0]deca-6,8,10-trien-3-yl)cyclohexyl]pyridine-3-carboxamide | 528 |
| 167 | 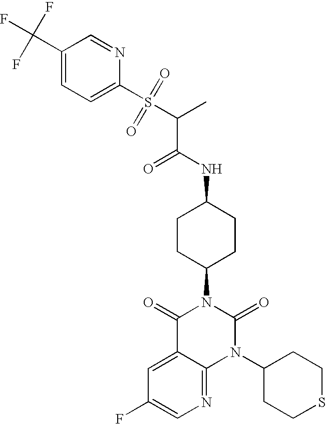 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-[[5-(trifluoromethyl)-2-pyridyl]sulfonyl]propanamide | 644 |
| 168 | 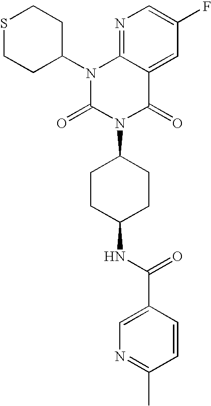 | N-[4-(9-fluoro-2,4-dioxo-5-tetrahydrothiopyran-4-yl-3,5,7-triazabicyclo[4.4.0]deca-6,8,10-trien-3-yl)cyclohexyl]-6-methyl-pyridine-3-carboxamide | 498 |
| 169 | 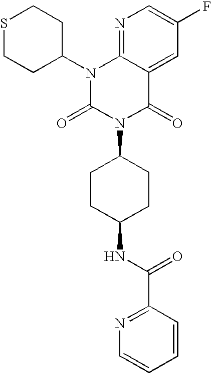 | N-[4-(9-fluoro-2,4-dioxo-5-tetrahydrothiopyran-4-yl-3,5,7-triazabicyclo[4.4.0]deca-6,8,10-trien-3-yl)cyclohexyl]pyridine-2-carboxamide | 484 |
| 170 | 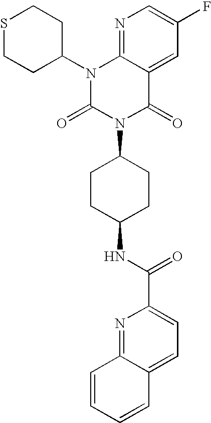 | N-[4-(9-fluoro-2,4-dioxo-5-tetrahydrothiopyran-4-yl-3,5,7-triazabicyclo[4.4.0]deca-6,8,10-trien-3-yl)cyclohexy]quinoline-2-carboxamide | 534 |
| 171 | 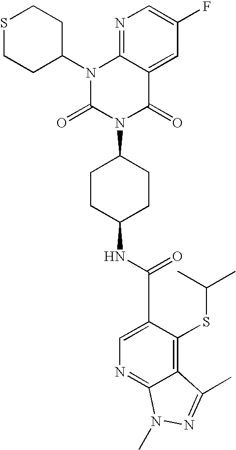 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-isopropylsulfanyl-7,9-dimethyl-5,7,8-triazabicyclo[4.3.0]nona-1,3,5,8-tetraene-3-carboxamide | 626 |
| 172 | 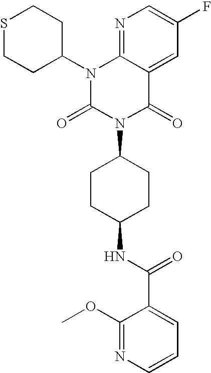 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methoxy-pyridine-3-carboxamide | 514 |
| 173 | 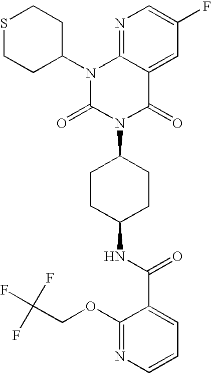 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(2,2,2-trifluoroethoxy)pyridine-3-carboxamide | 582 |
| 174 | 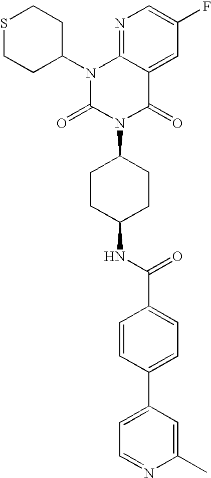 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(2-methyl-4-pyridyl)benzamide | 574 |
| 175 | 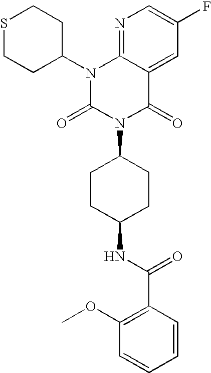 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methoxy-benzamide | 513 |
| 176 | 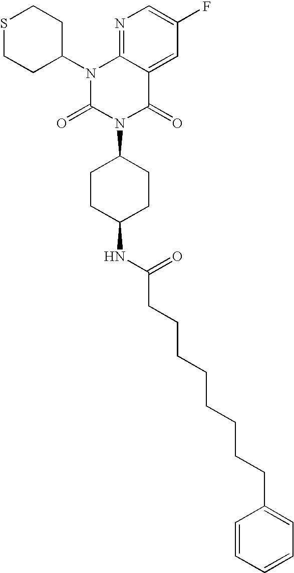 | N-[4-(9-fluoro-2,4-dioxo-5-tetrahydrothiopyran-4-yl-3,5,7-triazabicyclo[4.4.0]deca-6,8,10-trien-3-yl)cyolohexyl]-9-phenyl-nonanamide | 595 |
| 177 | 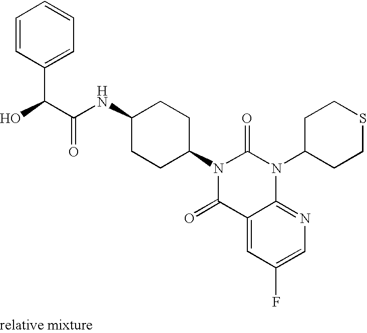 | (2S)-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-2-phenyl-acetamide | 513 |
| 178 | 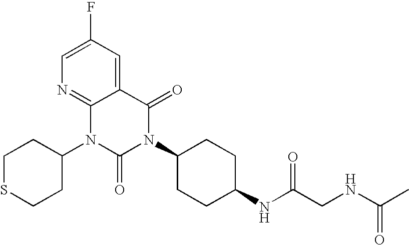 | 2-acetamido-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 478 |
| 179 | 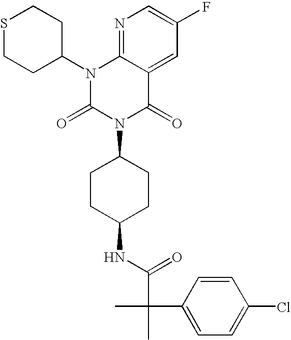 | 2-(4-chlorophenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-propanamide | 559 |
| 180 | 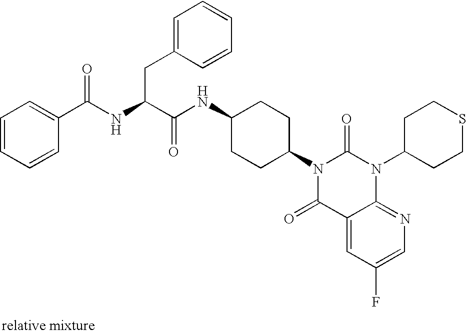 | N-[(1S)-1-[[4-(9-fluoro-2,4-dioxo-5-tetrahydrothiopyran-4-yl-3,5,7-ttriazabicyclo[4.40]deca-6,8,10-trien-3-yl)cyclohexyl]carbamoyl]-2-phenyl-ethyl]benzamide | 630 |
| 181 | 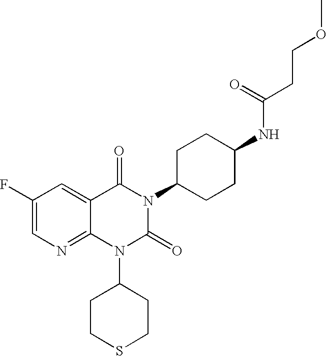 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methoxy-propanamide | 465 |
| 182 | 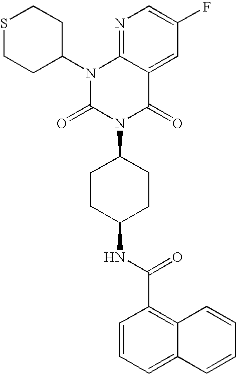 | N-[4-(9-fluoro-2,4-dioxo-5-tetrahydrothiopyran-4-yl-3,5,7-triazabicyclo[4.4.0]deca-6,8,10-trien-3-yl)cyclohexyl]naphthalene-1-carboxamide | 533 |
| 183 | 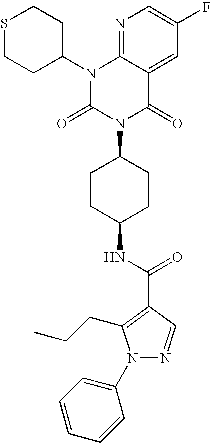 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-phenyl-5-propyl-pyrazole-4-carboxamide | 591 |
| 184 | 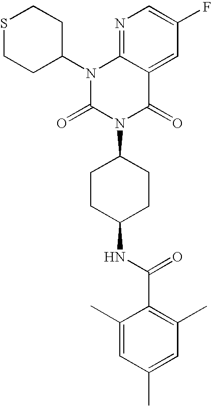 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2,4,6-trimethyl-benzamide | 525 |
| 185 | 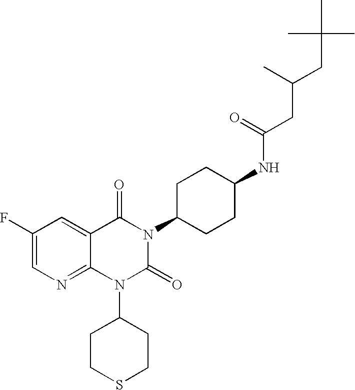 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3,5,5-trimethyl-hexanamide | 519 |
| 186 | 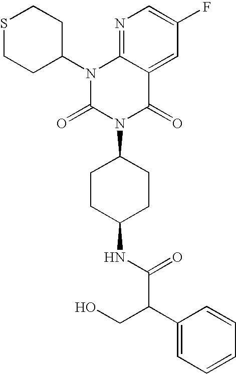 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-hydroxy-2-phenyl-propanamide | 527 |
| 187 | 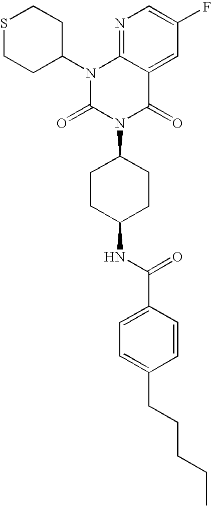 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-pentyl-benzamide | 553 |
| 188 | 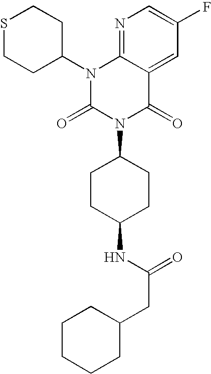 | 2-cyclohexyl-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 503 |
| 189 | 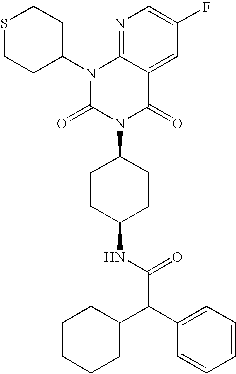 | 2-cyclohexyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-phenyl-acetamide | 579 |
| 190 | 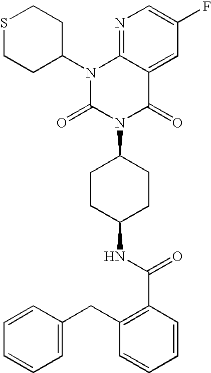 | 2-benzyl-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide | 573 |
| 191 | 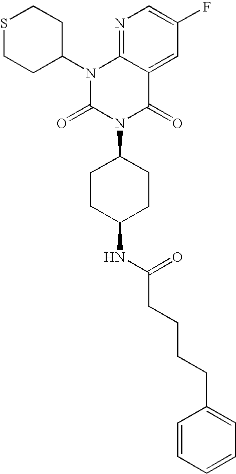 | N-[4-(9-fluoro-2,4-dioxo-5-tetrahydrothiopyran-4-yl-3,5,7-triazabicyclo[4.4.0]deca-6,8,10-trien-3-yl)cyclohexyl]-5-phenyl-pentanamide | 539 |
| 192 | 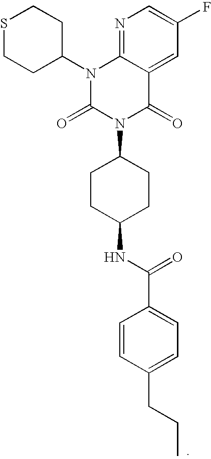 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-propyl-benzamide | 525 |
| 193 | 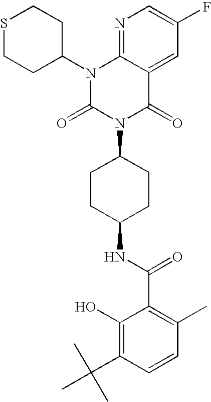 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-6-methyl-3-tert-butyl-benzamide | 569 |
| 194 | 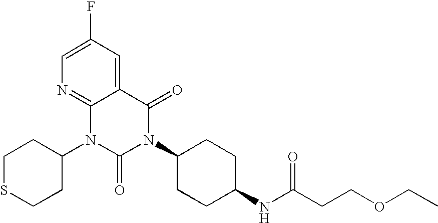 | 3-ethoxy-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide | 479 |
| 195 | 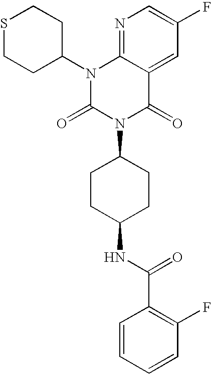 | 2-fluoro-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide | 501 |
| 196 | 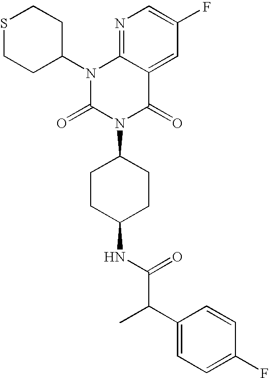 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-fluorophenyl)propanamide | 529 |
| 197 | 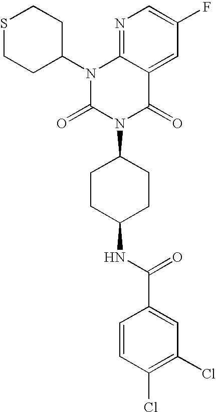 | 3,4-dichloro-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide | 551 |
| 198 | 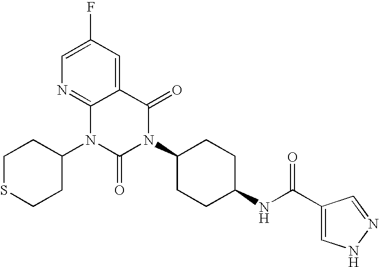 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1H-pyrazole-4-carboxamide | 473 |
| 199 | 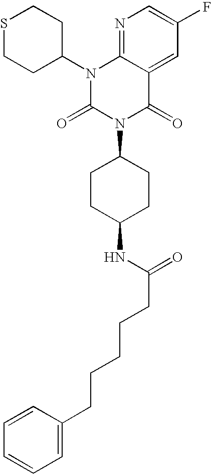 | N-[4-(9-fluoro-2,4-dioxo-5-tetrahydrothiopyran-4-yl-3,5,7-triazabicyclo[4.4.0]deca-6,8,10-trien-3-yl)cyclohexyl]-6-phenyl-hexanamide | 553 |
| 200 | 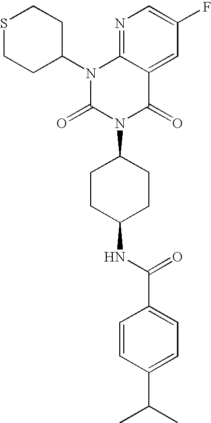 | N-[4-(9-fluoro-2,4-dioxo-5-tetrahydrothiopyran-4-yl-5,5,7-triazabicyclo[4.4.0]deca-6,8,10-trien-3-yl)cyclohexyl]-4-isopropyl-benzamide | 525 |
| 201 | 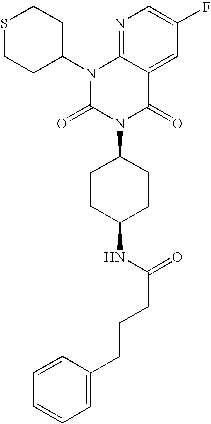 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-phenyl-butanamide. | 525 |
| 202 | 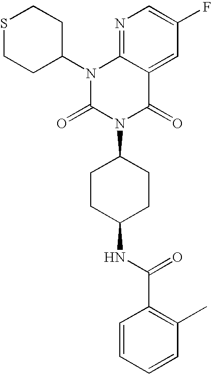 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-benzamide | 497 |
| 203 | 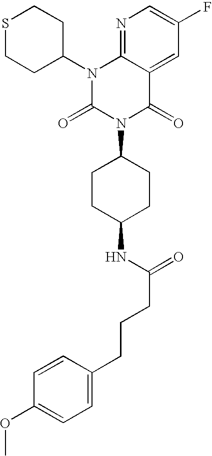 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(4-methoxyphenyl)butanamide | 555 |
| 204 | 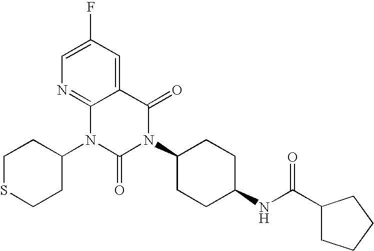 | N-[4-(9-fluoro-2,4-dioxo-5-tetrahydrothiopyran-4-yl-3,5,7-triazabicyclo[4.4.0]deca-6,8,10-trien-3-yl)cyclohexyl]cyclopentanecarboxamide | 475 |
| 205 | 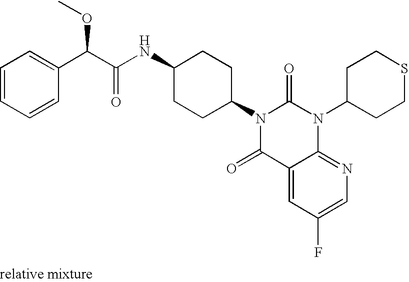 | (2R)-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methoxy-2-phenyl-acetamide | 527 |
| 206 | 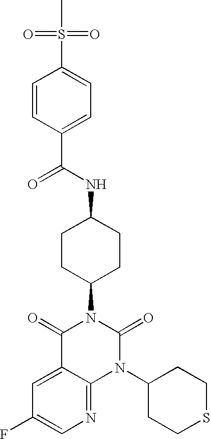 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-methylsulfonyl-benzamide | 561 |
| 207 | 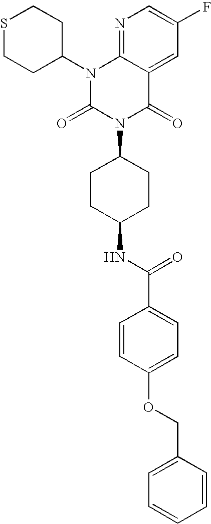 | 4-benzyloxy-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide | 589 |
| 208 | 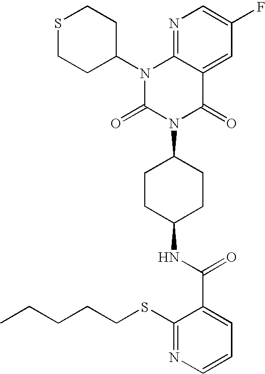 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-pentylsulfanyl-pyridine-3-carboxamide | 586 |
| 209 | 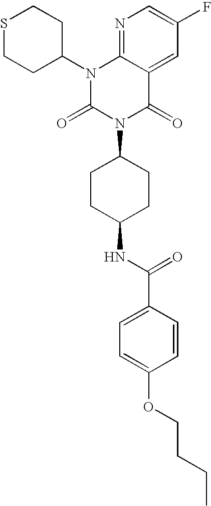 | 4-butoxy-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide | 555 |
| 210 | 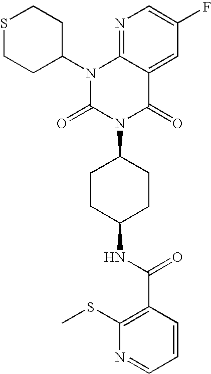 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methylsulfanyl-pyridine-3-carboxamide | 530 |
| 211 | 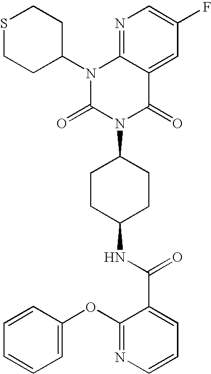 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-phenoxy-pyridine-3-carboxamide | 576 |
| 212 | 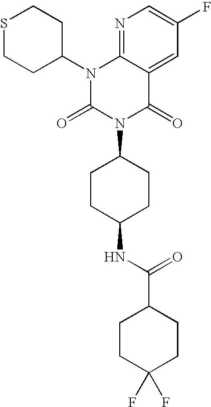 | 4,4-difluoro-N-[4-(9-fluoro-2,4-dioxo-5-tetrahydrothiopyran-4-yl-3,5,7-triazabicyclo[4.4.0]deca-6,8,10-trien-3-yl)cyclohexyl]cyclohexane-1-carboxamide | 525 |
| 213 | 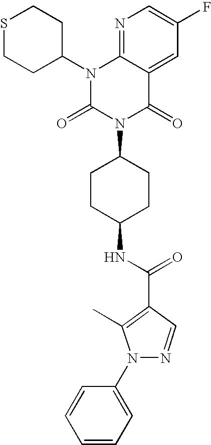 | N-[4-(9-fluoro-2,4-dioxo-5-tetrahydrothiopyran-4-yl-3,5,7-triazabicyclo[4.4.0]deca-6,8,10-trien-3-yl)cyclohexyl]-5-methyl-1-phenyl-pyrazole-4-carboxamide | 563 |
| 214 | 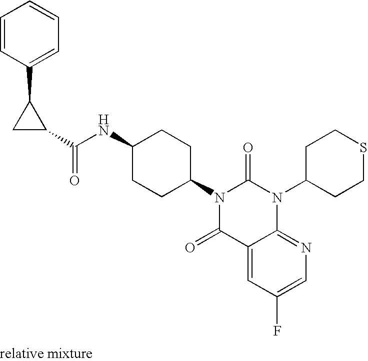 | (1R,2R)-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-phenyl-cyclopropane-1-carboxamide | 523 |
| 215 | 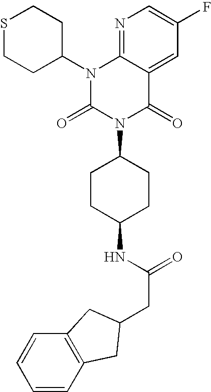 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-indan-2-yl-acetamide | 537 |
| 216 | 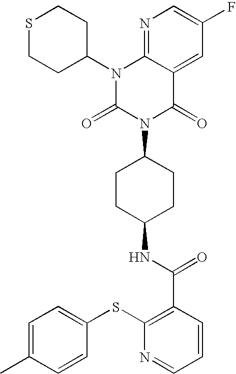 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(p-tolylsulfanyl)pyridine-3-carboxamide | 606 |
| 217 | 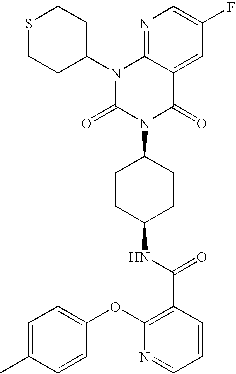 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-methylphenoxy)pyridine-3-carboxamide | 590 |
| 218 | 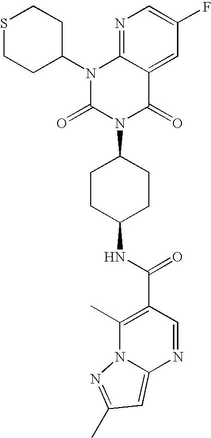 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2,8-dimethyl-1,5,9-triazabicyclo[4.3.0]nona-2,4,6,8-tetraene-3-carboxamide | 552 |
| 219 | 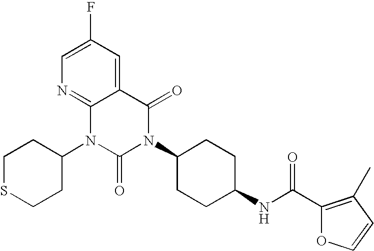 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methyl-furan-2-carboxamide | 487 |
| 220 | 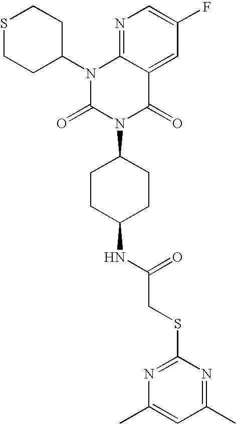 | 2-(4,6-dimethylpyrimidin-2-yl)sulfanyl-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 559 |
| 221 | 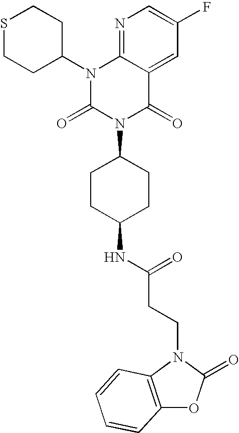 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(2-oxobenzooxazol-3-yl)propanamide | 568 |
| 222 | 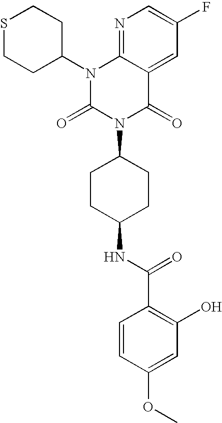 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-4-methoxy-benzamide | 529 |
| 223 | 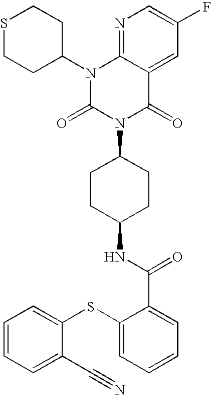 | 2-(2-cyanophenyl)sulfanyl-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide | 616 |
| 224 | 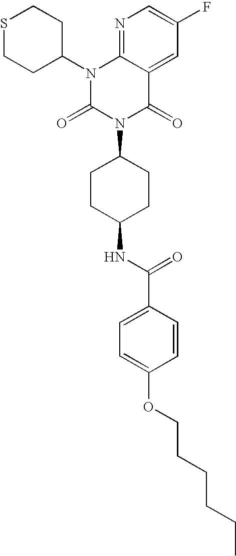 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-hexoxy-benzamide | 583 |
| 225 | 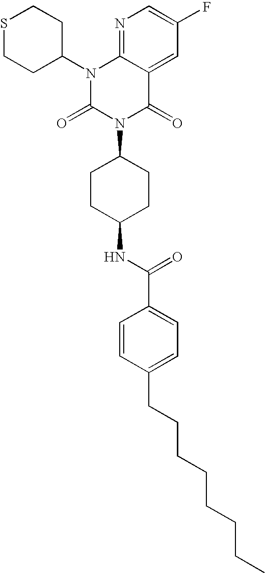 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-octyl-benzamide | 595 |
| 226 | 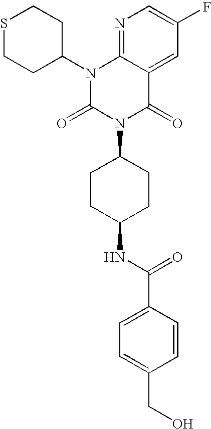 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(hydroxymethyl)benzamide | 513 |
| 227 | 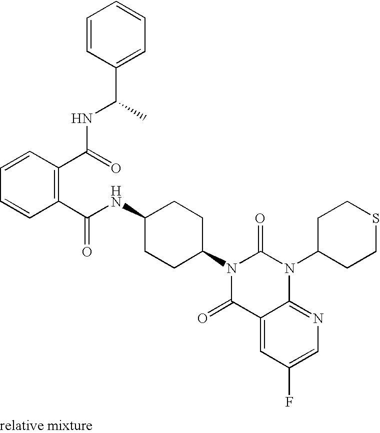 | N-[4-(9-fluoro-2,4-dioxo-5-tetrahydrothiopyran-4-yl-3,5,7-triazabicyclo[4.4.0]deca-6,8,10-trien-3-yl)cyclohexyl]-N-[(1S)-1-phenylethyl]phthalamide | 630 |
| 228 | 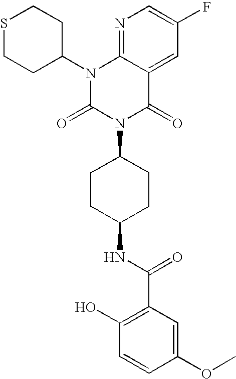 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-5-methoxy-benzamide | 529 |
| 229 | 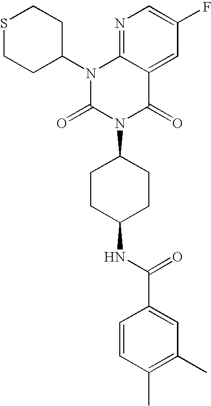 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3,4-dimethyl-benzamide | 511 |
| 230 | 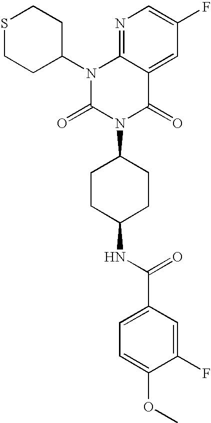 | 3-fluoro-N-[4-(9-fluoro-2,4-dioxo-5-tetrahydrothiopyran-4-yl-3,5,7-triazabicyclo[4.4.0]deca-6,8,10-trien-3-yl)cyclohexyl]-4-methoxy-benzamide | 531 |
| 231 | 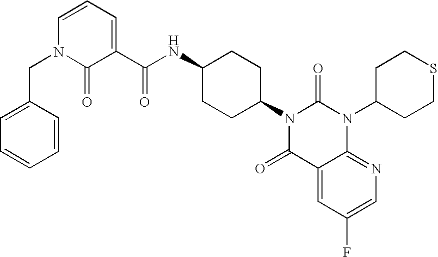 | 1-benzyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-oxo-pyridine-3-carboxamide | 590 |
| 232 | 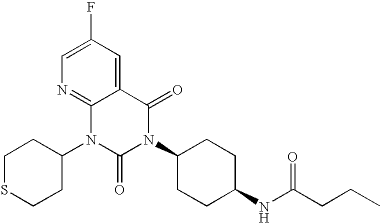 | N-[4-(9-fluoro-2,4-dioxo-5-tetrahydrothiopyran-4-yl-3,5,7-triazabicyclo[4.4.0]deca-6,8,10-trien-3-yl)cyclohexyl]butanamide | 449 |
| 233 | 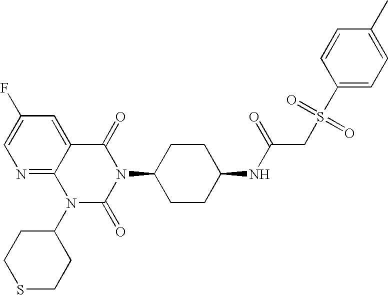 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(p-tolylsulfonyl)acetamide | 575 |
| 234 | 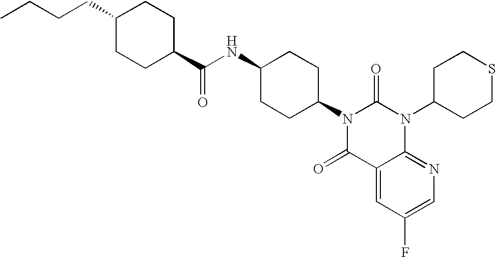 | 4-butyl-N-[4-(9-fluoro-2,4-dioxo-5-tetrahydrothiopyran-4-yl-3,5,7-triazabicyclo[4.4.0]deca-6,8,10-trien-3-yl)cyclohexyl]cyclohexane-1-carboxamide | 545 |
| 235 | 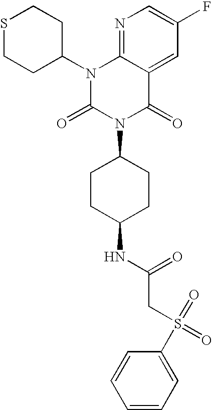 | 2-(benzenesulfonyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 561 |
| 236 | 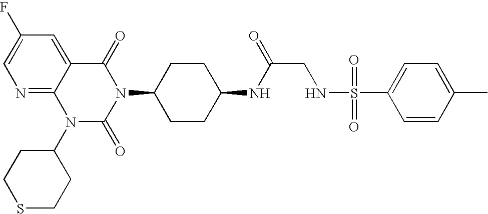 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(p-tolylsulfonylamino)acetamide | 590 |
| 237 | 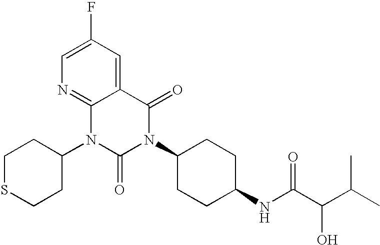 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-3-methyl-butanamide | 479 |
| 238 | 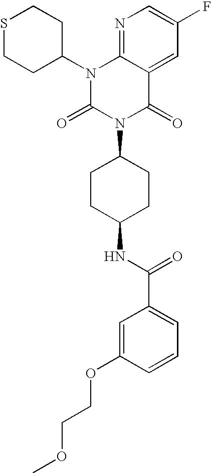 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(2-methoxyethoxy)benzamide | 557 |
| 239 | 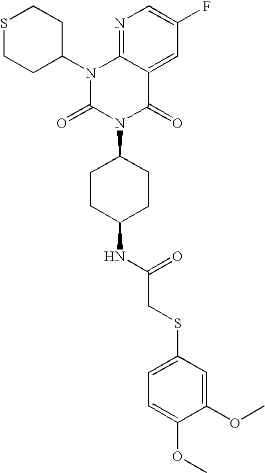 | 2-(3,4-dimethoxyphenyl)sulfanyl-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 589 |
| 240 | 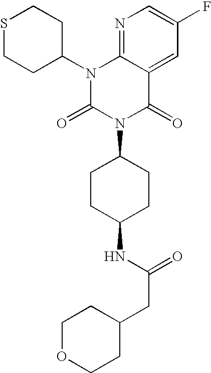 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-tetrahydropyran-4-yl-acetamide | 505 |
| 241 | 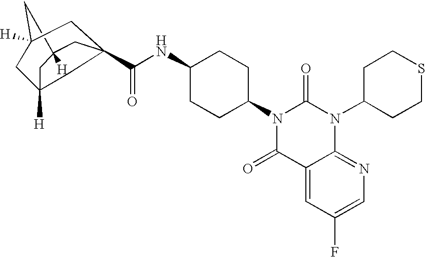 | N-[4-(9-fluoro-2,4-dioxo-5-tetrahydrothiopyran-4-yl-3,5,7-triazabicyclo[4.4.0]deca-6,8,10-trien-3-yl)cyclohexyl]carboxamide | 527 |
| 242 | 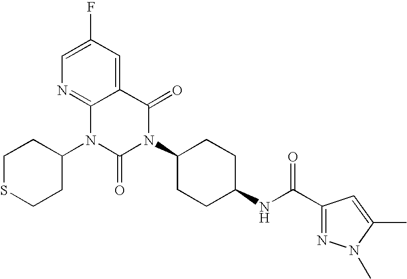 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1,5-dimethyl-pyrazole-3-carboxamide | 501 |
| 243 | 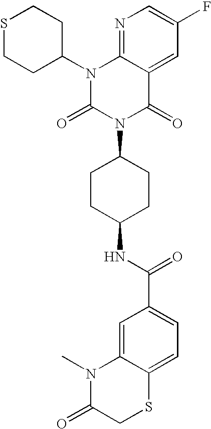 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-3-oxo-5-thia-2-azabicyclo[4.4.0]deca-6,8,10-triene-9-carboxamide | 584 |
| 244 | 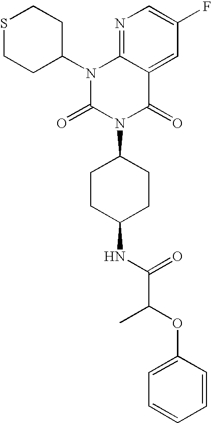 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-phenoxy-propanamide | 527 |
| 245 | 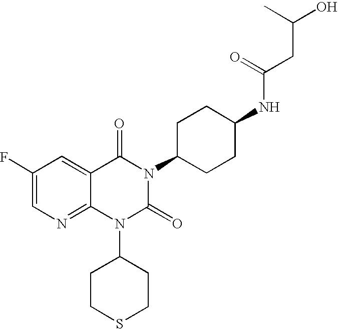 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-hydroxy-butanamide | 465 |
| 246 | 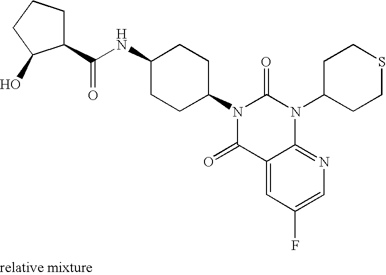 | (1R,2S)-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-cyclopantane-1-carboxamide | 491 |
| 247 | 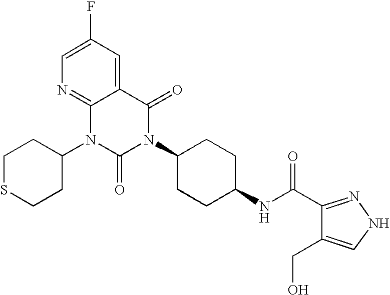 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(hydroxymethyl)-1H-pyrazole-3-carboxamide | 503 |
| 248 | 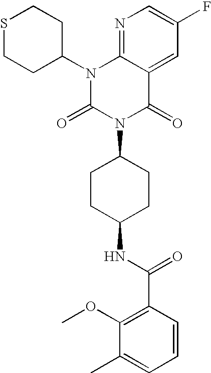 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methoxy-3-methyl-benzamide | 527 |
| 249 | 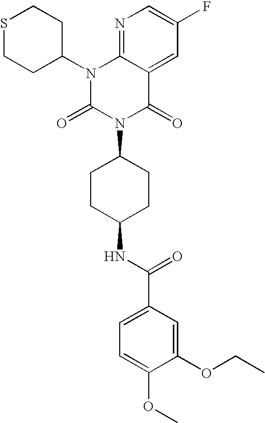 | 3-ethoxy-N-[4-(9-fluoro-2,4-dioxo-5-tetrahydrothiopyran-4-yl-3,5,7-triazabicyclo[4.4.0]deca-6,8,10-trien-3-yl)cyclohexyl]-4-methoxy-benzamide | 557 |
| 250 | 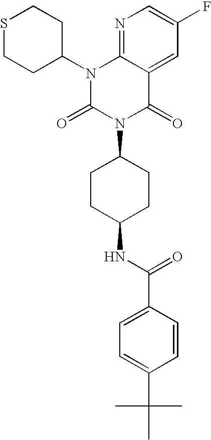 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-tert-butyl-benzamide | 539 |
| 251 | 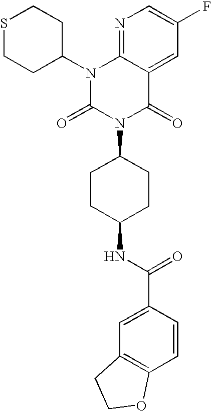 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2,3-dihydrobenzofuran-5-carboxamide | 525 |
| 252 | 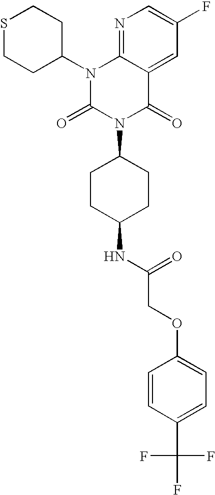 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-[4-(trifluoromethyl)phenoxy]acetamide | 581 |
| 253 | 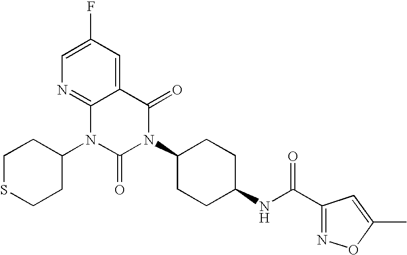 | N-[4-(9-fluoro-2,4-dioxo-5-tetrahydrothiopyran-4-yl-3,5,7-triazabicyclo[4.4.0]deca-6,8,10-trien-3-yl)cyclohexyl]-5-methyl-isoxazole-3-carboxamide | 488 |
| 254 | 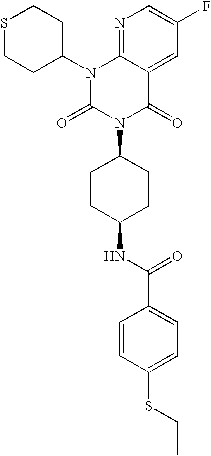 | 4-ethylsulfanyl-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide | 543 |
| 256 | 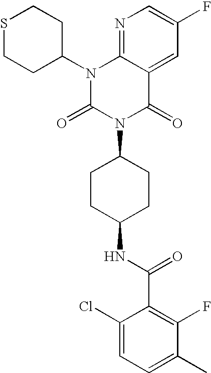 | 6-chloro-2-fluoro-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methyl-benzamide | 549 |
| 257 | 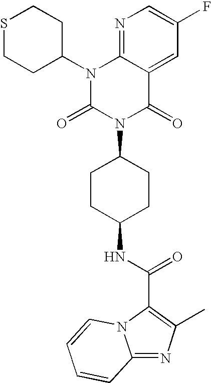 | N-[4-(9-fluoro-2,4-dioxo-5-tetrahydrothiopyran-4-yl-3,5,7-triazabicyclo[4.4.0]deca-6,8,10-trien-3-yl)cyclohexyl]-8-methyl-1,7-diazabicyclo[4.3.0]nona-2,4,6,8-tetraene-9-carboxamide | 537 |
| 258 | 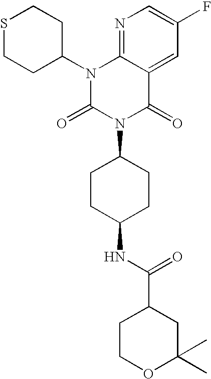 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2,2-dimethyl-tetrahydropyran-4-carboxamide | 519 |
| 259 | 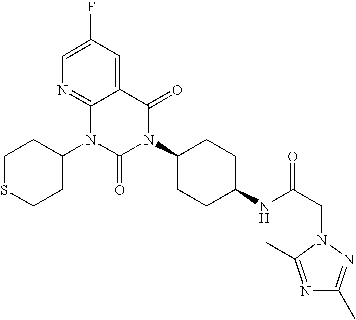 | 2-(3,5-dimethyl-1,2,4-triazol-1-yl)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 516 |
| 260 | 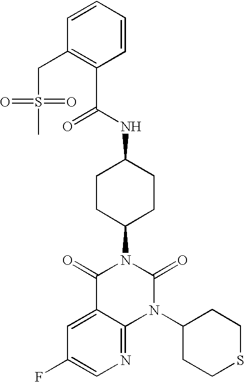 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(methylsulfonylmethyl) benzamide | 575 |
| 261 | 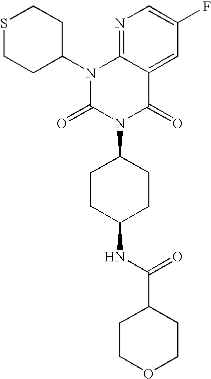 | N-[4-(9-fluoro-2,4-dioxo-5-tetrahydrothiopyran-4-yl-3,5,7-triazabicyclo[4.4.0]deca-6,8,10-trien-3-yl)cyclohexyl]tetrahydropyran-4-carboxamide | 491 |
| 262 | 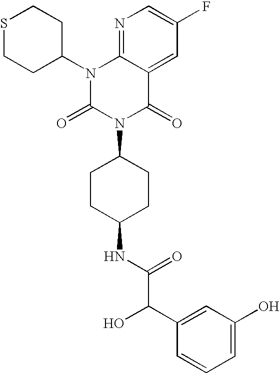 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-2-(3-hydroxyphenyl)acetamide | 529 |
| 263 | 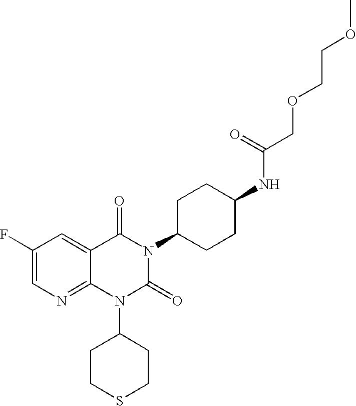 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(2-methoxyethoxy)acetamide | 495 |
| 264 | 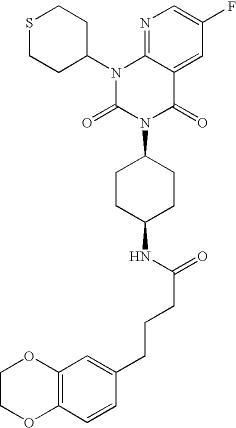 | 4-(2,5-dioxabicyclo[4.4.0]deca-7,9,11-trien-9-yl)-N-[4-(9-fluoro-2,4-dioxo-5-tetrahydrothiopyran-4-yl-3,5,7-triazabicyclo[4.4.0]deca-6,8,10-trien-3-yl)cyclohexyl]butanamide | 583 |
| 265 | 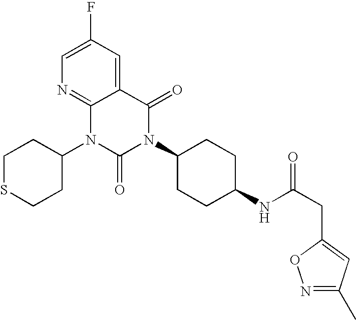 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(3-methylisoxazol-5-yl)acetamide | 502 |
| 266 | 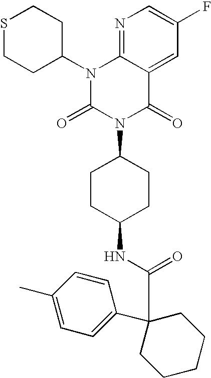 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-(p-tolyl)cyclohexane-1-carboxamide | 581 |
| 267 | 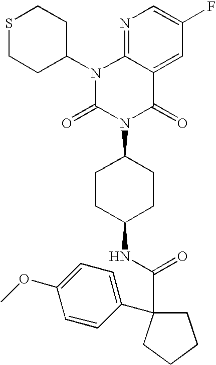 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-(4-methoxyphenyl)cyclopentane-1-carboxamide | 581 |
| 268 | 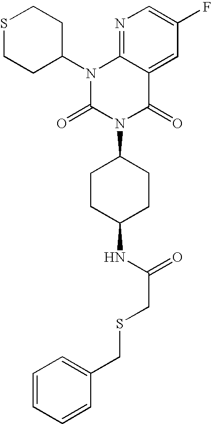 | 2-benzylsulfanyl-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 543 |
| 269 | 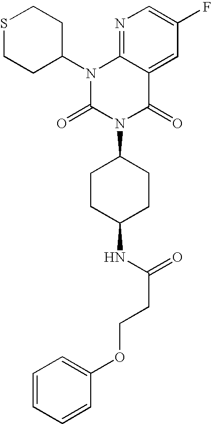 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-phenoxy-propanamide | 527 |
| 270 | 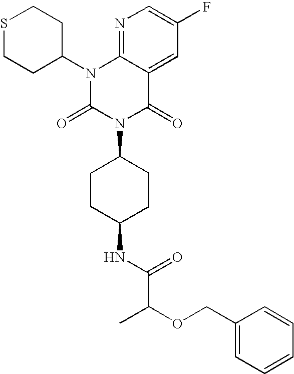 | 2-benzyloxy-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide | 541 |
| 271 | 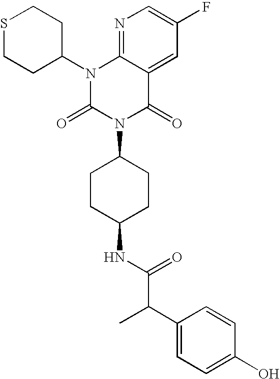 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-hydroxyphenyl)propanamide | 527 |
| 272 | 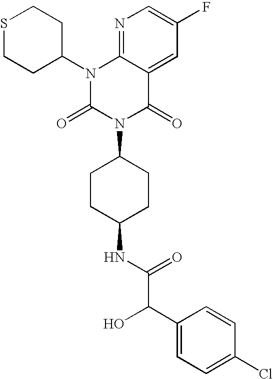 | 2-(4-chlorophenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-acetamide | 547 |
| 273 | 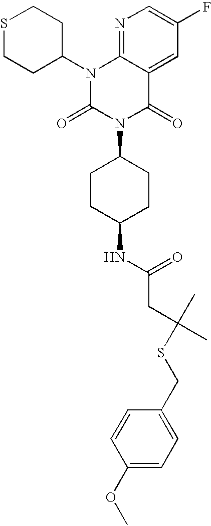 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-[(4-methoxyphenyl)methylsulfanyl]-3-methyl-butanamide | 615 |
| 274 | 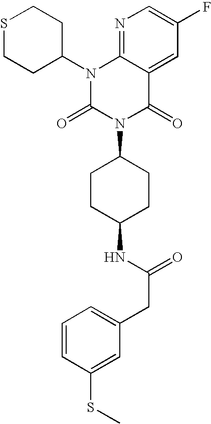 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(3-methylsulfanylphenyl) acetamide | 543 |
| 275 | 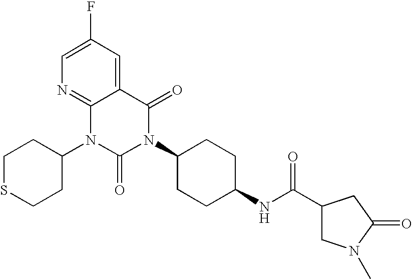 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-methyl-5-oxo-pyrrolidine-3-carboxamide | 504 |
| 276 | 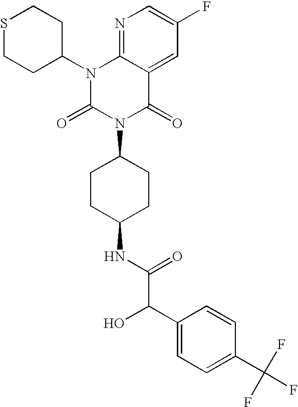 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-2-[4-(trifluoromethyl)phenyl]acetamide | 581 |
| 277 | 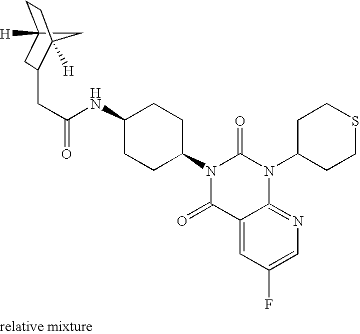 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-[(1S,4R)-norbornan-2-yl]acetamide | 515 |
| 278 | 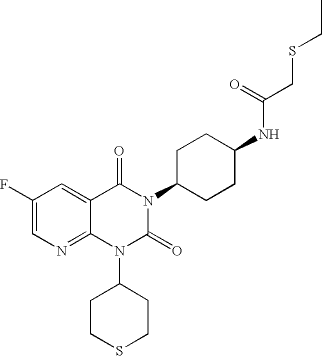 | 2-ethylsulfanyl-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 481 |
| 279 | 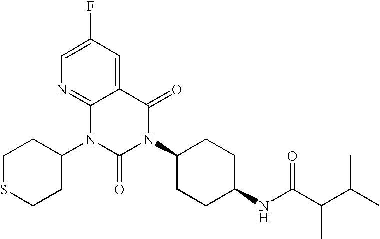 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2,3-dimethyl-butafanamide | 477 |
| 280 | 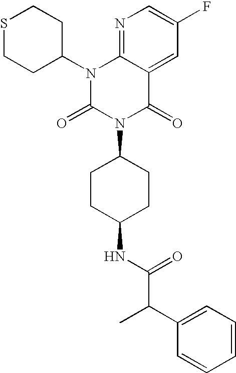 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-phenyl-propanamide | 511 |
| 281 | 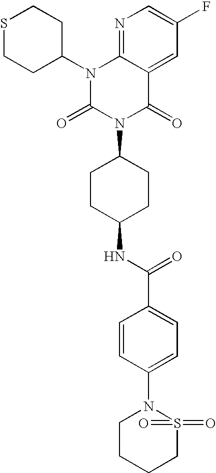 | 4-(1,1-dioxothiazinan-2-yl)-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide | 616 |
| 282 | 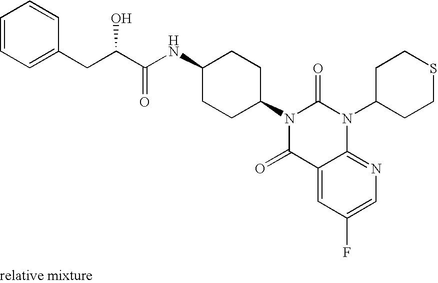 | (2S)-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-3-phenyl-propanamide | 527 |
| 283 | 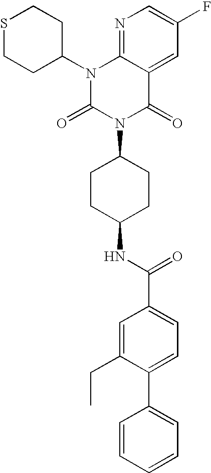 | 3-ethyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-4-phenyl-benzamide | 587 |
| 284 | 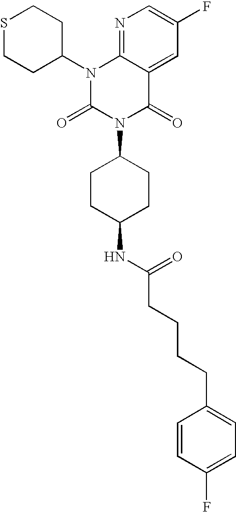 | N-[4-(9-fluoro-2,4-dioxo-5-tetrahydrothiopyran-4-yl-3,5,7-triazabicyclo[4.4.0]deca-6,8,10-trien-3-yl)cyclohexyl]-5-(4-fluorophenyl)pentanamide | 557 |
| 285 | 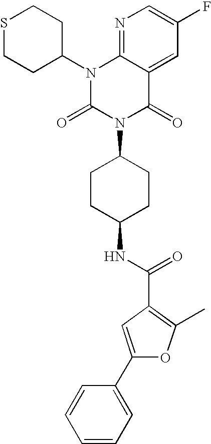 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-5-phenyl-furan-3-carboxamide | 563 |
| 286 | 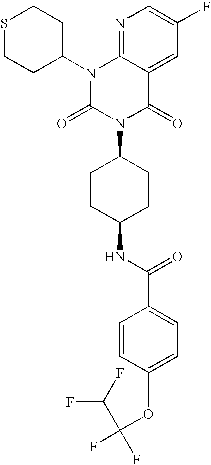 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(1,1,2,2-tetrafluoroethoxy)benzamide | 599 |
| 287 | 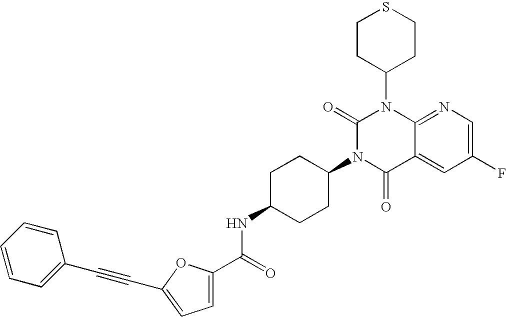 | N-[4-(9-fluoro-2,4-dioxo-5-tetrahydrothiopyran-4-yl-3,5,7-triazabicyclo[4.4.0]deca-6,8,10-trien-3-yl)cyclohexyl]-5-(2-phenylethynyl)furan-2-carboxamide | 573 |
| 288 | 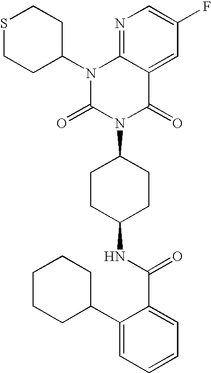 | 2-cyclohexyl-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide | 565 |
| 289 | 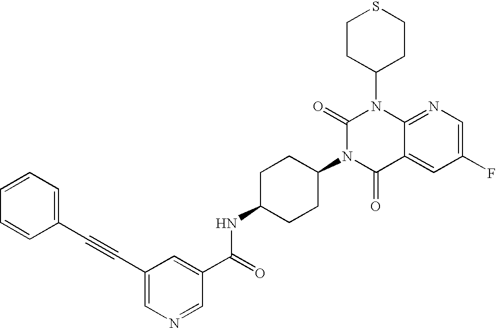 | N-[4-(9-fluoro-2,4-dioxo-5-tetrahydrothiopyran-4-yl-3,5,7-triazabicyclo[4.4.0]deca-6,8,10-trien-3-yl)cyclohexyl]-5-(2-phenylethynyl)pyridine-3-carboxamide | 584 |
| 290 | 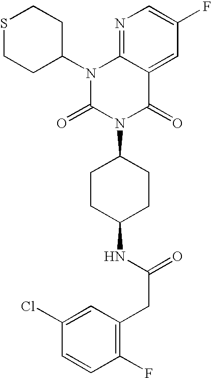 | 2-(5-chloro-2-fluoro-phenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 549 |
| 291 | 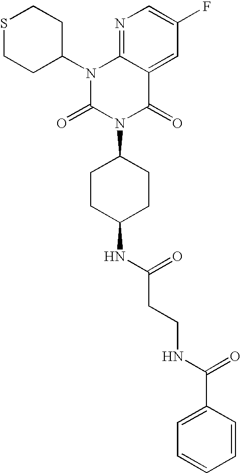 | N-[2-[[4-(9-fluoro-2,4-dioxo-5-tetrahydrothiopyran-4-yl-3,5,7-triazabicyclo[4.4.0]deca-6,8,10-trien-3-yl)cyclohexyl]carbamoyl]ethyl]benzamide | 554 |
| 293 | 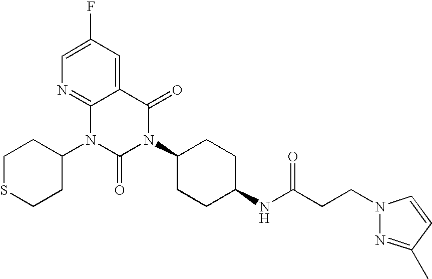 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(3-methylpyrazol-1-yl)propanamide | 515 |
| 295 | 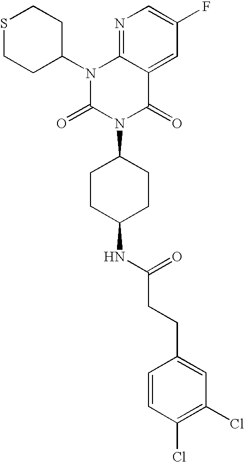 | 3-(3,4-dichlorophenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide | 579 |
| 296 | 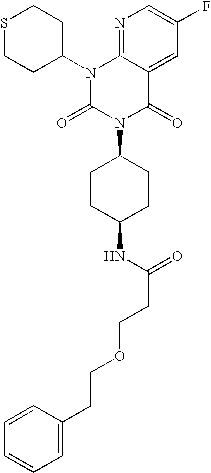 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-phenethyloxy-propanamide | 555 |
| 298 | 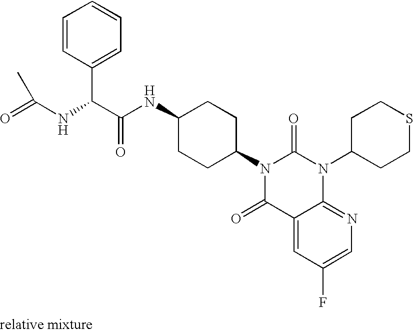 | (2R)-2-acetamido-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-phenyl-acetamide | 554 |
| 299 | 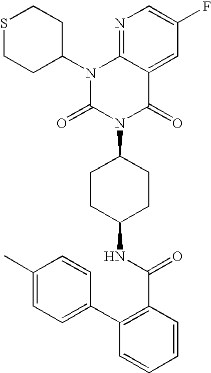 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(p-tolyl)benzamide | 573 |
| 300 | 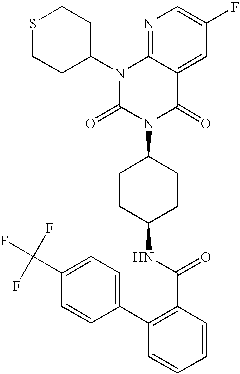 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-[4-(trifluoromethyl)phenyl]benzamide | 627 |
| 301 | 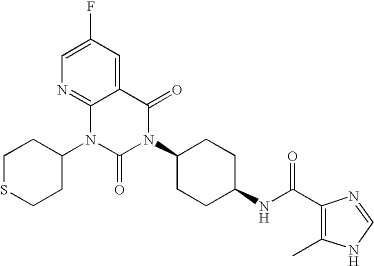 | N-[4-(9-fluoro-2,4-dioxo-5-tetrahydrothiopyran-4-yl-3,5,7-triazabicyclo[4.4.0]deca-6,8,10-trien-3-yl)cyclohexyl]-5-methyl-1H-imidazole-4-carboxamide | 487 |
| 302 | 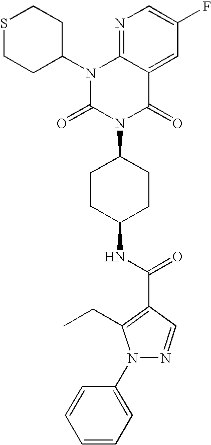 | 5-ethyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-1-phenyl-pyrazole-4-carboxamide | 577 |
| 303 | 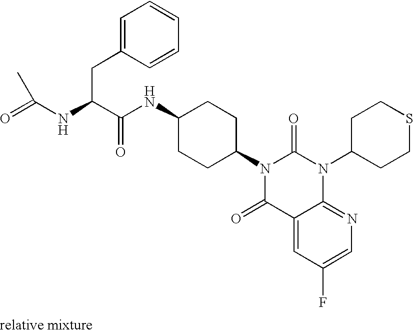 | (2S)-2-acetamido-N-[4-(9-fluoro-2,4-dioxo-5-tetrahydrothiopyran-4-yl-3,5,7-triazabicyclo[4.4.0]deca-6,8,10-trien-3-yl)cyclohexyl]-3-phenyl-propanamide | 568 |
| 304 | 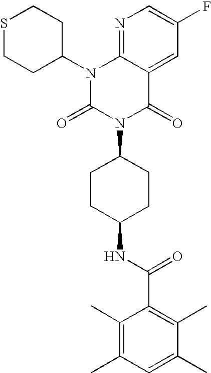 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2,3,5,6-tetramethyl-benzamide | 539 |
| 305 | 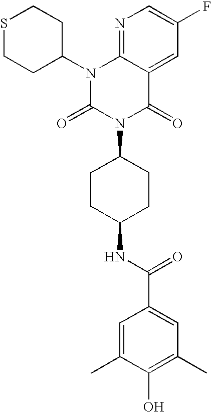 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-hydroxy-3,5-dimethyl-benzamide | 527 |
| 307 | 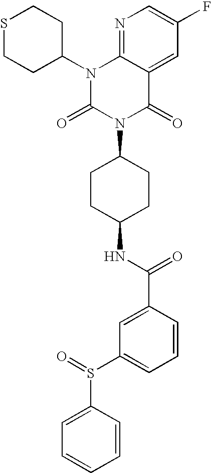 | 3-(benzenesulfinyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide | 607 |
| 309 | 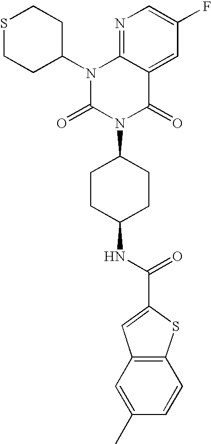 | N-[4-(9-fluoro-2,4-dioxo-5-tetrahydrothiopyran-4-yl-3,5,7-triazabicyclo[4.4.0]deca-6,8,10-trien-3-yl)cyclohexyl]-5-methyl-benzothiophene-2-carboxamide | 553 |
| 310 | 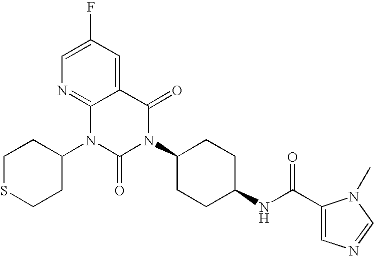 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methyl-imidazole-4-carboxamide | 487 |
| 311 | 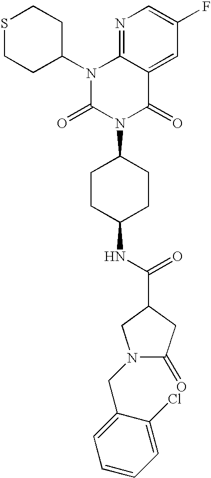 | 1-[(2-chlorophenyl)methyl]-N-[4-(9-fluoro-2,4-dioxo-5-tetrahydrothiopyran-4-yl-3,5,7-triazabicyclo[4.4.0]deca-6,8,10-trien-3-yl)cyclohexyl]-5-oxo-pyrrolidine-3-carboxamide | 614 |
| 312 | 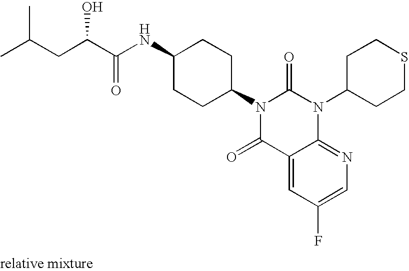 | (2S)-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-4-methyl-pentanamide | 493 |
| 313 | 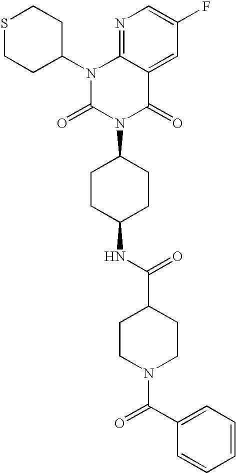 | 1-benzoyl-N-[4-(9-fluoro-2,4-dioxo-5-tetrahydrothiopyran-4-yl-3,5,7-triazabicyclo[4.4.0]deca-6,8,10-trien-3-yl)cyclohexyl]piperidine-4-carboxamide | 594 |
| 315 | 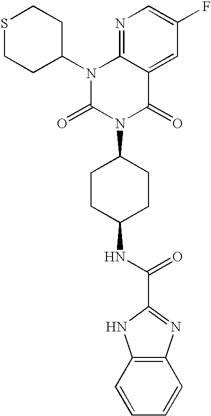 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1H-benzoimidazole-2-carboxamide | 523 |
| 316 | 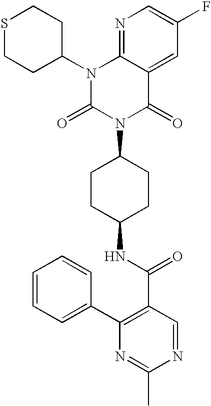 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-4-phenyl-pyrimidine-5-carboxamide | 575 |
| 317 | 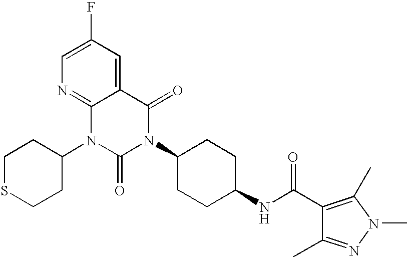 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1,3,5-trimethyl-pyrazole-4-carboxamide | 515 |
| 318 | 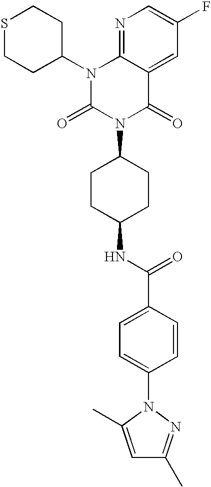 | 4-(3,5-dimethylpyrazol-1-yl)-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide | 577 |
| 319 | 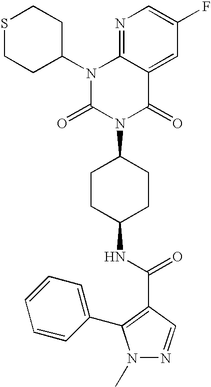 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-methyl-5-phenyl-pyrazole-4-carboxamide | 563 |
| 320 | 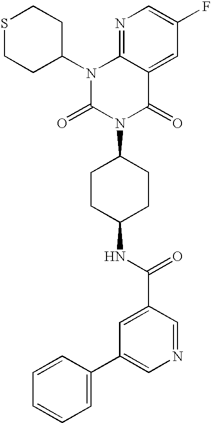 | N-[4-(9-fluoro-2,4-dioxo-5-tetrahydrothiopyran-4-yl-3,5,7-triazabicyclo[4.4.0]deca-6,8,10-trien-3-yl)cyclohexyl]-5-phenyl-pyridine-3-carboxamide | 560 |
| 321 | 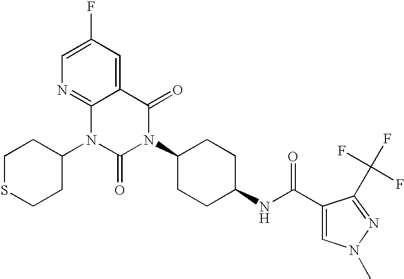 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-methyl-3-(trifluoromethyl)pyrazole-4-carboxamide | 555 |
| 322 | 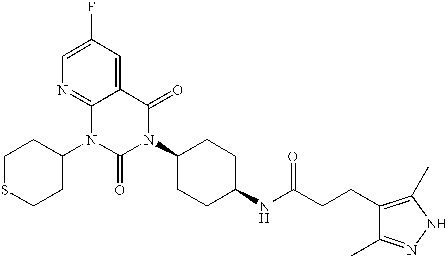 | 3-(3,5-dimethyl-1H-pyrazol-4-yl)-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide | 529 |
| 323 | 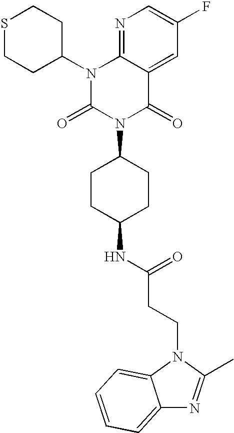 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(2-methylbenzoimidazol-1-yl)propanamide | 565 |
| 324 | 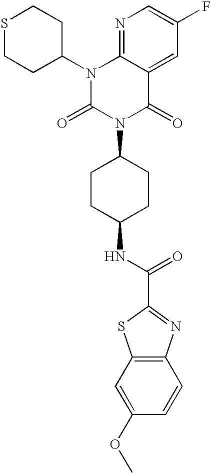 | N-[4-(9-fluoro-2,4-dioxo-5-tetrahydrothiopyran-4-yl-3,5,7-triazabicyclo[4.4.0]deca-6,8,10-trien-3-yl)cyclohexyl]-6-methoxy-benzothiazole-2-carboxamide | 570 |
| 326 | 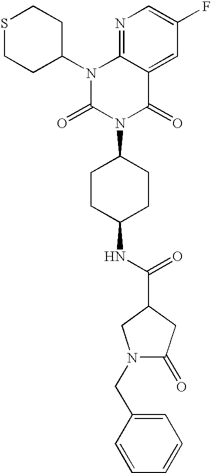 | 1-benzyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-5-oxo-pyrrolidine-3-carboxamide | 580 |
| 327 | 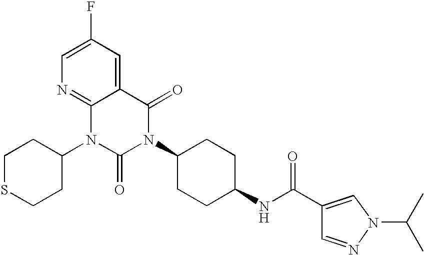 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-isopropyl-pyrazole-4-carboxamide | 515 |
| 328 | 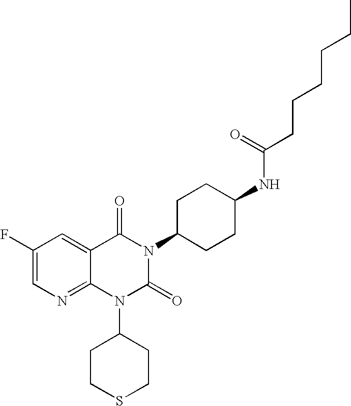 | N-[4-(9-fluoro-2,4-dioxo-5-tetrahydrothiopyran-4-yl-3,5,7-triazabicyclo[4.4.0]deca-6,8,10-trien-3-yl)cyclohexyl]heptanamide | 491 |
| 329 | 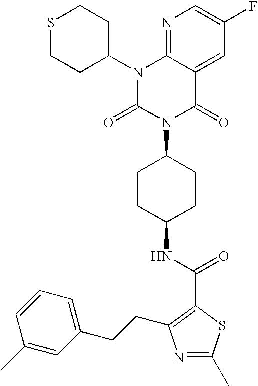 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-4-[2-(m-tolyl)ethyl]thiazole-5-carboxamide | 622 |
| 331 | 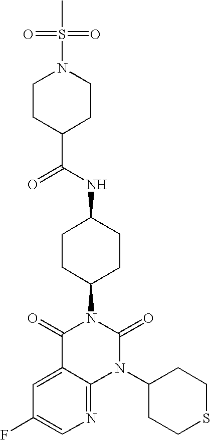 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-methylsulfonyl-piperidine-4-carboxamide | 568 |
| 332 | 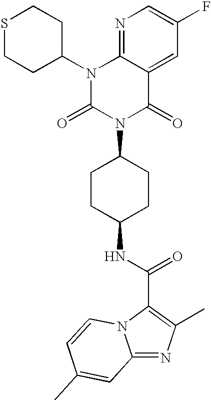 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4,8-dimethyl-1,7-diazabicyclo[4.3.0]nona-2,4,6,8-tetraene-9-carboxamide | 551 |
| 333 | 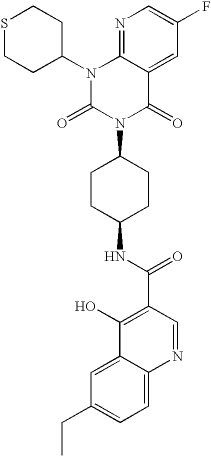 | 6-ethyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-4-hydroxy-quinoline-3-carboxamide | 578 |
| 334 | 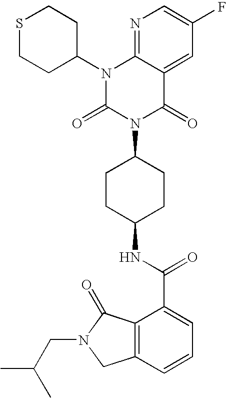 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-isobutyl-3-oxo-isoindoline-4-carboxamide | 594 |
| 335 | 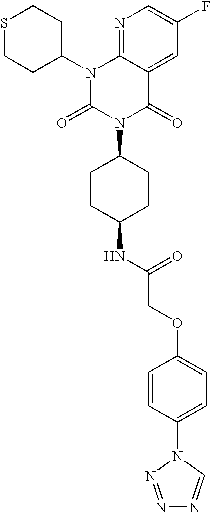 | N-[4-(9-fluoro-2,4-dioxo-5-tetrahydrothiopyran-4-yl-3,5,7-triazabicyclo[4.4.0]deca-6,8.10-trien-3-yl)cyclohexyl]-2-[4-(tetrazol-1-yl)phenozy]acetamide | 581 |
| 336 | 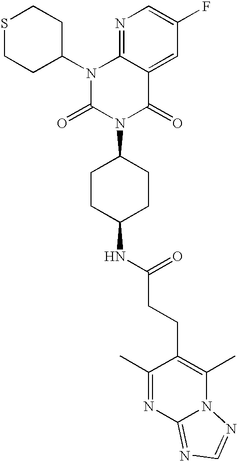 | 3-(2,4-dimethyl-1,5,7,9-tetrazabicyclo[4.3.0]nona-2,4,6,8-tetraen-3-yl)-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide | 581 |
| 337 | 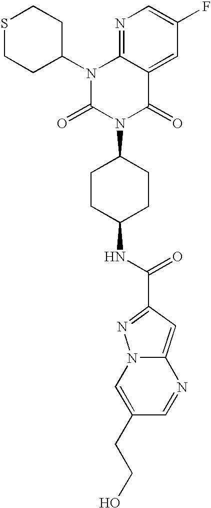 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(2-hydroxyethyl)-1,5,9-triazabicyclo[4.3.0]nona-2,4,6,8-tetraene-8-carboxamide | 568 |
| 338 | 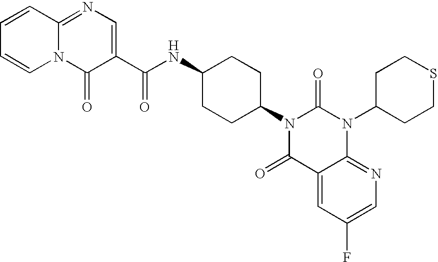 | N-[4-(9-fluoro-2,4-dioxo-5-tetrahydrothiopyran-4-yl-3,5,7-triazabicyclo[4.4.0]deca-6,8,10-trien-3-yl)cyclohexyl]-10-oxo-1,7-diazabicyclo[4.4.0]deca-2,4,6,8-tetraene-9-carboxamide | 551 |
| 339 | 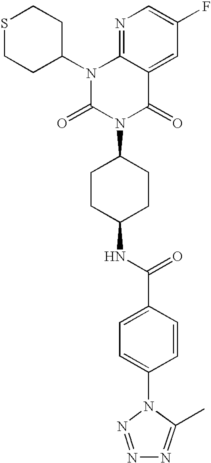 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(5-methyltetrazol-1-yl)benzamide | 565 |
| 342 | 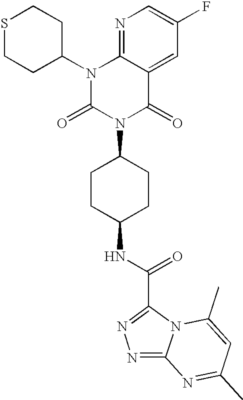 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2,4-dimethyl-1,5,7,8-tetrazabicyclo[4.3.0]nona-2,4,6,8-tetraene-9-carboxamide | 553 |
| 344 | 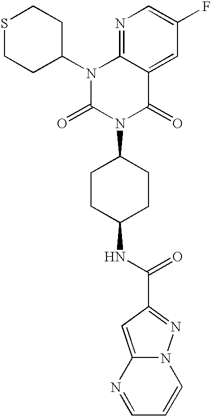 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1,5,9-triazabicyclo[4.3.0]nona-2,4,6,8-tetraene-8-carboxamide | 524 |
| 345 | 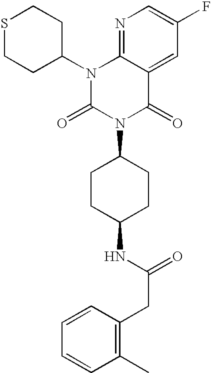 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(o-tolyl)acetamide | 511 |
| 346 | 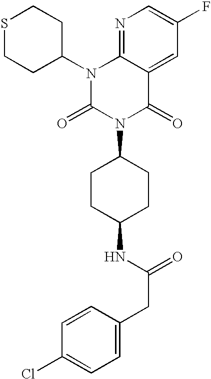 | 2-(4-chlorophenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 531 |
| 347 | 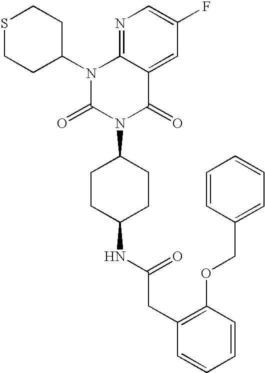 | 2-(2-benzyloxyphenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 603 |
| 348 | 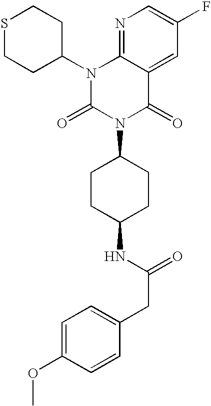 | N-[4-(9-fluoro-2,4-dioxo-5-tetrahydrothiopyran-4-yl-3,5,7-triazabicyclo[4.4.0]deca-6,8,10-trien-3-yl)cyolohexyl]-2-(4-methoxyphenyl)acetamide | 527 |
| 349 | 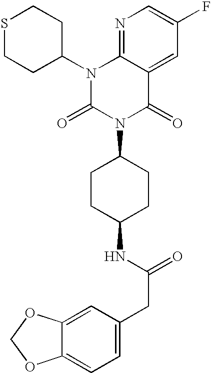 | 2-benzo[1,3]dioxol-5-yl-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 541 |
| 350 | 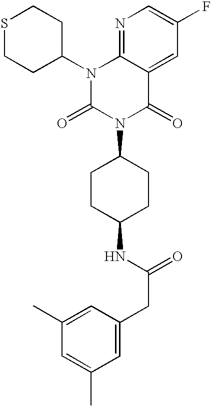 | 2-(3,5-dimethylphenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 525 |
| 351 | 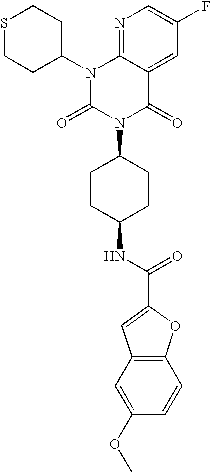 | N-[4-(9-fluoro-2,4-dioxo-5-tetrahydrothiopyran-4-yl-3,5,7-triazabicyclo[4.4.0]deca-6,8,10-trien-3-yl)cyclohexyl]-5-methoxy-benzofuran-2-carboxamide | 553 |
| 352 | 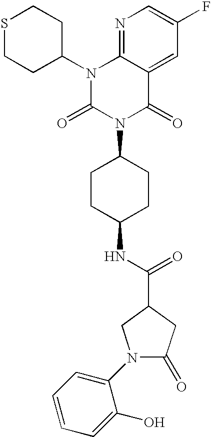 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-(2-hydroxyphenyl)-5-oxo-pyrrolidine-3-carboxamide | 582 |
| 353 | 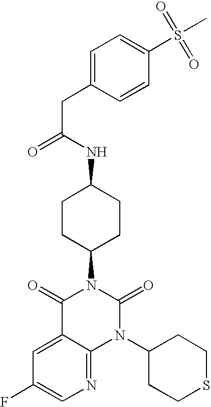 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-methylsulfonylphenyl)acetamide | 575 |
| 354 | 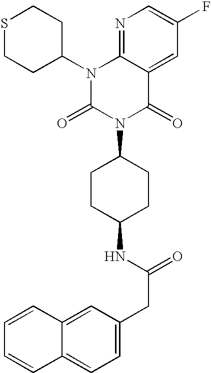 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(2-naphthyl)acetamide | 547 |
| 355 | 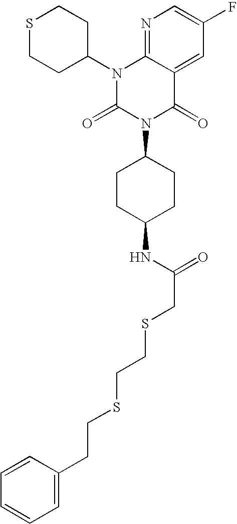 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(2-phenethylsulfanylethyl- sulfanyl)acetamide | 617 |
| 356 | 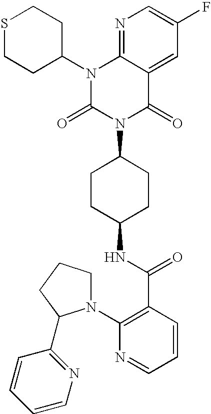 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-[2-(2-pyridyl)pyrrolidin-1-yl]pyridine-3-carboxamide | 630 |
| 357 | 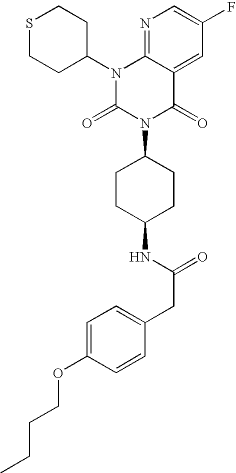 | 2-(4-butoxyphenyl)-N-[4-(9-fluoro-2,4-dioxo-5-tetrahydrothiopyran-4-yl-3,5,7-triazabicyclo[4.4.0]deca-6,8,10-trien-3-yl)cyclohexyl]acetamide | 569 |
| 359 | 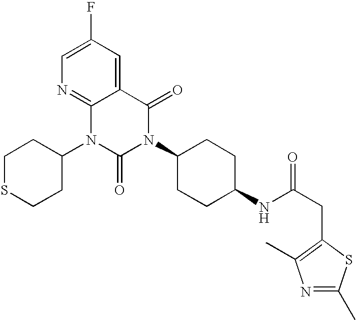 | 2-(2,4-dimethylthiazol-5-yl)-N-[4(-9-fluoro-2,4-dioxo-5-tetrahydrothiopyran-4-yl-3,5,7-triazabicyclo[4.4.0]deca-6,8,10-trien-3-yl)cyclohexyl]acetamide | 532 |
| 360 | 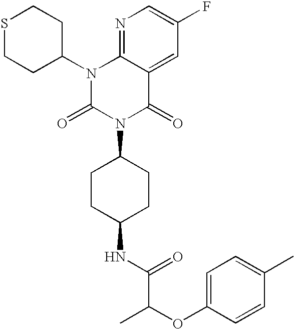 | N-[4-(9-fluoro-2,4-dioxo-5-tetrahydrothiopyran-4-yl-3,5,7-triazabicyclo[4.4.0]deca-5,8,10-trien-3-yl)cyclohexyl]-2-(4-methylphenoxy)propanamide | 541 |
| 361 | 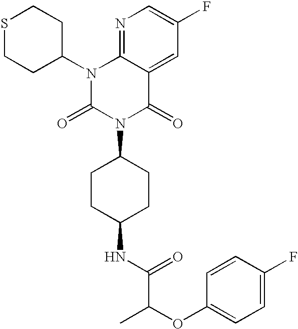 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-fluorophenoxy)propanamide | 545 |
| 363 | 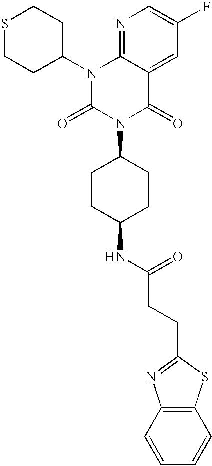 | 3-benzothiazol-2-yl-N-[4-(9-fluoro-2,4-dioxo-5-tetrahydrothiopyran-4-yl-3,5,7-triazabicyclo[4.4.0]deca-6,8,10-trien-3-yl)cyclohexyl]propanamide | 568 |
| 364 | 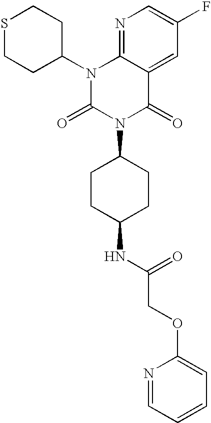 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(2-pyridyloxy)acetamide | 514 |
| 365 | 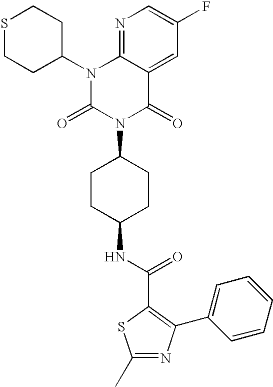 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-4-phenyl-thiazole-5-carboxamide | 580 |
| 366 | 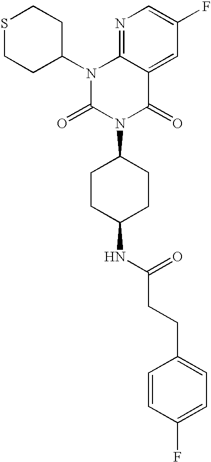 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-(4-fluorophenyl)propanamide | 529 |
| 367 | 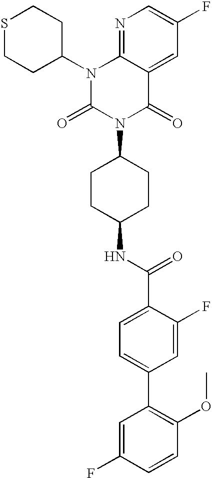 | 2-fluoro-N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(5-fluoro-2-methoxy-phenyl)benzamide | 625 |
| 368 | 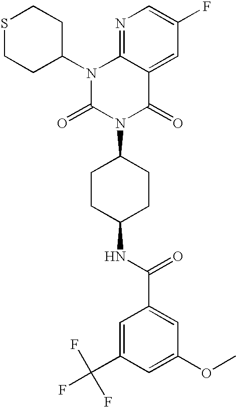 | N-[4-(9-fluoro-2,4-dioxo-5-tetrahydrothiopyran-4-yl-3,5,7-triazabicyclo[4.4.0]deca-6,8,10-trien-3-yl)cyclohexyl]-3-methoxy-5-(trifluoromethyl)benzamide | 581 |
| 369 | 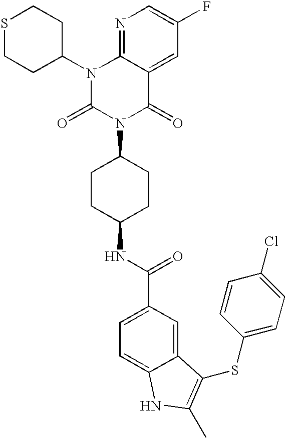 | 3-(4-chlorophenyl)sulfanyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-methyl-1H-indole-5-carboxamide | 678 |
| 370 | 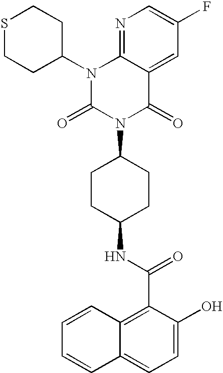 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-naphthalene-1-carboxamide | 549 |
| 371 | 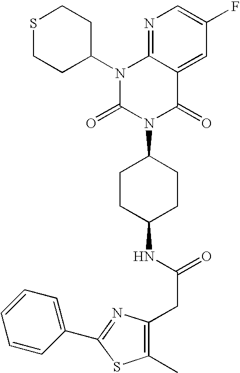 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(5-methyl-2-phenyl-thiazol-4-yl)acetamide | 594 |
| 372 | 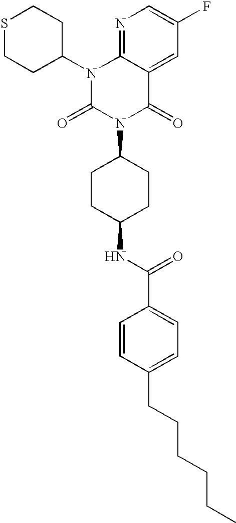 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-hexyl-benzamide | 567 |
| 373 | 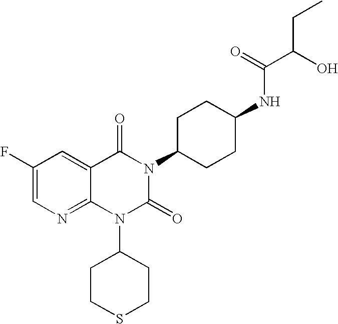 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-butanamide | 465 |
| 374 | 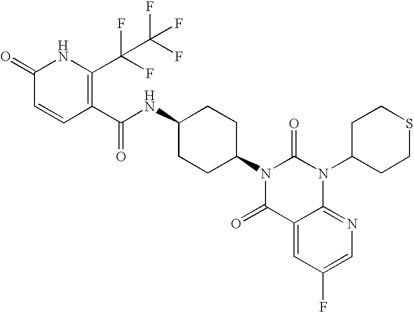 | N-[4-(9-fluoro-2,4-dioxo-5-tetrahydrothiopyran-4-yl-3,5,7-triazabicyclo[4.4.0]deca-6,8,10-trien-3-yl)cyclohexyl]-6-oxo-2-(1,1,2,2,2-pentafluoroethyl)-1H-pyridine-3-carboxamide | 618 |
| 375 | 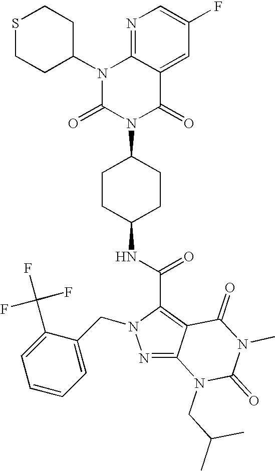 | N-[4-(9-fluoro-2,4-dioxo-5-tetrahydrothiopyran-4-yl-3,5,7-triazabicyclo[4.4.0]deca-6,8,10-trien-3-yl)cyclohexyl]-5-isobutyl-3-methyl-2,4-dioxo-8-[[2-(trifluoromethyl)phenyl]methyl]-3,5,7,8-tetrazabicyclo[4.3.0]nona-6,9-diene-9-carboxamide | 785 |
| 376 | 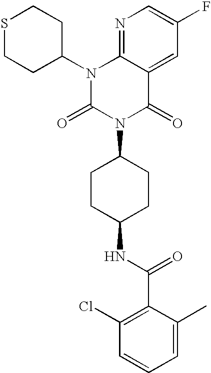 | 2-chloro-N-[4-(9-fluoro-2,4-dioxo-5-tetrahydrothiopyran-4-yl-3,5,7-triazabicyclo[4.4.0]deca-6,8,10-trien-3-yl)cyclohexyl]-6-methyl-benzamide | 531 |
| 377 | 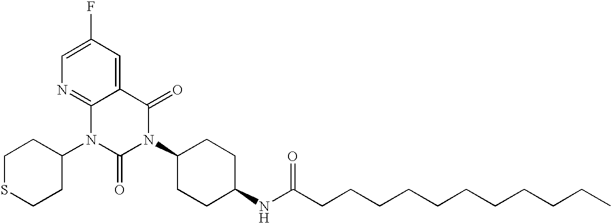 | N-[4-(9-fluoro-2,4-dioxo-5-tetrahydrothiopyran-4-yl-3,5,7-triazabicyclo[4.4.0]deca-6,8,10-trien-3-yl)cyclohexyl]dodecanamide | 561 |
| 378 | 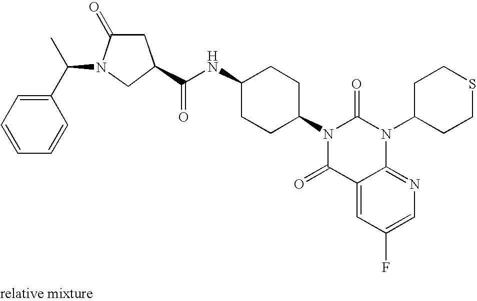 | (3R)-N-[4-(9-fluoro-2,4-dioxo-5-tetrahydrothiopyran-4-yl-3,5,7-triazabicyclo[4.4.0]deca-6,8,10-trien-3-yl)cyclohexyl]-5-oxo-1-[(1R)-1-phenylethyl]pyrrolidine-3-carboxamide | 594 |
| 380 | 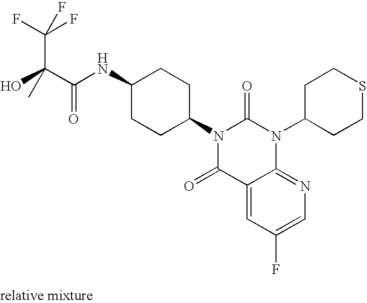 | (2R)-3,3,3-trifluoro-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-2-methyl-propanamide | 519 |
| 381 | 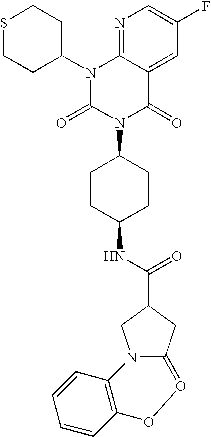 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-(2-methoxyphenyl)-5-oxo-pyrrolidine-3-carboxamide | 596 |
| 382 | 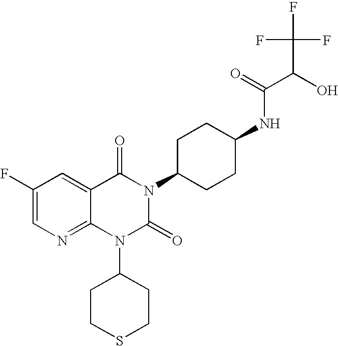 | 3,3,3-trifluoro-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-hydroxy-propanamide | 505 |
| 383 | 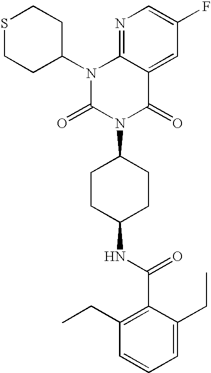 | 2,6-diethyl-N-[4-(9-fluoro-2,4-dioxo-5-tetrahydrothiopyran-4-yl-3,5,7-triazabicyclo[4.4.0]deca-6,8,10-trien-3-yl)cyclohexyl]benzamide | 539 |
| 384 | 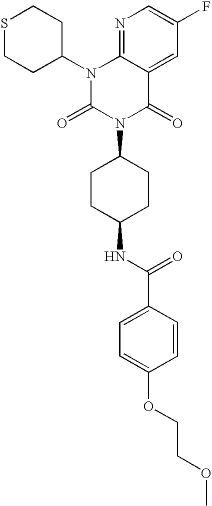 | N-[4-(9-fluoro-2,4-dioxo-5-tetrahydrothiopyran-4-yl-3,5,7-triazabicyclo[4.4.0]deca-6,8,10-trien-3-yl)cyclohexyl]-4-(2-methoxyethoxy)benzamide | 557 |
| 385 | 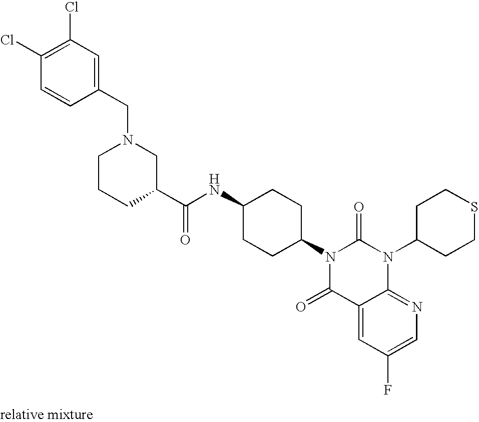 | (3R)-1-[(3,4-dichlorophenyl)methyl]-N-[4-(9-fluoro-2,4-dioxo-5-tetrahydrothiopyran-4-yl-3,5,7-triazabicyclo[4.4.0]deca-6,8,10-trien-3-yl)cyclohexyl]piperidine-3-carboxamide | 648 |
| 386 | 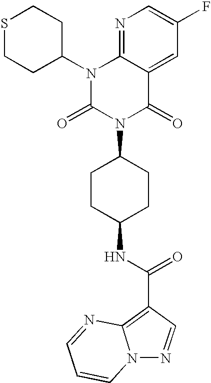 | N-[4-(9-fluoro-2,4-dioxo-5-tetrahydrothiopyran-4-yl-3,5,7-triazabicyclo[4.4.0]deca-6,8,10-trien-3-yl)cyclohexyl]-1-5,9-triazabicyclo[4.3.0]nona-2,4,6,8-tetraene-7-carboxamide | 524 |
| 387 | 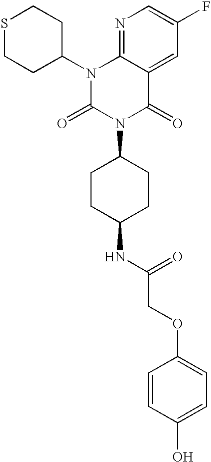 | N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-hydroxyphenoxy)acetamide | 529 |
| 388 | 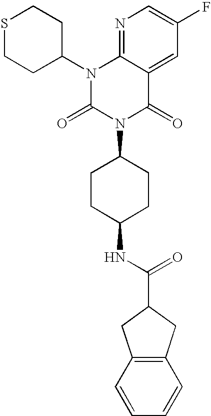 | N-[4-(9-fluoro-2,4-dioxo-5-tetrahydrothiopyran-4-yl-3,5,7-triazabicyclo[4.4.0]deca-6,8,10-trien-3-yl)cyclohexyl]indane-2-carboxamide | 523 |
| 389 | 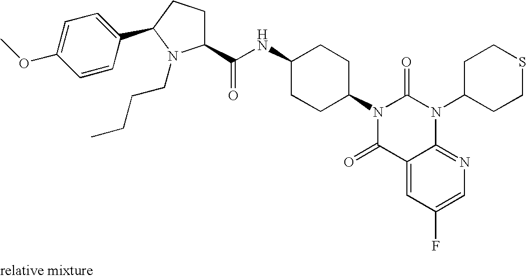 | (2S,5R)-1-butyl-N-[4-(9-fluoro-2,4-dioxo-5-tetrahydrothiopyran-4-yl-3,5,7-triazabicyclo[4.4.0]deca-6,8,10-trien-3-yl)cyclohexyl]-5-(4-methoxyphenyl)pyrrolidine-2-carboxamide | 638 |
| 390 | 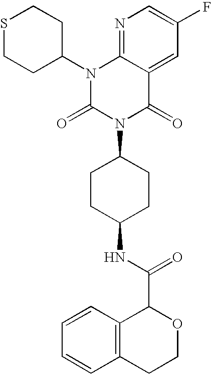 | N-[4-(9-fluoro-2,4-dioxo-5-tetrahydrothiopyran-4-yl-3,5,7-triazabicyclo[4.4.0]deca-6,8,10-trien-3-yl)cyclohexyl]isochroman-1-carboxamide | 539 |
| 391 | 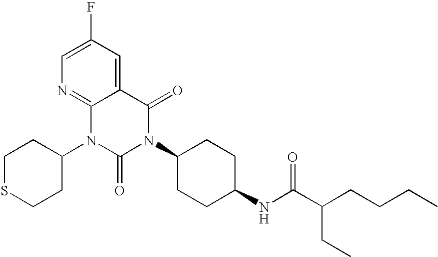 | 2-ethyl-N-[4-(9-fluoro-2,4-dioxo-5-tetrahydrothiopyran-4-yl-3,5,7-triazabicyclo[4.4.0]deca-6,8,10-trien-3-yl)cyclohexyl]hexanamide | 505 |
| 392 | 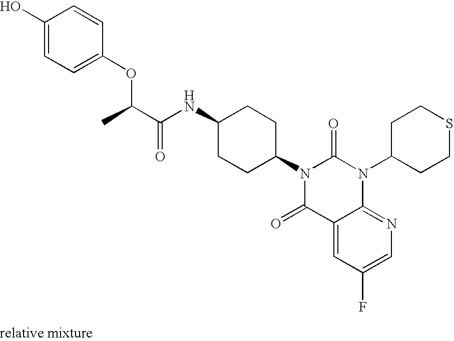 | (2R)-N-[4-(9-fluoro-2,4-dioxo-5-tetrahydrothiopyran-4-yl-3,5,7-triazabicyclo[4.4.0]deca-6,8,10-trien-3-yl)cyclohexyl]-2-(4-hydroxyphenoxy)propanamide | 543 |
| 393 | 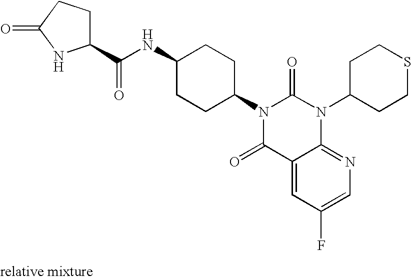 | (2S)-N-[4-(9-fluoro-2,4-dioxo-5-tetrahydrothiopyran-4-yl-3,5,7-triazabicyclo[4.4.0]deca-6,8,10-trien-3-yl)cyclohexyl]-5-oxo-pyrrolidine-2-carboxamide | 490 |
| 394 | 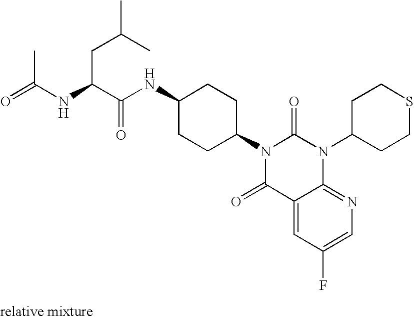 | (2S)-2-acetamido-N-[4-(9-fluoro-2,4-dioxo-5-tetrahydrothiopyran-4-yl-3,5,7-triazabicyclo[4.4.0]deca-6,8,10-trien-3-yl)cyclohexyl]-4-methyl-pentanamide | 534 |

### EXAMPLE 395

### Human Phosphodiesterase B2 Alpha Screen Assay

The assay uses recombinant Human Phosphodiesterase B2 (PDE4B2) produced in house (PrAZL0133), stored at −20° C. The substrate uses cAMP, part of the Alpha Screen cAMP kit (Perkin Elmer, Cat# 6760625M), stored at 4° C. The Alpha Screen kit also includes biotinylated cAMP, acceptor and donor beads.

The assay additions were as follows: Test compounds and controls were added to white 384-well flat-bottom plates (Greiner, Cat# 781075), 0.2 μl in 100% DMSO, followed by 10 μl PDE4B2 in reaction buffer. The reaction buffer constitution was: 50 mM Tris (pH 7.5), 8.3 mM MgCl2, 1.7 mM EGTA and 0.01% (w/v) Brij®35. The enzyme and the compounds were incubated at room temperature for 15 minutes. Then 10 μl cAMP in reaction buffer was added. The assay was stopped after 60 minutes incubation at room temperature by adding 10 μl acceptor beads in detection buffer with 40 mM EDTA. The detection buffer constitution was: 5 mM Tris (pH 7.5), 0.1% (w/v) BSA and 0.1% (v/v) Tween 20. This addition followed by an addition of 10 μl donor beads in detection buffer, with biotinylated cAMP. The plates were then incubated, dark at room temperature, for 5 hours followed by measurement on a Fusion™-α analyser. IC₅₀ values (presented in Table 4) were determined using Xlfit curve fitting using model 205.

TABLE 4

| Ex | PDE4B2 IC50 (nM) |
|---|---|
| 1 | 4.59 |
| 2 | 1.1 |
| 3 | 1.31 |
| 4 | 0.674 |
| 5 | 1.35 |
| 6 | 1.02 |
| 7 | 0.874 |
| 8 | 1.62 |
| 9 | 3.75 |
| 10 | 1.12 |
| 11 | 1.61 |
| 12 | 0.887 |
| 14 | 1.8 |
| 15 | 1.75 |
| 16 | 1.69 |
| 17 | 4.65 |
| 18 | 2.15 |
| 19 | 2.53 |
| 20 | 5.9 |
| 21 | 4.72 |
| 22 | 0.877 |
| 23 | 2.72 |
| 24 | 2.76 |
| 25 | 5.59 |
| 26 | 4.31 |
| 27 | 10.7 |
| 28 | 0.97 |
| 29 | 5.51 |
| 30 | 6.45 |
| 31 | 2.75 |

### EXAMPLE 396

### Human Phosphodiesterase B2 Radiometric Assay

The assay uses recombinant Human Phosphodiesterase B2 (PDE4B2) produced in house (PrAZL0163), stored at −20° C. This assay is based on the observation that 5′AMP, the product of the reaction catalysed by PDE4, binds preferentially to yttrium silicate SPA beads (Amersham Biosciences, UK) compared to the substrate, cAMP. Compounds at the appropriate concentration were preincubated at 30 C for 30 min with an assay buffer containing 50 mM HEPES (pH 7.5), 8.3 mM MgCl2, 1.7 mM EGTA, 0.01% (w/v) Brij®35 and 0.1 μg/mL recombinant PDE4B2. The reaction was started by the addition of [3H]cyclic AMP to give a final concentration of 8 nM, and was stopped 20 minutes after the addition of the substrate by the addition of yttrium silicate SPA beads containing 18 mM Zn SO4. Bound [3H]cyclic AMP was measured using a Topcount NXT (Packard Bioscience, UK). pIC₅₀ values (presented in Table 5) were determined using Xlfit3 curve fitting, using model 205.

TABLE 5

| Ex | PDE4B2 pIC₅₀ |
|---|---|
| 41 | 9.9 |
| 33 | 9.4 |
| 64b | 9.5 |
| 67 | 9.3 |

1. A compound of formula (I): 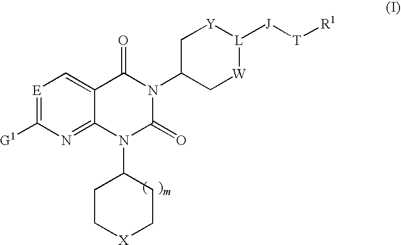
wherein:
X is S, S(O) or S(O)₂;
E is N or CE¹;
T is C(O) or S(O)₂;
W is (CH₂)ₙ;
Y is (CH₂)ₚ;
n and p are, independently 0 or 1;
m is 0 or 1;
L is CH or N;
when L is CH then J is NH; and when L is N then J is absent and T is bonded directly to L;
R¹ is C₁₋₁₀ alkyl (optionally substituted by halogen, hydroxyl, cyano, C₁₋₆ alkoxy, C₁₋₆ alkylthio, C₁₋₆ alkoxy(C₁₋₆ alkoxy), aryl, heteroaryl, aryloxy, heteroaryloxy, arylthio, heteroarylthio, aryl(C₁₋₄ alkoxy), aryl(C₁₋₄ alkylthio), C₃₋₇ cycloalkyl (optionally substituted by C₁₋₄ alkyl), CO₂H, CO₂(C₁₋₆ alkyl), NHC(O)R³, tetrahydropyranyl, C(O)NR²⁴R²⁵, S(O)₂R²⁶ or NHS(O)₂R²⁷), C₁₋₆ alkoxy, C₂₋₄ alkenyl (optionally substituted by phenyl), C₃₋₆ cycloalkyl (optionally substituted by hydroxyl, halogen, C₁₋₆ alkyl, C₁₋₆ alkoxy or phenyl), heterocyclyl (optionally substituted by oxo, hydroxy, C₁₋₆ alkyl, S(O)₂(C₁₋₄ alkyl), C(O)(C₁₋₄ alkyl), aryl, heteroaryl, aryl(C₁₋₄ alkyl), C(O)heteroaryl or heterocyclyl), aryl or heteroaryl;
R³ is C₁₋₆ alkyl or phenyl;
the foregoing phenyl, aryl and heteroaryl moieties of R¹ and R³ are, independently, optionally substituted by: halogen, cyano, nitro, hydroxy, S(O)_{q}R⁴, OC(O)NR⁵R⁶, NR⁷R⁸, NR⁹C(O)R¹⁰, NR¹¹C(O)NR¹²R¹³, S(O)₂NR¹⁴R¹⁵, NR¹⁶S(O)₂R¹⁷, C(O)NR¹⁸R¹⁹, C(O)R²⁰, CO₂R²¹, NR²²CO₂R²³, C₁₋₁₀ alkyl, C₁₋₆ hydroxyalkyl, C₁₋₆ haloalkyl, C₁₋₆ alkoxy(C₁₋₆)alkyl, C₁₋₆ alkyl(S(O)₂(C₁₋₄ alkyl)), di(C₁₋₆)alkylamino(C₁₋₆)alkyl, C₁₋₆ alkoxy, C₁₋₆ haloalkoxy, C₁₋₆ alkoxy(C₁₋₆)alkoxy, C₁₋₆ alkylthio, C₂₋₆ alkenyl, C₂₋₆ alkynyl (optionally substituted by phenyl), C₃₋₁₀ cycloalkyl (itself optionally substituted by C₁₋₄ alkyl or oxo), methylenedioxy, OCH₂CH₂O, difluoromethylenedioxy, heterocyclyl (optionally substituted by hydroxy, C₁₋₄ alkyl, phenyl or heteroaryl), phenyl, phenyl(C₁₋₄)alkyl, phenoxy, phenylthio, phenyl(C₁₋₄)alkoxy, heteroaryl, heteroaryl(C₁₋₄)alkyl, heteroaryloxy or heteroaryl(C₁₋₄)alkoxy; wherein any of the immediately foregoing phenyl and heteroaryl moieties are optionally substituted with halogen, hydroxy, nitro, S(O)ᵣ(C₁₋₄ alkyl), S(O)₂NH₂, S(O)₂NH(C₁₋₄ alkyl), S(O)₂N(C₁₋₄ alkyl)₂, cyano, C₁₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃;
E¹ and G¹ are, independently, hydrogen, halogen, cyano, hydroxy, C₁₄ alkyl, C₁₋₄ alkoxy, CF₃ or OCF₃;
q and r are, independently, 0, 1 or 2;
R⁴. R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶ and R²⁷ are, independently, C₁₋₁₀ alkyl {optionally substituted by halogen, hydroxy or C₁₋₆ alkoxy, or phenyl or heteroaryl both optionally substituted as recited below}, CH₂(C₂₋₆ alkenyl), phenyl {itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃} or heteroaryl {itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃};
R⁵, R⁶, R⁷, R⁸, R⁹, R¹⁰, R¹¹, R¹², R¹³, R¹⁴, R¹⁵, R¹⁶, R¹⁸, R¹⁹, R²⁰, R²¹, R²², R²³, R²⁴ and R²⁵ can also be hydrogen;
or a N-oxide thereof; or a pharmaceutically acceptable salt thereof.

2. A compound of formula (I) as claimed in claim 1 wherein E is CE¹.

3. A compound of formula (I) as claimed in claim 1 wherein E¹ is hydrogen or halogen.

4. A compound of formula (I) as claimed in claim 1, wherein n and p are both 1.

5. A compound of formula (I) as claimed in claim 1, wherein L is CH, J is NH and T is C(O).

6. A compound of formula (I) as claimed in claim 1 wherein G¹ is hydrogen.

7. A compound of formula (I) as claimed in claim 1 wherein m is 1.

8. A compound of formula (I) as claimed in claim 1 wherein X is S.

9. A compound of formula (I) as claimed in claim 1 wherein R¹ is C₁₋₆ alkyl (optionally substituted by halogen, hydroxyl, C₁₋₆ alkoxy, C₃₋₆ cycloalkyl (optionally substituted by hydroxyl, C₁₋₄ alkyl, C₁₋₄ alkoxy or phenyl), aryl or heteroaryl), C₃₋₇ cycloalkyl (optionally substituted by hydroxyl, halogen, C₁₋₆ alkyl, C₁₋₆ alkoxy or phenyl), heterocyclyl (optionally substituted by oxo, hydroxy, C₁₋₆ alkyl, S(O)₂(C₁₋₄ alkyl), C(O)(C₁₋₄ alkyl), aryl, heteroaryl, aryl(C₁₋₄ alkyl), C(O)heteroaryl or heterocyclyl), aryl or heteroaryl; the foregoing phenyl, aryl and heteroaryl moieties of R¹ are, independently, optionally substituted by: halogen, cyano, hydroxy, S(O)₂R⁴, NR⁷R⁸, NR¹¹C(O)NR¹²R¹³, S(O)₂NR¹⁴R¹⁵, C(O)NR¹⁸R¹⁹, C₁₋₁₀ alkyl, C₁₋₆ hydroxyalkyl, C₁₋₆ haloalkyl, C₁₋₆ alkoxy(C₁₋₆)alkyl, C₁₋₆ alkyl(S(O)₂(C₁₋₄ alkyl)), di(C₁₋₆)alkylamino(C₁₋₆)alkyl, C₁₋₆ alkoxy, C₁₋₆ haloalkoxy, C₁₋₆ alkoxy(C₁₋₆)alkoxy, C₃₋₁₀ cycloalkyl (itself optionally substituted by C₁₋₄ alkyl or oxo) or heterocyclyl (optionally substituted by hydroxy, C₁₋₄ alkyl, phenyl or heteroaryl); R⁴ is C₁₋₄ alkyl; and R⁷, R⁸, R¹¹, R¹², R¹³, R¹⁴, R¹⁵, R¹⁸ and R¹⁹ are, independently, hydrogen or C₁₋₄ alkyl.

10. A process for preparing a compound of formula (I) as claimed in claim 1, the process comprising removing the Boc protecting group from a compound of formula (II) 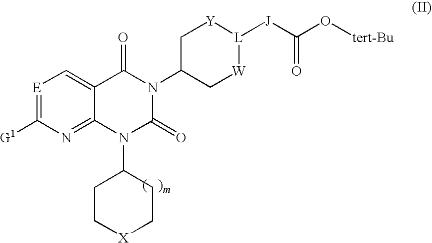
wherein m, G¹, E, Y, L, W, X and J are as defined in claim 1, and reacting the product so formed with a carboxylic acid, or derivative thereof, of formula (I11): 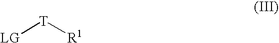
wherein R¹ and T are as defined in claim 1, and LG is a leaving group.

11. A pharmaceutical composition which comprises a compound of the formula (I), or a pharmaceutically acceptable salt thereof as claimed in claim 1, and a pharmaceutically acceptable adjuvant, diluent or carrier.

12. (canceled)

13. (canceled)

14. A method of treating a PDE 4 mediated disease state in a mammal suffering from, or at risk of, said disease, which comprises administering to a mammal in need of such treatment a therapeutically effective amount of a compound of formula (I), or a pharmaceutically acceptable salt thereof as claimed in claim 1.

15. A pharmaceutical product comprising, in combination, a first active ingredient which is a compound of formula (I), or a pharmaceutically acceptable salt thereof, as hereinbefore described, and at least one further active ingredient selected from:
a β2. adrenoceptor agonist,
a modulator of chemokine receptor function,
an inhibitor of kinase function,
a protease inhibitor,
a steroidal glucocorticoid receptor agonist,
an anticholinergic agent, and a
a non-steroidal glucocorticoid receptor agonist.

16. An intermediate compound of formula (II) 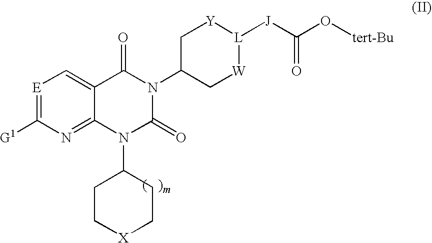
wherein m, G¹, E, Y, L, W, X and J are as defined in claim 1.
